(19) United States
(12) Patent Application Publication
(10) Pub. No.: US 2003/0060457 A1
(43) Pub. Date: Mar. 27, 2003

(54) Cellular proteins as targets for the treatment of pathogens resistant to drugs that target pathogen-encoded proteins

(76) Inventors: Priscilla A. Schaffer, Boston, MA (US); Luis M. Schang, Edmonton (CA)

Correspondence Address: MORGAN, LEWIS & BOCKIUS LLP, 1701 MARKET STREET, PHILADELPHIA, PA, 19103-2921, US

(21) Appl. No.: 09/905,695

(22) Filed: Dec. 6, 2000

Related U.S. Application Data
(63) Continuation-in-part of application No. 09/951,058, filed on Sep. 12, 2000.
(63) Continuation-in-part of application No. 09/656,592, filed on Sep. 7, 2000.
Application No. 09/656,592 is a continuation of application No. PCT/US99/16252, filed on Jul. 16, 1999.
(60) Provisional application No. 60/094,805, filed on Jul. 31, 1998.
(60) Provisional application No. 60/131,264, filed on Apr. 27, 1999.
(60) Provisional application No. 60/140,926, filed on Jun. 24, 1999.

(51) Int. Cl. A61K 31/553; A61K 31/52; A61K 31/4745; A61K 31/365; A61K 31/404; A61K 31/255

(52) U.S. Cl. 514/211.08; 514/263.4; 514/456; 514/473; 514/414; 514/285; 514/518

(57) ABSTRACT

The invention relates to the identification of cdk inhibitors as inhibitors of gene expression, replication and reactivation in pathogenic agents. Compositions and assays for the identification and use of such inhibitors are provided, as are methods of use of the inhibitors

### STATEMENT OF FEDERALLY SPONSORED RESEARCH AND DEVELOPMENT

The invention was made in part using funds obtained from the U.S. Government (National Institutes of Health Grant Nos. R01CA20260, P01NS35138) and the U.S. Government may have certain rights in the invention.

### BACKGROUND OF THE INVENTION

Conventional antiviral, antibiotic, antifungal and anti-parasitic drugs target proteins that are encoded by the respective pathogens, i.e., these types of drugs have been demonstrated to be specific for pathogen-encoded proteins. Frequently, variants of these pathogens arise that encode drug-resistant forms of the targeted proteins. Consequently, new drugs must be developed which incapacitate new drug-resistant forms of the targeted proteins. One method used in the art which diminishes the emergence of drug-resistant variants of pathogens is the use of combinations of drugs which target selected pathogen-encoded proteins. The combination approach can be a powerful therapeutic method, but this approach currently requires at least two distinct drugs that target two independent pathogen-encoded proteins. In the clinical setting, this type of combination therapy is being commonly used as a method of treatment against only one pathogen, the Human Immunodeficiency virus (HIV).

Herpesviruses are a family of large double stranded DNA-containing viruses many members of which are important animal and human pathogens. A common property of herpesviruses is their capacity to cause both acute (productive) and latent infections in the host, each of which is characterized by marked differences in patterns of viral transcription and DNA replication. Members of the herpesvirus family which are human pathogens include herpes simplex virus type 1(HSV-1), herpes simplex virus type 2 (HSV-2), varicella zoster virus (VZV), Epstein Barr virus (EBV), cytomegalovirus (CMV), human herpes virus type 6 (HHV-6), human herpes virus type 7 (HHV-7), and human herpesvirus type 8 (HHV-8), which is associated with Kaposi's sarcoma (Chang et al., 1995, Science 266:1865-1869). HSV-1 infection is associated with cold sores, keratoconjunctivitis, stromal keratitis, eczema herpeticum, gingivostomatitis, possibly atherosclerosis, meningoencephalitis and herpes encephalitis. HSV-2 infection is associated with genital herpes, systemic disease of newborns, keratitis, meningoencephalitis and herpes encephalitis. VZV infection is associated with chicken pox and shingles. EBV infection is associated with mononucleosis, Burkitt's lymphoma, nasopharyngeal carcinoma and assorted lymphomas. CMV infection is associated with CMV mononucleosis, congenital CMV and possibly atherosclerosis and restenosis. HHV-6 infection is associated with roseola infantum, possibly multiple sclerosis, and acute hepatitis in newborns and young children. It is not presently known what diseases HHV-7 is associated with and as stated above, HHV-8 is associated with Kaposi's sarcoma. Several of the human herpesviruses also cause disseminated infections in immunosuppressed patients, including patients having AIDS. Of all of the human herpesviruses, herpes simplex virus type 1 (HSV-1) has been most intensively studied.

Progression of cells through the eukaryotic cell cycle is regulated by a family of serine/threonine protein kinases called cyclin-dependent kinases or cdks (Draetta, 1994, Curr. Opin. Cell Biol. 6:842-846; Sherr, 1993, Cell 73:1059-1065; Sherr, 1994, Cell 79:551-555). Cdks comprises a family of at least nine enzymes, some of which have been studied in considerable detail. Cdk-1 complexes with both cyclins A and B in cells and plays a critical role in regulating the G2/M phase transition (Sherr, 1993, Cell 73:1059-1065). Cdk-3 has been implicated in progression of the cell into the S phase of the cell cycle (van den Heuvel E T al., 1993, Science 262:2050-2054). Cdk-5 functions in neural tissues to maintain the axon cytoskeleton (Lew et al., 1994, Nature 11:423-426). Cdk-5 is the only cdk whose function does not involve the cell cycle. Cdk-7 is the catalytic subunit of the cdk activating kinase, which plays a role in regulating cdk activity and it is also involved in transcription by phosphorylation of RNA polymerase II (Fesquet et al., 1993, EMBO J. 12:3111-3121; Fisher et al., 1994, Cell 78:713-724). It is generally believed that cdk-4 and cdk-6 regulate processes that are essential for progression of cells through mid to late G1 phase; whereas cdk-2 regulates processes that are involved in the initiation and progress of S phase (Draetta, 1994, Curr. Opin. Cell Biol. 6:842-846; Sherr, 1993, Cell 73:1059-1065; Sherr, 1994, Cell 79:551-555) Cdk-4 and cdk-6 are activated by association with one or another of the D-type (D1, D2, D3) cyclins (Bates et al., 1994, Oncogene 9:71-79; Matsushime et al., 1992, Cell 71:323-334; Meyerson et al., 1994, Mol. Cell. Biol. 14:2077-2086). Cdk-2 is activated primarily by association with cyclin E or cyclin A (Dulic et al., 1992, Science 257:1958-1961; Koff et al., 1992, Science 257:1689-1694; Rosenblatt et al., 1992, Proc. Natl. Acad. Sci. USA 89:2824-2828).

A connection between cdks and herpesvirus replication has been disclosed by Bresnahan et al. (1997, Virology 231:239-247), wherein inhibition of cytomegalovirus DNA replication was reported to be mediated by the cdk inhibitors, Roscovitine (Rosco) and Olomoucine (Olo). The authors suggest that the dependence of CMV replication on cell proliferation accounts for the inhibition of replication of this virus in the presence of compounds which inhibit cell proliferation.

The herpes simplex virus type 1 (HSV-1) genome is a linear double-stranded DNA molecule composed of two unique components, designated unique long (UL) and unique short (US), each of which is flanked by inverted repeat sequences (Hayward, et al., 1975, Proc. Natl. Acad. Sci. USA. 72:4243-4247; McGeoch et al., 1988, J. Gen. Virol. 69:1531-1574). The HSV-1 genome contains three origins of DNA replication, one located within UL (oriL) and two within the repeat sequences flanking US (oriS) (Challberg et al., 1989, Ann. Rev. Biochem. 58:671; Spaete et al., 1985, Proc. Natl. Acad. Sci. USA 82:694; Stow et al., Virology 130:427).

Expression of HSV-1 genes during productive infection proceeds in a coordinate and sequential manner (Honess et al., 1974, J. Virol. 14:8-19). The classification of HSV-1 proteins into broad groups, immediate-early (IE), early (E), delayed early (DE), and late (L), is based on the kinetics of synthesis of individual viral transcripts and proteins, the effects of various metabolic inhibitors on DNA, RNA and protein synthesis, and studies using viral mutants.

In contrast to the complex sequence of events which occurs during productive infection, in latently infected cells, viral gene expression is limited to the latency-associated transcripts (LATs). The LATs are a family of transcripts ranging in size from 2.0 to >8 kilobase pairs (kb) (Stevens et al., 1987, Science 235:1056-1059; Spivak et al., 1987, J. Virol. 61:3841-3847; Zwaagstra et al., 1990, J. Virol. 64:5019-5028). The factors which mediate the switch from productive infection to latency and vice versa are not known.

Given the frequency and often the severity of herpesvirus infection in humans, there is a great need to develop compounds which inhibit virus replication. To date, anti-herpesvirus therapeutics have been directed primarily at the inhibition of herpesvirus DNA replication, an event which occurs following the expression of immediate-early and early genes. Therapeutic compounds, which are directed to inhibition of herpesvirus immediate-early gene expression, should be superior to those that inhibit DNA replication. A compound able to inhibit herpesvirus immediate-early gene expression would inhibit virus replication at a stage prior to the de novo expression of all known viral genes (immediate-early, early and late genes) and consequently, prior to the generation of cytotoxic proteins, or to the production of any viral antigen that could stimulate the host immune system, or to the production of any progeny virus. Thus, inhibition of virus replication at the immediate-early stage has the added advantage of ensuring inhibition of the cytotoxic effects of the virus on cells, as well as the generation of progeny virus thereby preventing the spread of virus infection in the host. To date, with the exception of global inhibitors of transcription and translation, there are no available compounds, which specifically inhibit herpesvirus immediate-early viral gene expression. Thus, there is a long recognized need in the art for the identification and use of such compounds.

There is also a long felt need in the art for the identification and use of compounds which inhibit herpesvirus replication by inhibiting viral functions at various stages in the virus life cycle, including inhibition of virus replication at the level of expression of other classes of viral genes and inhibition of reactivation of virus from the latent state.

Compounds, which directly inhibit viral functions, such as those, involved in viral DNA replication have a significant disadvantage in that, viral proteins evolve which are resistant to the compound. Thus, the generation of virus strains, which are impervious to the antiviral compound, is a common problem in the development of anti-herpesviral compounds. There is therefore also a long recognized need in the art for the development of compounds, which do not induce the production of drug-resistant strains of virus.

Further, the host immune system induced in response to herpesvirus infection is a critical pathogenic mechanism in all diseases caused by herpesviruses. Consequently, there is a long recognized need in the art for the identification and use of compounds, which simultaneously inhibit herpesvirus replication and the immune response to the same herpesvirus infection in a host.

There is also a long felt need in the art for agents, which inhibit all microbial infections. Flores et al. (1999, Proc. Natl. Acad. Sci. USA 96:7208-7213) used three compounds that inhibit cdk (P-TEFb) to inhibit replication of Human Immunodeficiency Virus (HIV) replication. The authors conclude that cdk9 is required for HIV replication. This study does not address other cdk inhibitors as antimicrobial agents. Further, Chao et al. (2000, Journal of Biological Chemistry, Jul. 21, 2000) also states that HIV replication can be inhibited by inhibitors of cdk9. The present invention satisfies these needs. The present invention provides compositions and methods which inhibit microbial replication by inhibiting cdks other than cdk9.

There is a long felt need in the art for a drug which blocks replication of a pathogen by targeting proteins which are encoded by the host. This invention satisfies this need.

### BRIEF SUMMARY OF THE INVENTION

The invention relates to a method of identifying compounds which inhibit replication of drug-resistant pathogenic agents by inhibiting host-encoded cellular proteins.

The invention relates to a method of inhibiting the replication of a drug-resistant pathogenic agent, the method comprising contacting a cell comprising the drug-resistant pathogenic agent with a compound capable of inhibiting replication of the drug-resistant pathogenic agent, wherein the compound targets a cellular protein, thereby inhibiting replication of the drug-resistant pathogenic agent.

In one aspect, the drug-resistant pathogenic agent is selected from the group consisting of a virus, a bacterium, a fungus, a yeast and a parasite.

In one embodiment, the drug-resistant pathogenic agent is a virus.

In one aspect, the virus is selected from the group consisting of a herpesvirus, a hepatitis B virus, a hepatitis C virus, a human papilloma virus, human immunodeficiency virus (HIV) and human T-cell leukemia virus (HTLV).

In another aspect, the virus is HIV.

In yet another aspect, the virus is a herpesvirus.

And in yet another aspect, the herpesvirus is selected from the group consisting of herpes simplex virus type 1 (HSV-1), herpes simplex virus type 2 (HSV-2), cytomegalovirus, varicella zoster virus (VZV), bovine herpesvirus type 1 (BHV-1), equine herpesvirus type 1 (EHV-1), pseudorabiesvirus (PRV), Epstein Barr virus, human herpesvirus type 6, human herpesvirus type 7 and human herpesvirus type 8.

In one embodiment, the herpesvirus is HSV.

In one aspect, the herpesvirus is HSV-1.

In one embodiment, the compound is a cdk inhibitor.

In one aspect, the cdk inhibitor is selected from the group consisting of 6-dimethylaminopurine, isopentenyladeninne, olomoucine, roscovitine, CVT-313, purvalanol A&B, flavopiridol, suramin, 9-hydroxyellipticine, toyocamycin, staurosporine, γ-butyrolactone, CGP60474, kenpaullone, alsterpaullone, indirubin-3′-monoxime and hymenialdisine.

In another aspect, the cdk inhibitor is selected from the group consisting of roscovitine, olomoucine, and provalanol.

The invention also relates to a method wherein the compound is a non-cdk inhibitor.

The invention further relates to a method of inhibiting the replication of a drug-resistant pathogenic agent in a mammal, the method comprising administering to a mammal a therapeutically effective amount of a compound capable of inhibiting replication of a drug-resistant pathogenic agent, wherein the compound targets a cellular protein, thereby inhibiting replication of the drug-resistant pathogenic agent in a mammal.

In one aspect, the mammal is a human.

In one embodiment, the method comprises administering said compound via a route selected from the group consisting of oral, rectal, vaginal, parenteral, topical, pulmonary, intranasal, buccal, ophthalmic and intrathecal.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a series of graphs, comprising Panels A-E, illustrating concentrations of Roscovitine (Rosco) and Olomoucine (Olo) required to inhibit cell cycle progression in HEL and Vero cells. HEL (Panels A, C, and E) and Vero (panels B and D) cells. 3×10⁵ to 6×10⁵ cells were seeded in 35 mm dishes (350 to 600 cells/mm²) in the presence or absence of the indicated concentrations of Rosco (Panels A and B), Olo (Panels C and D), or Lova (Panel E). Because of the short duration of some of the biological effects of Olo, cultures treated with Olo required a medium change at 6 hours post-infection (hours pi). After 24 hours, cells were harvested by trypsinization and resuspended in Telford's reagent and cellular DNA content was determined by FACS analysis. The percentages of cells in specific phases of the cell cycle are plotted against the concentration of drug.

FIG. 2 is a series of graphs, comprising Panels A-C, illustrating concentrations of Rosco and Olo required to inhibit HSV-1 replication in HEL and Vero cells. HEL or Vero cells (2×10⁵) in 12-well plates (500 cells/mm²) were infected with 2.5 PFU of HSV-1 strain KOS per cell. One hour later, the inoculum was removed, the cells were washed twice with cold PBS, and medium containing the indicated concentrations of Rosco (Panel A), Olo (Panel B), or PAA (Panel C) was added. Cultures treated with Olo required a medium change at 6 hours pi. After 24 hours, cells were harvested and virus was titrated by standard plaque assay. Viral titers at 24 hours pi are plotted against the drug concentration. Note that the scales on the y axis differ between Panel B and Panels A and C. Each time point indicates the average and range of two experiments.

FIG. 3 is a series of graphs, comprising Panels A and B, depicting the fact that inhibition of HSV replication by Rosco is reversible. HEL (Panel A) or Vero (Panel B) cells (1.5×10⁵) in 12-well plates (400 cells/mm²) were infected with 2.5 PFU of HSV-1 strain KOS per cell. After adsorption for 1 hour at 37° C., the inoculum was removed, the cells were washed twice with cold PBS, and medium containing Rosco (40 μM for HEL, 100 μM for Vero) or control medium containing no drug was added. Infected cells were harvested at 0, 3, 6, 9, 12, 18, and 24 hours pi. At 24 hours pi, medium was removed from the remaining wells, cells were washed with cold PBS, and medium in Rosco-treated, infected cultures was either changed from Rosco-containing to control medium lacking Rosco (release) or back to Rosco-containing medium (no-release control). The medium in infected cultures lacking Rosco was replaced with fresh medium lacking Rosco (no-treatment control). This medium change at 24 hours pi is indicated by the arrows. Cells were harvested at the indicated times after the medium change, and virus was titrated by standard plaque assays. Viral titers are plotted against time post infection (where time zero is the time of virus addition to cultured cells).

FIG. 4 is a series of graphs, comprising Panels A and B, illustrating the fact that Lovastatin (Lova), staurosporine (Stan), iso-olomoucin (iso-Olo), and PD98059 did not inhibit HSV-1 replication. HEL cells (2×10⁵) in 12-well plates (500 cells/mm²) were infected with 2.5 PFU of HSV-1 KOS per cell. After adsorption for 1 hour at 37° C., the inoculum was removed, the cells were washed twice with cold PBS, and control medium lacking drugs or containing the indicated concentration of the indicated drug was added. The drug concentrations were 40 μM Rosco, 75 μM Olo or iso-Olo, 20 μM Lova, 5 ng of staurosporine per ml, and 70 μM PD98059. These concentrations of drugs had no toxic effects on uninfected HEL cells for at least 24 hour as determined by microscopic evaluation. Cultures treated with Olo required a medium change at 6 hours pi. At the indicated times post infection, cells were harvested, frozen, thawed, and sonicated and the virus was titrated by standard plaque assay. Viral titers are plotted against time post infection (where time zero is the time of virus addition to cultured cells). Each time point indicates the average and range of two experiments. Results are presented as two graphs comprising Panels A and B, for clarity; consequently, the no-treatment (Control) and Rosco-treated (Rosco) controls are shown in both panels.

FIG. 5 is a graph illustrating the absence of Rosco- or Olo-resistant variants of HSV after 11 passages in selective medium. HSV-1 strain KOS was passaged eleven time in medium containing either Olo, Rosco, or PAA as described in the examples presented herein. One thousand PFU of each passage was used to infect 10⁵ Vero cells in duplicate. One set of infected cells was treated with inhibitory concentrations of the selective drugs (100 μg of PAA/ml, 100 μM Rosco, or 150 μM Olo), while the other set was left untreated (medium without drug). Virus was harvested and titrated at 24 hours pi. The medium in cultures treated with Olo was changed at 6 hours pi. The percentage of resistant virus after each passage was calculated by using the formula: percent resistance=100×(PFU in the presence of selection drug/PFU in medium without drug) and is plotted against the passage number. The figure depicts the percentage of virus resistant to Olo, Rosco, or PAA.

FIG. 6 is an image of sections of gels following RNA protection assays depicting viral RNA accumulation in the presence and absence of Olo or Rosco. HEL cells (7.5×10⁵) in 60-mm dishes (275 cells/mm²) were infected with 2.5 PFU of HSV-1 per cell in the presence of 75 μM Olo (0) or 40 μM Rosco (R) or in the absence of a drug (C), and total (cellular and viral) RNA was extracted at 2, 5, 8, 12, or 15 hours pi, as well as from mock-infected cells (MI). Levels of GAPDH-, ICP4-, TK-, and ICP8-specific RNAs were measured by standard RNase protection assays (ProtectDirect: Ambion) as recommended by the manufacturer, with minor modifications.

FIG. 7 is a series of images of sections of gels following RNA protection assays depicting levels of viral immediate-early, early and late transcripts in the presence of Rosco added at 1, 2, or 6 hours pi. Vero cells were infected and the medium was changed at different times after infection. A series of infected monolayers were left in drug-free medium throughout the 18 hours of the experiment (Control). Immediately before (0), and at 1, 2, 6, 9, 12 and 18 hours pi, cells were harvested and viral and cellular RNA was extracted. RNA was also extracted from mock-infected cells as a negative control (MI). Levels of ICP0, ICP4 (both immediate-early), ICP8, TK (both early) and gC (late) transcripts were evaluated by RNase protection assays. Levels of GAPDH were also measured to ensure equal loading of samples. The apparent drop in the level of ICP8 mRNA at 12 hours pi in the control is a technical artifact not observed in repeat experiments.

FIG. 8 is a series of graphs depicting HSV replication in the presence of Rosco added after removal of CHX at 6 hours pi. FIG. 8A: Vero cells were pretreated with CHX for 1 hour, infected with 3 PFU/cell of HSV-1, washed, and overlaid with medium containing 50 μg/ml of CHX. At 6 hours pi, CHX-containing medium was removed, the cells were washed twice with PBS and fresh medium which did not contain any drug (C), or which contained 50 μg/ml CHX (CHX), or 100 μM of Rosco (RO) was added. The PBS used for the washes contained the same drugs at the same concentrations as the medium which was added to the respective cultures after washing. Twenty-four hours after the change of the medium, the cells were harvested and virus titers were measured in standard plaque assays. Each bar represents the average and range of two experiments. FIG. 8B: Vero cells were infected with 3 PFU/cell of HSV-1, washed, and overlaid with medium containing no drug (C), 50 μg/ml of CHX (CHX), or 100 μM of Rosco (RO). Cells infected in the presence of CHX had been pretreated with the same drug for 1 hour before infection. Twenty-four hours after infection, the cells were harvested and virus titers were measured in standard plaque assays. Each bar represents the average and range of two experiments.

FIG. 9 depicts levels of expression of viral immediate-early, early and late transcripts and proteins when Rosco was added after a 6 hour CHX block. FIG. 9A: Vero cells were infected with 2.5 PFU/cell of HSV-1, washed and overlaid with medium containing 50 μg/ml of CHX. At 6 hours pi, medium was removed from the infected wells, cells were washed twice with drug-containing PBS and fresh medium containing 50 μg/ml CHX (CHX), no drug (Control), or 100 μM Rosco (Rosco), was added. Immediately before (0), and at 3, 6, and 9 hours post-release (hpr) of the CHX block and addition of the secondary drug, cells were harvested and RNA was extracted. RNA was also extracted from mock-infected cells as a negative control (MI). Levels of ICP0, ICP4 (both immediate-early), ICP8, TK (both early) and gC (late) transcripts were evaluated by RNase protection. Levels of GAPDH were also measured to ensure equal loading from the different samples. FIG. 9B: Vero cells were infected with 6 PFU/cell of HSV-1 for 6 hours in the presence of CHX, followed by removal of the drug and incubation in drug-free medium (C) or in medium containing 100 μM of Rosco (RO). At the time of release, ³⁵S methionine was added to the cultures. For comparison, mock-infected cells were also releases and radioactively labeled for 6 hours (MI). At 6 (0-6 hpr) and 12 hours post release (0-12 hpr), the cells were harvested and proteins obtained therefrom were resolved in a discontinuous 6% polyacrylamide gel. The regions of the gel labeled from 0-6 hpr highlighted with vertical lines are shown expanded on the left of the figure, where the relevant IE proteins are indicated by arrows. The ratios beneath each protein designation designate the amount of the indicated protein synthesized in the presence of Rosco relative to the amount synthesized in drug free medium. Molecular weights, estimated from the mobility markers, are indicated between the two main gels (0-6 hpr and 0-12 hpr). On the right side of the gel labeled 12 hrp, solid arrowheads indicate IE proteins from top to bottom—ICP4, ICP0, ICP22 and ICP27, the latter which comigrate with a cellular protein in this concentration of polyacrylamide. ICP47 is not visible in these gels. The open arrowheads indicate the positions of E and L proteins.

FIG. 10 is a graph which depicts the kinetics of Rosco-dependent inhibition of IE promoter activation. Vero cells (2×10⁵ cells per 60 mm dish) were transfected with 1 μg pWRICP0-CAT and at 48 hours post transfection, the cultures were either mock-infected or were infected with 10 PFU/cell of UV inactivated KOS. The cultures were divided into 6 groups containing 6 dishes each. At 0, 2, 4 and 6 hours pi, the culture medium in a single group was removed and replaced with medium containing 100 μM Rosco. In addition, at 0, 2, 4, 6, 8, and 10 hours pi, one dish from each group was harvested and CAT activity expressed therein was measured. The mock-infected group was not treated with Rosco. CAT activity was measured in the linear range of the assay and a value of 40,000 cpm represents approximately 20% acetylation of the radiolabeled chloramphenicol substrate in the reaction mixtures.

FIG. 11 is a graph which depicts the effect of Rosco on HSV replication when the drug was added at 3 hour intervals after infection. Vero cells were infected with 2.5 PFU/cell of HSV-1. After 1 hour adsorption, cells were washed and overlaid with control medium containing no drug or with medium containing 100 μM Rosco. At 3, 6, 9, 12, 15, 18, or 21 hours pi, medium was removed and replaced with medium containing 100 μM Rosco (dotted lines, black triangles). Changes to Rosco-containing medium are indicated by the arrows. A second series of infected monolayers was left in drug-free medium (solid line, black squares). At 1, 3, 6, 9, 12, 18, 21, and 24 hours pi, cultures were harvested and viral titers determined by standard plaque assay. Viral titers were plotted as a function of time post infection (hours pi). Each time point indicates the average of two independent experiments.

FIG. 12 depicts the fact that inhibition of HSV replication by Rosco added at the time of release from a 12 hour PAA block is multiplicity dependent. FIG. 12A: Vero cells were infected with HSV-1 at the indicated multiplicities, washed and overlaid with medium containing 100 μg/ml of PAA or 100 μM Rosco. Infected monolayers were harvested at 24 hours pi, and viral replication was calculated by dividing the amount of virus present at 24 hour pi by the amount of virus adsorbed. FIG. 12B: Vero cells were infected with HSV-1 at the indicated multiplicities, washed and overlaid with medium containing 100 μg/ml of PAA. At 12 hours pi, medium was removed from the infected monolayers and was replaced with fresh medium containing no drug (PAA/C), 100 μg/ml PAA (PAA/PAA), or 100 μM Rosco (PAA/RO). Twenty-four hours after the change of medium, cells were harvested and viral titers were determined in a standard plaque assay. Viral yields at 24 hours pi were plotted against the multiplicities of infection. The dotted line indicates the PFU/10⁶ cells in the different inocula, presented for reference.

FIG. 13 depicts the fact that inhibition of HSV DNA replication by Rosco added at the time of release from a 12 hour PAA block is multiplicity-dependent. FIG. 13A: Vero cells were infected with the indicated multiplicities of HSV, washed and overlaid with medium containing 100 μg/ml of PAA. At 12 hours pi, cells were harvested, viral DNA was extracted and quantitated by slot blot analyses. After quantitation in a Molecular Dynamics PhosphorImager system, the fold-increase in viral DNA replication was calculated by dividing the amount of DNA detected at 12 hours post release by the amount of viral DNA detected immediately after infection and subtracting 1, such that a total block in viral DNA replication by the secondary drug is indicated by “0-fold-increase”. The fold increase in DNA replication was then plotted against the multiplicity of infection. FIG. 13B: Vero cells were infected and released from the PAA block as indicated above. Immediately before and twenty-four hours after the change of medium, cells were harvested and levels of viral DNA were determined by slot blot hybridization using a Molecular Dynamics PhosphorImager system. Fold-increase in viral DNA replication after release of the PAA block was calculated by dividing the amount of viral DNA detected at 24 hours post release by the amount of viral DNA detected immediately before release and subtracting 1, such that a total block in viral DNA replication by the secondary drug is indicated by “0-fold-increase”. The fold increase in DNA replication was then plotted against the multiplicity of infection. FIG. 13C: Vero cells were infected at the indicated multiplicities with HSV, washed and overlaid with medium containing 100 μg/ml of PAA. At 12 hours pi, medium was removed, and fresh medium containing no drug (PAA12-C 24), 100 μg/ml PAA (PAA 12-PAA 24), or 100 μM Rosco (PAA 12-Ro 24) was added. Twenty-four hours after the change of medium, cells were harvested and the amount of viral DNA in the cells was determined by slot blot analysis.

FIG. 14 is a series of images depicting steady-state levels of viral IE, E and L transcripts when Rosco was added to infected monolayers at the time of release of a 12 hour of PAA block. Vero cells were infected with 2.5 PFU/cell of HSV and the medium was replaced as described in the legend to FIG. 13. Immediately before (0), and at 4, 8, and 16 hours after release (hpr) from the PAA block and addition of the secondary drug, cells were harvested and RNA was extracted. RNA extracted from mock-infected cells served as a negative control (MI). Levels of ICP0, ICP4 (both IE) (FIG. 14A), ICP8, and TK (both E) (FIG. 14B) transcripts were evaluated by RNase protection assays. Levels of GAPDH were also measured to ensure equal loading of the samples.

FIG. 15 is a series of slot blots depicting inhibition of HSV DNA replication by Rosco with or without CHX added at the time of release from a 12 hour PAA block. Vero cells were infected with 2.5 PFU/cell of HSV-1, incubated in the presence of 100 μg/ml PAA for 12 hours and released from the block as described herein, except that CHX alone or together with the secondary drug was added to one set of infected monolayers at the time of release (CHX+). At 1 hour pi (−11 hours post release), immediately before release (0 hour post release; 12 hours pi), and at 12, 16 and 20 hours post release (24, 28 and 32 hours pi, respectively), cells were harvested and total DNA was extracted. Levels of viral DNA at the indicated times were analyzed by slot blot hybridization. Data from one of two qualitatively identical experiments is presented.

FIG. 16 is a series of graphs depicting inhibition of HSV DNA replication by Rosco added at the time of release from a 12 hour PAA block in the presence or absence of CHX. The images presented in FIG. 15 were quantitated using a PhosphorImager system, and the fold-increase in viral DNA replication after release from the PAA block was calculated by dividing the amounts of viral DNA at the indicated times post-release by the amounts of viral DNA before release and subtracting 1, such that total inhibition of DNA replication by the secondary drug is represented by “0-fold-increase”. Fold-increase in DNA replication was plotted against time after release (hpr).

FIG. 17 is a graph depicting replication of four HSV-1 ts mutants after shift-down from the non-permissive (39.5° C.) to the permissive (34° C.) temperature in the presence or absence of Rosco. Vero cells were infected at the non-permissive temperature with 2.5 PFU/cell of the indicated HSV-1 ts mutants. Six hours after infection, infected cultures were transferred to the permissive temperature in the presence of 100 μM Rosco or in the absence of drug (Control). One culture infected with each ts mutant was harvested immediately before shift-down. Twenty-four hours after shift-down, the remaining infected monolayers were harvested and viral replication was monitored by standard plaque assay. Fold viral replication after release was determined by dividing the titers at 24 hours post-release by the titers measured before release.

FIG. 18 depicts HSV DNA replication in the presence of Rosco added at the time of shift-down. FIG. 18A: Vero cells were infected with HSV ts A15 at the non-permissive temperature and released in the absence of drug or in the presence of 100 μM Rosco, or 100 or 400 μg/ml of PAA (Control, RO, PAA100 and PAA 400, respectively) as described in the text. One hour after infection (1 hour pi/−5 hours post release), immediately before release (6 hours pi/0 hours post release) and at 15 and 24 hours post release (21 or 30 hours pi, respectively), cells were harvested and DNA was extracted therefrom. DNA was also extracted from mock-infected cells for comparison (Mock). Levels of viral DNA replication were determined by slot blot analysis. For comparison, a set of infected monolayers was incubated at the permissive temperature throughout the experiment (34° C.). FIG. 18B: The slot blots shown in FIG. 18A were quantitated using the ImageQuant software package (Molecular Dynamics, California), and the fold-increase in DNA replication was calculated by dividing the amount of DNA at any given time point by the amount of DNA attained immediately before release and subtracting 1, such that complete inhibition of viral DNA replication after release is indicated by “0-fold-increase”. Fold-increase in DNA replication is plotted against time post release (hpr).

FIG. 19 is a series of images of gels depicting the fact that expression of E gene products was not inhibited by Rosco added at the time of shift-down. FIG. 19A: Vero cells were infected with ts Al 5 at the non-permissive temperature and shifted-down to the permissive temperature as described in the legend to FIG. 18. Immediately before release (Pre), or 16 hours post release in the absence of drug or in the presence of 100 μM Rosco, or 100 or 400 μg/ml of PAA (C, R0, P1, and P4, respectively), cells were harvested and RNA extracted. Steady-state levels of the transcripts of the genes encoding ICP8 or TK were evaluated by RNase protection assays. FIG. 19B: Vero cells were infected with ts A15 at the non-permissive temperature and shifted-down to the permissive temperature as described in FIG. 19A, except that methionine-free medium supplemented with ³⁵S-labeled methionine and the indicated drugs were added at the time of release. Infected cells were harvested at 16 or 24 hours post release, and viral proteins were resolved in a SDS-polyacrylamide gel. Molecular weights markers are indicated on the right. Late viral proteins (γ1 and γ2) are indicated on the left side of the gel with empty arrowheads. Viral IE and E proteins are indicated on the right side of the gel with black arrowheads. Except for ICP8 and TK, the ICP nomenclature was used, where VP16 is designated ICP25/26. The increase in overall intensity in the lanes C16 and C24 is due to the large amount of L proteins synthesized after shift-down in the absence of drugs. Note that the intensity of the bands corresponding to IE or E proteins did not change nearly as dramatically as the intensity of the bands corresponding to L proteins.

FIG. 20 is a series of graphs depicting the fact that topical treatment with Rosco inhibited HSV-1 replication in the eyes of HSV-1 infected ICR mice. Mice were infected, treated and their eyes swabbed as described herein. The control group consisted of 5 mice, and the treatment group consisted of 15 mice. FIG. 29A: Average titers of virus in eyes of all animals in each group and Standard Error of the Mean (SEM) are plotted against days pi. FIG. 29B: Percentage of mice positive for viral shedding, as measured by standard plaque assay, plotted against days post-infection.

FIG. 21 is a series of graphs depicting the fact that intramuscular (IM) administration of Rosco inhibited development of HSV-1 induced stromal keratitis in C.AL.20 mice. Mice were infected, treated and evaluated clinically as described in the text. Control and Rosco-treated groups consisted of 5 mice each. Scores of each eye are plotted against day pi. All clinical evaluations were performed in a blind manner. The dashed lines indicate the termination of treatment on day 11 pi, after the evening treatment. FIG. 21A depicts data obtained in vehicle treated animals and FIG. 21B depicts data obtained in Rosco treated animals.

FIG. 22 is a graph depicting the average corneal scores of HSV-infected mice. The scores (Table 4) of individual corneas presented in FIG. 21 were averaged. Mean scores of Control or Rosco-treated mice and SEM are plotted against day pi.

FIG. 23 is a series of images depicting histo-pathological examination of the corneas from all mice in the control group. At 36 days pi, mice were euthanized, and eyes were removed and processed for histopathological analysis by routine methods. The corneas were sectioned at different levels, stained and evaluated microscopically. Corneas from two uninfected A/J mice and from two HSV-1 infected ICR mice euthanized at 35 days pi were also processed for comparison (FIG. 23A, corneas A through D). Pictures of sections from the same level are shown for all corneas. Original magnification was 100× (FIG. 23A), or 40× (FIG. 23B).

FIG. 23A: Corneas A and B—corneas of two uninfected A/J mice; Corneas C and D—corneas of two HSV-infected ICR mice euthanized at 35 days pi; 1 through 10—corneas of HSV infected C.AL.20 mice euthanized at 35 days pi. FIG. 23B: 1 through 10—corneas of HSV infected C.AL.20 mice euthanized at 35 days pi.

FIG. 24 is a series of images depicting histo-pathological examination of the corneas from all mice in the treatment group. At 36 days pi, mice were euthanized, and corneas were removed and processed for histopathological analysis by routine methods. One cornea was lost during processing. The remaining corneas were sectioned at different levels, stained and evaluated microscopically. Corneas from two uninfected A/J mice and from two HSV-1 infected ICR mice euthanized at 35 days pi were also processed for comparison (FIG. 24A, corneas A through D). Images of sections from the same level are shown for all corneas. Original magnification was 400× (FIG. 24A), or 40× (FIG. 24B). FIG. 24A: A and B—corneas of two uninfected A/J mice.; C and D—corneas of two HSV-infected ICR mice euthanized at 35 days pi.; 1 through 9—corneas of HSV infected C.AL.20 mice treated with Rosco from day 1 to 11 pi and euthanized at 35 days pi. FIG. 24B: 1 through 9—corneas of HSV infected C.AL.20 mice treated with Rosco and euthanized at 35 days pi.

FIG. 25 is a graph depicting the fact that Rosco treatment minimized body weight losses during acute HSV infection. Mice were weighed individually immediately before infection and twice a week, on the same days clinical scores of corneas were evaluated after infection. Average and SEM body weight was plotted against days pi.

FIG. 26 is a series of graphs depicting induction of cdk-2 in trigeminal ganglion (TG) neurons during explant. Latently infected ICR mice were euthanized and TG were removed and processed as described herein. Commercially available antibodies specific for cdk-1, cdk-2, cdk-3, cdk-5 and cdk-7 were used to detect expression of these proteins in TG neurons at different days prior to and after explant. The percentage of neurons in which low or high levels of immunoreactivity specific for each protein was plotted against time post explant (in days).

FIG. 27 is a series of graphs depicting induction of cyclins A and E, but not B1, in TG neurons during explant. Latently infected ICR mice were euthanized and TG were removed and processed as described in the text. Commercially available antibodies specific for cyclins A, B1, and E were used to detect expression of these proteins in neurons of latently or mock-infected TG on sequential days post explant. The percentage of neurons expressing each protein was plotted against time post explant (in days). The top graph, included for comparison, shows the cdk-2 data presented in FIG. 26 plotted on a different scale.

FIG. 28 is a graph depicting Rosco-mediated inhibition of explant-induced reactivation of latent HSV-1. Latently infected ICR mice were euthanized and TG were removed and explanted as described in the text. The percentage of reactivated TG, as measured by detection of infectious HSV in the explant media, is plotted against time post explant, measured in days.

FIG. 29, comprising FIGS. 29A-F, is a series of graphs depicting the effect of cdk inhibitors on replication of HIV.

FIG. 30, comprising four panels (A-upper and lower; B-upper and lower), is a series of graphs comparing the effects of drugs which target cellular cdk proteins and drugs which target pathogens on drug-resistant and drug-sensitive pathogens. Six groups of Vero cells were infected with 3 plaque forming units (PFU) of one of six different drug-sensitive and drug-resistant strains of HSV-1 and HSV-2. The three drug-resistant strains included ACGr5 (acyclovir-resistant), dlPst1 TK-(acyclovir-resistant) and PAAr5 (phosphonoacetic acid-resistant). The three drug-sensitive strains used were KOS (HSV-1), 186 (HSV-2) and 333 (HSV-2). After one hour of adsorption, inocula were removed, cells were extensively washed with PBS and refed with fresh medium. The medium was supplemented or not with different concentrations of either of two cellular cdk inhibitors, Roscovitine (Rosco) or Purvalanol (Purv), or with either of two antiviral drugs, Acycloguanosine (“acyclovir”—ACV) or phosphonoacetic acid (PAA). The cellular cdk inhibitor Rosco was used at concentrations up to 100 μM (FIG. 30A, upper panel) and the cellular cdk inhibitor Purv was used at concentrations up to 30 μM (FIG. 30A, lower panel). The antiviral drug PAA was used at concentrations up to 300 μg/ml (FIG. 30B, upper panel) and the antiviral drug ACV was used at concentrations up to 200 μM (FIG. 30B, lower panel). Infected cells were harvested at 24 hours post-infection and viral replication during these 24 hours was monitored by standard plaque assays.

### DETAILED DESCRIPTION OF THE INVENTION

It has been discovered in the present invention that compounds which inhibit one or more cdks inhibit the growth/replication of any infectious agent whose genome either encodes its own cdk or which depends for its replication and/or growth on cellular cdks. It has also been discovered in the present invention that drugs which target cellular cdk proteins inhibit replication of pathogens, even pathogens that are resistant to conventional anti-pathogenic drugs. It has been discovered in the present invention that strains of herpesvirus which are resistant to the replication-inhibiting effects of the antiviral drugs acyclovir and phosphonoacetic acid are sensitive to the replication-inhibiting effects of drugs which target cellular proteins, namely the cdk inhibitors Roscovitine and Purvalanol.

It has also been discovered in the present invention that the viruses lymphocoriomeningitis virus (LCMV) and vaccinia virus, which encode their own transcription and DNA/RNA replication proteins, are not susceptible to replication inhibition by the cdk inhibitor Roscovitine at the concentrations tested.

It has been further been discovered in the present invention that the cellular cdk inhibitor Roscovitine inhibits phosphorylation of RNA PolII by cdk-7, but not by cdk-8. Moreover, in both mock and HSV-infected cells, a cdk inhibitor was found to bind the same spectrum of proteins. Thus, purine-derived cdk inhibitors block replication of HSV specifically, but do not target viral TK or Pol or bind to HSV proteins.

The invention encompasses a method of therapy comprising the use of a cellular protein inhibitor to inhibit replication of a pathogen which is resistant to an antipathogenic drug. The invention demonstrates the principal that a drug which targets a cellular protein inhibits replication of a pathogen that is resistant to a conventional antipathogenic drug.

The assay of the invention is useful for the identification of agents which inhibit replication of pathogens resistant to agents targeted toward pathogens, by targeting host-encoded proteins.

Inhibitors of cdk9 as a primary target, and/or which as a result of cdk9 inhibition, inhibit replication of a pathogenic agent, are excluded from the present invention.

It has thus been discovered in the present invention that two chemical compounds originally described as specific inhibitors of cellular cyclin-dependent kinases (cdks) inhibit the replication of two viruses, a neurotropic alphaherpesvirus, herpes simplex virus (HSV) and HIV. Exhaustive efforts to isolate spontaneous HSV mutants resistant to these drugs were unsuccessful, indicating that both drugs target multiple viral and/or cellular functions essential for viral replication. Thus, cellular cdks are required for herpesvirus replication and inhibitors of cellular cdks inhibit herpesvirus replication.

As the data presented herein establish, inhibition of cdks results in inhibition of HSV replication at various stages in the virus life cycle. Based upon the present invention, it has now been discovered that HSV replication is inhibited by these compounds at the level of HSV immediate early and early gene expression, viral DNA replication and reactivation of HSV from the latent state. Importantly also, one HSV-mediated immunopathologic disease, herpesvirus stromal keratitis (HSK) has been discovered in the present invention to be alleviated following treatment of an HSV-1 infected mammal with a cellular cdk inhibitor.

While the data presented herein are directed to inhibition of HSV-1 replication and of HSV-1 triggered pathogenesis, the invention should not be construed to be limited solely to inhibition of pathogenesis triggered by or pathogenesis of HSV-1. Rather, the invention should be construed to include inhibition of any herpesvirus (excluding cytomegalovirus) which is in fact inhibited by the inhibitors described herein. Thus, at a minimum, the invention should be construed to apply to all members of the alphaherpesviruses, including, but not limited to, HSV-1, HSV-2, varicella zoster virus (VZV), bovine herpes virus type 1 (BHV-1), equine herpesvirus type 1 (EHV-1), pseudorabiesvirus (PRV), and the like. In addition, the invention should be construed to apply to other herpesviruses, including, but not limited to Epstein Barr virus, and human herpesvirus types 6, 7, and 8, and any and all human and non-human herpesviruses which are either known or have not yet been discovered. While reference to HSV, or HSV-1 is made in the discussion which follows, the use of these terms should not be construed to limit the invention solely to these viruses. The preferred viruses for use in the methods of the invention are HSV-1, HSV-2 and VZV. The most preferred virus for use in the methods of the invention is HSV-1.

The invention should be further construed to include the use of cdk inhibitors for inhibition of replication of infectious agents, including viruses, bacteria, fungi, yeast and parasites (both protozoa and helminths). Preferred infectious agents include herpesviruses, hepatitis B virus, hepatitis C virus and human papillomavirus. Exemplified herein is the inhibition of HIV replication using cdk inhibitors. As noted elsewhere herein, suitable cdk inhibitors include those which may inhibit cdk9; however, inhibition of cdk9 is not the primary target of the cdk inhibitor and inhibition of replication of the infectious agent does not directly result from inhibition of cdk9 activity.

In the process of identifying the mechanism by which cdk inhibitors inhibit HSV replication, it has been discovered herein that transcription of the immediate-early genes of HSV is blocked in the presence of these inhibitors. It should be noted that all subsequent viral gene expression is dependent on the activities of immediate-early proteins. Hence, cdk inhibitors block expression of all classes of HSV genes. Furthermore, the data provided herein establish that cdk inhibitory compounds block immediate-early gene transcription by preventing activation of one or more specific protein(s) which are required for immediate-early transcription. In addition, the data provided herein suggest that this protein (or proteins) is cellular in origin and is activated by virion-associated proteins very early in infection and may itself be associated with whole virions.

To test the effects of cdk inhibitors on HSV gene expression, an assay has been designed and developed that can be used to identify novel compounds capable of inhibiting HSV immediate-early gene transcription (and as a consequence, compounds which are capable of inhibiting HSV replication) by blocking cdks, cdk-mediated activation of effector proteins, or by inhibiting the activities of these effector molecules.

The assay of the invention comprises the following steps. An expression plasmid comprising an HSV immediate-early promoter/regulatory region operably linked to a reporter gene is introduced into mammalian cells. At specific times after introduction of the expression plasmid into cells, HSV virion proteins are provided to the cells in trans. A test compound is added to the cells, either before, during or after addition of the plasmid and virion proteins to the cells. Cells which are not administered the test compound serve as controls. The level of expression of the reporter gene in the cells is assessed. A lower level of expression of the reporter gene in cells to which the test compound is administered, compared with the level of reporter gene in cells to which the test compound is not administered is an indication that the test compound is capable of inhibiting expression of immediate-early genes.

The test compound which is added to the cells is one which is already known, or is presently unknown and is shown in the assays described herein, to inhibit cellular cdk activity. The test compound is therefore referred to herein as having cdk inhibitory activity.

To detect cdk inhibitory activity of a compound, a preparation of a desired active cdk enzyme is incubated in an appropriate buffer, which buffer includes a phosphate donor and an appropriate substrate. Enzymes having cdk activity may be obtained from any mammalian cell, from starfish oocytes, or from insect cells which are infected with recombinant baculoviruses expressing cdks and cyclins. The enzymes are obtained from cells using any ordinary biochemical/immunological or molecular biological technology readily available in the art. Purification of the enzyme may be performed using biochemical methods (e.g., column chromatography, HPLC, FPLC, protein precipitation, etc.), by immunoprecipitation using relevant antibodies, or by affinity binding of the kinase to appropriate proteins such as, but not limited to, p13 and p19 or to the relevant domains of these proteins. Suitable substrates generally comprise partially purified protein substrates, completely purified protein substrates, or peptide substrates which are synthesized, translated in vitro, or which are obtained by cleavage of a longer peptide. Some common cdk-1 and cdk-2 substrates include histone H1, myelin basic protein, chemically defined peptides containing the S/TP consensus target sequence, or derivatives thereof. Commonly used incubation temperatures in cdk assays range from about 20° C. to about 37° C. and assays are generally incubated for about 10 to about 120 minutes. Reaction buffers useful for such assays include those which contain MgCl, a buffering salt such as HEPES, Tris or other salt, and may also contain chelating and protease inhibitory agents. A commonly used phosphate donor is ATP having a detectable label attached thereto. Following incubation, kinase activity is assessed by measuring the amount of phosphate incorporated into the substrate or removed from the donor. Phosphorylated substrate is detected using an appropriate assay designed to detect transfer of the label from the phosphate donor to the substrate and will vary depending upon the type of substrate and the type of label used.

Several HSV immediate-early promoter/regulatory sequences may be used in the assay of the invention, including but not limited to, the HSV ICP0, ICP4, ICP27, ICP22 and ICP47 promoter/regulatory sequences. Preferably, the promoter regulatory sequence is the HSV-1 ICP0 promoter/regulatory sequence.

The assay of the invention should not be construed to be limited to any particular reporter gene, there being a multitude of reporter genes which are known or are heretofore unknown which may be used in the assay. Several different reporter genes are described in the art, including for example, in Sambrook et al. (1989, Molecular Cloning: A Laboratory Manual, Cold Spring Harbor Laboratory, New York) and in Ausubel et al. (1997, Current Protocols in Molecular Biology, Green & Wiley, New York). Reporter genes include, but are not limited to, the CAT gene, the luciferase gene, GUS, the green fluorescent protein gene and a growth hormone gene. The preferred reporter gene is the bacterial CAT gene.

The assay of the invention is also not limited to any particular cell, in that, any cell into which herpesvirus immediate-early expression plasmids may be introduced are suitable for use in the assay. Preferred cells for use in the assay are Vero cells. Other cells include, but are not limited to, HEL, PC12, BHK-2, BT and MDBK cells, and any suitable primary mammalian cell which is capable of expressing an alphaherpesvirus protein.

The assay of the invention is also not limited to the manner in which plasmids and/or virion proteins are introduced into the cells. For example, expression plasmids may be introduced into cells using any ordinary transfection procedures such as those described in Sambrook et al. (1989, Molecular Cloning: A Laboratory Manual, Cold Spring Harbor Laboratory, New York) and in Ausubel et al. (1993, Current Protocols in Molecular Biology, Green & Wiley, New York). Virion proteins may be added to the cells by “infection” of transfected cells with UV-inactivated HSV. The use of inactivated virus ensures that among all of the HSV proteins produced during infection, only those present in virions will be introduced into transfected cells. In one embodiment of the assay of the invention, inactivated HSV virions are allowed to enter infected cells by natural means. In another embodiment, proteins in inactivated HSV virions may be induced to enter cells more efficiently by using “fusigenic” compounds such as polyethylene glycol (PEG) which promote fusion between the viral envelope and the host cell membrane. This alternative approach ensures that test compounds that might conceivably inhibit entry of inactivated HSV virions into cells will not score as false positives in the assay. In yet another embodiment, virion proteins are added to cells by way of plasmids expressing the same.

Virion proteins to be added to the cell, whether by addition of inactivated virions or by the addition to cells of plasmids expressing these proteins, include, but are not limited to, VP16, ICP4, VP22, UL13 and US3. It is preferred that inactivated virions or a plasmid which expressed VP 16 be added to the cells.

The test compound may be added to the cells at any time during the assay. However, preferably, following the introduction of virion associated proteins into the cells, the medium containing the UV-inactivated viral inoculum is removed, the infected cells are washed, and medium containing the test compound is added. At selected times following the introduction of virion proteins into the cells, the activity of the reporter gene product is measured. The addition to the cells of a test compound having cdk inhibitory activity that blocks HSV immediate-early gene expression should result in diminished activity of the reporter gene product compared with the level of activity of the reporter gene product in cells to which the test compound is not administered.

In a variation of the assay of the invention, more than one of the HSV immediate-early promoter/regulatory regions operably linked to the same or different reporter genes may be introduced into cells in order to perform the assay. Use of more than one immediate-early promoter/regulatory region will provide information on whether the test compound is specific for any particular HSV immediate-early promoter/regulatory region.

The assay of the invention and compounds identified thereby, have several different uses. HSV is an important human pathogen and no drug or vaccine is currently available which is capable of preventing infection with this virus or of preventing reactivation of the virus from latency. Compounds which are identified in the assay of the invention may be used to treat HSV infections, including but not limited to cold sores, genital herpes, HSV encephalitis, herpesvirus stromal keratitis, and the like. A list of other herpesvirus diseases which can be treated are provided herein in Table 1. In addition, cdk inhibitors may be used to control infections with other neurotropic alphaherpesviruses of humans (varicella) and veterinary importance.

TABLE 1
Clinical Syndromes Associated with Herpesviruses

| | | | Varicella- | Epstein- | | | | | |
|---|---|---|---|---|---|---|---|---|---|
| System: | HSV-1 | HSV-2 | Zoster | Barr | Cytomegalo | HHV6 | HHV7 | HHV8 | Simiae |
| Gingivostomatitis | + | + | | | | | | | + |
| Genital lesions | + | + | + | | | | | | + |
| Cutaneous lesions | + | + | + | | | | | | + |
| Keratoconjunctivitis | + | + | + | | | | | | + |
| Retinitis | + | | | + | | | | | |
| Esophagitis | + | + | + | + | | | | | |
| Pneumonitis | + | + | + | + | + | + | | | |
| Hepatitis | + | + | + | + | + | | | | |
| Myopericarditis | | | + | + | | | | | |
| Meningitis | | + | + | | | | | | |
| Encephalitis | + | + | + | + | + | + | | | + |
| Myelitis | + | + | + | + | + | | | | + |
| Erythema multiforme | + | + | + | | | | | | + |
| Other rash | | | | + | + | + | + | | |
| Arthritis | | | + | | + | | | | |
| Hemolytic anemia | | | + | + | + | | | | |
| Leukopenia | | | + | + | + | | | | |
| Thrombocytopenia | | | + | + | + | | | | |
| Mononucleosis | | | | + | + | + | | | |
| Lymphoma | | | | | + | | | | |
| Congenital infection | | | | | + | | | | |
| Kaposi's Sarcoma | | | | | | | | + | |
| Herpes Zoster | | | + | | | | | | |
| (shingles) | | | | | | | | | |
| Exacerbated clinical | + | + | + | + | + | + | + | + | + |
| Syndromes in | | | | | | | | | |
| immunosuppressed | | | | | | | | | |
| individuals | | | | | | | | | |

Currently available antiherpesvirus drugs target single virus-encoded functions. Consequently drug-resistant mutants are frequent in viral populations and are readily selected during prolonged drug treatment of patients. As stated herein, in the present study, efforts to isolate HSV mutants which are resistant to cdk inhibitors were been unsuccessful, indicating that these variants arise at extremely low frequencies, if at all, in drug-treated, HSV-infected cells. The data presented herein establish that the use of a cdk inhibitor for inhibition of HSV-1 effects multiple viral targets, that is, immediate-early and early gene expression are affected, as is viral DNA replication and virus reactivation. Each of these functions required cdk activity, but it is possible that the same cdk is not required in all situations. Thus, the inability to induce drug-resistant mutants likely can be ascribed to the fact multiple targets and proteins are involved thereby rendering impossible the generation of drug-resistant mutants. Another major advantage of inhibiting cdk activation of downstream effector molecules is that this is the only known means of preventing transcription of HSV immediate-early genes in vivo, thus completely inhibiting immediate-early, early, and all subsequent viral gene expression.

The assay of the invention provides a heretofore unknown simple assay for the identification of compounds having cdk inhibitory activity which are able to inhibit HSV immediate-early and early transcription and viral DNA replication. Thus, the assay is simpler than any currently used screening technique. Variations of these techniques may also be used to screen for drugs that inhibit other viral functions, such as early gene expression or HSV DNA replication, particularly given the data provided herein wherein it is established that compounds having cdk inhibitory activity also inhibit DNA replication and early gene expression of HSV-1. It is important to note that the assays provided herein can easily be automated, thus facilitating the screening of large numbers of compounds at any one time. An assay in which compounds which inhibit reactivation of a herpesvirus can be identified is also described herein. The assays provided herein may be used to identify compounds having cdk inhibitory activity in vivo.

The immediate-early assay just described is also useful for the identification of a cdk inhibitor per se, in that, compounds which are identified in the assay may represent heretofore unknown compounds having cdk inhibitory activity.

In addition, the immediate early assay just described, and the additional early gene and DNA replication assays described below, may be used to identify compounds having cdk inhibitory activity which also have anti-tumor activity. Cdk inhibitors are known in the art to possess anti-tumor activity (Sausville et al., 1999, Pharmacology and Therapeutics 82(2-3):285-292), and as such, any compound identified in the assays provided herein are likely anti-tumor compound candidates. The identification of an anti-tumor compound is therefore accomplished by following the assays provided herein to identify a compound having cdk inhibitory activity, and then subsequently testing the compound for anti-tumor activity in any known anti-tumor assay.

Similarly, the compounds which are identified in the assays presented herein may also be useful for the identification of compounds which suppress the immune response and therefore which may be useful not only as antiviral compounds, but also may be useful for the treatment of autoimmune disease. These compounds are identified by first performing any of the assays provided herein, and then by subsequently testing the identified compound for an activity known to affect autoimmune disease, using protocols available in the art.

The invention also includes an assay which is designed to identify a cdk inhibitor which inhibits herpesvirus early gene expression. Herpesvirus “early genes” are those which are expressed prior to viral DNA replication and which require expression of herpesvirus immediate-early genes for their expression. Typical HSV early genes include, but are not limited to, UL42, ICP8 and thymidine kinase. This assay is performed essentially in the same manner as that described for inhibition of immediate-early gene expression. Basically, an expression plasmid comprising an HSV early promoter/regulatory region operably linked to a reporter gene is introduced into mammalian cells. At specific times after introduction of the expression plasmid into cells, HSV immediate-early proteins are provided to the cells in trans. A test compound is added to the cells, either before, during or after addition of the plasmid and virion proteins to the cells. Cells which are not administered the test compound serve as controls. The level of expression of the reporter gene in the cells is assessed. A lower level of expression of the reporter gene in cells to which the test compound is administered, compared with the level of reporter gene in cells to which the test compound is not administered, is an indication that the test compound is capable of inhibiting expression of early genes. Similar to the manner in which the assay for immediate-early gene expression is conducted, in the present assay, the order in which the steps of the assay are performed and in which the components of the assay are added to the assay mixture is not important and may vary depending on the type of test compound being tested.

An assay which is designed to identify a cdk inhibitor which inhibits herpesvirus DNA replication is also performed essentially in the same manner as that described for inhibition of immediate-early gene expression. An example of a DNA replication assay is described in the experimental examples provided herein and another DNA replication assay is now described. DNA replication assays are well known in the art and are disclosed in U.S. Pat. Nos. 5,665,873 and 5,616,461. To perform such an assay, basically, a plasmid comprising an HSV origin of DNA replication is introduced into mammalian cells. At specific times after introduction of the plasmid into cells, HSV proteins essential for DNA replication are provided to the cells in trans. Suitable proteins include DNA polymerase (UL30), ICP8, UL29, helicase primase (UL5, 8 and 52), origin binding protein (UL9), and polymerase accessory factor (UL42). A DNA replication assay is also described in Challberg et al. (1989, Ann. Rev. Biochem. 58:671-717). In the assay, a test compound is added to the cells, either before, during or after addition of the plasmid and virion proteins to the cells. Cells which are not administered the test compound serve as controls. The level of replication of the plasmid containing the origin of DNA replication is measured in a standard HSV DNA replication assay. A lower level of DNA replication in cells to which the test compound is administered, compared with the level of DNA replication in cells to which the test compound is not administered, is an indication that the test compound is capable of inhibiting HSV DNA replication. Similar to the manner in which the assay for immediate-early gene expression is conducted, in the present assay, the order in which the steps of the assay are performed and in which the components of the assay are added to the assay mixture is not important and may vary depending on the type of test compound being tested.

An assay designed to identify a compound having cdk inhibitory activity, which compound inhibits reactivation of HSV is also provided in the present invention. In this assay, an appropriate strain of mouse, for example, ICR mice, is used. The mice are infected with a suitable dose of HSV, for example, 10⁵ PFU per eye, by an appropriate route, for example, instillation following corneal scarification. Thirty-five or more days pi, latent virus is induced to reactivate using appropriate stimuli. By way of example, the mice may be euthanized using standard procedures, and the trigeminal ganglia (TG) are surgically removed from the dead animals. The TG are placed in tissue culture plates in the presence of standard tissue culture medium, for example, D-MEM supplemented with fetal bovine serum (FBS) and antibiotic and antifungal agents as necessary, in the presence or absence of supporting cells. Alternatively, explanted TG may be digested with collagenase or any other appropriate enzyme or combination of enzymes, to generate single cell suspensions of TG. The TG cells are then seeded onto a tissue culture plate in tissue culture medium. Following explant of the TG, virus reactivation from TG may be further stimulated using heat stress, that is, by incubating the TG at 43° C. for about 3 hours, or drugs known to induce virus reactivation may be added to the cell cultures. In each instance, virus is induced to reactivate in the presence or absence of the test compound. Reactivation of virus is monitored by detection of infectious (reactivated) virus in the supernatant of explanted TG or cells obtained therefrom, by detecting the presence of viral proteins or nucleic acid, which proteins and nucleic acids are characteristic of the lytic virus life cycle. Viral DNA replication may also be assessed in the cultures. A lower level of reactivated virus in cultures to which the test compound is administered, compared with the level of reactivated virus in cultures to which the test compound is not administered, is an indication that the test compound is capable of inhibiting HSV reactivation. Similar to the manner in which the assay for immediate-early gene expression is conducted, in the present assay, the order in which the steps of the assay are performed and in which the components of the assay are added to the assay mixture is not important and may vary depending on the type of test compound being tested.

By the term “lower level of reactivation” as used in this assay, is meant that less total virus may be present in the cultures, or that the kinetics of reactivation of virus is slower in the cultures, when compared with the levels of either of these two parameters in identical but non-drug treated cultures.

A reactivation assay may also be performed on latently infected lumbosacral ganglia in the case of HSV-2, by following the directions described herein for an assay involving latently infected TG.

Although cdk inhibitors have been shown to block replication of a gammaherpesvirus, human cytomegalovirus (HCMV), immediate-early and early gene expression of HCMV were not inhibited by these drugs (Bresnahan et al., 1997, Virology 231:239-247). Furthermore, inhibition of HCMV replication by cdk inhibitors may well prove to be secondary to inhibition of cell-cycle progression of the infected cell, whilst inhibition of HSV replication is clearly not. Thus, the inhibition of HSV immediate-early and early gene expression by inhibitors of cellular cdk activities is unique to HSV and likely to other neurotropic alphaherpesviruses. In addition, although cdk inhibitors have been proposed as anti-HCMV drugs, the possibility of using drugs that block the activation of downstream inhibitor molecules was not considered by the authors of this report, and in fact the very existence of such downstream effectors was not considered in the literature reporting this observation. Moreover, inhibition of the immune response during HCMV infection would be an undesirable side effect of cdk inhibitor action in the treatment of HCMV-induced disease because of the dependence of the host on the immune response for virus clearance. In contrast, inhibition of the immune response is highly desirable in the treatment of many diseases induced by infection of other herpesviruses, especially the alphaherpesviruses. The data presented herein establish that compounds having cdk inhibitory activity suppress a pathology mediated by the host immune response to an alphaherpesvirus infection. Thus, Bresnahan et al. (1997, Virology 231:239-247) teaches away from the present invention. In addition, the data presented herein establish, for the first time, that compounds having cdk inhibitory activity are capable of inhibiting reactivation of herpesviruses. Further, although some tyrosine-kinase inhibitors have been shown to be modest inhibitors of HSV replication, this modest inhibition was shown to be mediated by inhibition of an unknown virus-encoded function(s), and did not involve inhibition of immediate-early and early gene expression, viral DNA replication and virus reactivation.

With respect to the host immune response to herpesvirus infection, as noted herein, many herpesvirus infections, including alphaherpesviruses, have as a significant component of their pathogenesis, an immunologic effect, in that, viral pathogenesis is dependent on, or is exacerbated by, the host immune response to the virus. In other words, stimulation of the host immune response to virus infection, is responsible for or at the least, enhances the pathogenicity of the virus. In the present invention, it has been discovered that compounds having cdk inhibitory activity concomitantly suppress a pathology resulting from the host immune response to infection. Current treatments for herpesvirus infections which have such an immunopathogenic effect include the administration of an antiviral compound and an immunosuppressive compound. The present invention provides a treatment for such herpesvirus infections wherein a single compound having cdk inhibitory activity, is capable of inhibiting virus and of suppressing the host immune response, thereby eliminating the need for the use of additional combination therapies. However, it is important to note that the aforementioned discussion should be in no way construed as limiting the invention to the use of single drug therapies. Combination therapies are contemplated as part of the methods and compositions of the invention within the framework of being aware, as a result of the present invention, that compounds having cdk inhibitory activity are capable of dually inhibiting virus and of suppressing the host immune response.

The assay of the invention is therefore useful for the identification of novel antiviral agents capable of inhibiting herpesvirus replication.

Compounds which are identified in any of the assays described herein may be further tested for in vivo efficacy with respect to virus inhibition by following any of the protocols provided herein, or using protocols for assessing herpesvirus replication and/or reactivation in vivo which are available in the art.

Compounds which are identified in the assay of the invention may be administered to an animal, preferably a human, for the purpose of treating disease resulting from a herpesvirus infection therein. The invention thus also encompasses the use of pharmaceutical compositions of an antiherpesviral agent having cdk inhibitory activity, wherein the antiherpesviral agent is suspended in a pharmaceutically-acceptable carrier.

The invention should also be construed to include a method of inhibiting herpesvirus replication, including herpesvirus immediate-early and early gene expression, and herpesvirus DNA replication, which method includes inhibition of the replication of any herpesvirus, other than CMV, in a cell comprising administering to the cell a compound having cdk inhibitory activity. Preferred inhibitors are those which specifically target cdk-1, cdk-2, cdk-3, cdk-5 and cdk-7.

The cell in which herpesvirus replication is inhibited may be a cell in culture in the laboratory, such as a tissue culture cell, or a cell which is resident in a mammal, the mammal being preferably but not exclusively, a human.

The invention should also be construed to include a method of inhibiting herpesvirus reactivation, including any herpesvirus other than CMV, from a latent state in a cell comprising administering to the cell a cellular cdk inhibitor. Preferred inhibitors are those which specifically target cdk-1, cdk-2, cdk-3, cdk-5 and cdk-7.

The cell in which herpesvirus reactivation is inhibited may be a cell in culture in the laboratory, such as a tissue culture cell, or a cell which is resident in a mammal, the mammal being preferably but not exclusively, a human. Preferably, the cell is a neuronal cell, the herpesvirus is HSV and the mammal is a human.

While the examples provided herein are directed to the use of Rosco and Olo as preferred cdk inhibitors, these inhibitors being the preferred cdk inhibitors of the invention, the invention should not be construed to be limited solely to these inhibitors. Rather, the invention should be construed to include any and all compounds having cdk inhibitory activity as defined herein, which cdk inhibitory activity results in inhibition of herpesvirus replication and or inhibition of herpesvirus reactivation. Thus, the invention should be construed to include inhibitors which are identified in the assay of the invention provided herein, and to include other compounds which are either known or are as yet unknown, which exhibit cdk inhibitory activity. Thus, chemical modification of Rosco or Olo, which chemically modified Rosco or Olo compounds exhibit cdk inhibitory activity are also included in the invention. Other compounds which are useful in the methods of the invention include purvalanol, flavopiridol, peptides such as P21, P27, P57, peptidomimetics and dominant negative cdk mutants.

Compounds which are useful in any of the methods described herein may be formulated and administered to a mammal for treatment of a herpesvirus infection are now described.

The invention encompasses the preparation and use of pharmaceutical compositions comprising a compound useful for treatment of a herpesvirus infection as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “pharmaceutically acceptable carrier” means a chemical composition with which the active ingredient may be combined and which, following the combination, can be used to administer the active ingredient to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs, birds including commercially relevant birds such as chickens, ducks, geese, and turkeys, fish including farm-raised fish and aquarium fish, crustaceans and molluscs, such as farm-raised shellfish.

Pharmaceutical compositions that are useful in the methods of the invention may be prepared, packaged, or sold in formulations suitable for oral, rectal, vaginal, parenteral, topical, pulmonary, intranasal, buccal, ophthalmic, intrathecal, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents. Particularly contemplated additional agents include anti-emetics and scavengers such as cyanide and cyanate scavengers.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

As used herein, an “oily” liquid is one which comprises a carbon-containing liquid molecule and which exhibits a less polar character than water.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically-controlled release tablets. Tablets may further comprise a sweetening agent, a flavoring agent, a coloring agent, a preservative, or some combination of these in order to provide a pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e. about 20° C.) and which is liquid at the rectal temperature of the subject (i.e. about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or gel or cream or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulnonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nares.

Formulations suitable for nasal administration may, for example, comprise from about as little as 0. 1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

Typically, dosages of the compound of the invention which may be administered to an animal, preferably a human, range in amount from 1 μg to about 100 mg per kilogram of body weight of the animal. The precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even lees frequently, such as once every several months or even once a year or less. The frequency of the dose will be readily apparent to the skilled artisan and will depend upon any number of factors, such as, but not limited to, the type and severity of the disease being treated, the type and age of the animal, etc.

Diseases which can be treated using a compound having cdk inhibitory activity include, but are not limited to those listed in Table 1 provided herein. The invention should therefore also be construed to include methods of treating any of the aforementioned herpesvirus-associated diseases, wherein the method comprises administering to the mammal having the disease, a compound having cdk inhibitory activity in a therapeutically effective amount, wherein the compound is suspended in a suitable pharmaceutically acceptable carrier.

The invention also includes a kit comprising a compound having cdk inhibitory activity and an instructional material which describes adventitially administering the composition to a cell or a tissue of a mammal. In another embodiment, this kit comprises a (preferably sterile) solvent suitable for dissolving or suspending the composition of the invention prior to administering the compound to the mammal.

Definitions

The articles “a” and “an” are used herein to refer to one or to more than one (i.e. to at least one) of the grammatical object of the article. By way of example, “an element” means one element or more than one element.

“Plurality” means at least two.

As used herein, the term “antiviral agent” means a composition of matter which, when delivered to a cell, is capable of preventing replication of a virus in the cell, preventing infection of the cell by a virus, or reversing a physiological effect of infection of the cell by a virus. Antiviral agents are well known and described in the literature. By way of example, AZT (zidovudine, Retrovir® Glaxo Wellcome Inc., Research Triangle Park, N.C.) is an antiviral agent which is thought to prevent replication of HIV in human cells.

By the term “cdk inhibitory activity” as used herein when referring to a compound, is meant a compound which is capable of inhibiting cdk enzyme activity, or which is capable of inhibiting the effects of cdk enzyme activity. A cdk enzyme, when properly activated, usually serves to induce a cascade of events which ultimately mediate progression of the cell into different phases of the cell cycle. Thus, inhibition of the effects of cdk enzyme activity should be construed to include inhibition of one or more of the factors involved in this cell cycle cascade or any other signalling pathway regulated by the cdk inhibitor. Preferably, the cdk inhibitory compound of the invention has antiviral activity, and more preferably, the cdk inhibitory compound of the invention has both antiviral activity and is capable of suppressing the host immune response. The term “compound having cdk inhibitory activity” is used synonymously herein with the term “cdk inhibitor.”

The term “parasite” includes a protozoan and a helminth.

An “essential function” of a pathogen is one which is required for its DNA replication, RNA transcription, RNA processing, protein synthesis, protein processing or protein activity, or one which is required for the pathogen to elicit its pathogenic effects.

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

A “test” cell is a cell being examined.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, are reduced.

As used herein, an “instructional material” includes a publication, a recording, a diagram, or any other medium of expression which can be used to communicate the usefulness of the composition of the invention for its designated use. The instructional material of the kit of the invention may, for example, be affixed to a container which contains the composition or be shipped together with a container which contains the composition. Alternatively, the instructional material may be shipped separately from the container with the intention that the instructional material and the composition be used cooperatively by the recipient.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, the term “reporter gene” means a gene, the expression of which can be detected using a known method. By way of example, the E. coli lacZ gene may be used as a reporter gene in a medium because expression of the lacZ gene can be detected using known methods by adding the chromogenic substrate o-nitrophenyl-β-galactosidase to the medium (Gerhardt et al., eds., 1994, Methods for General and Molecular Bacteriology, American Society for Microbiology, Washington, D.C., p. 574).

A “subject” of diagnosis or treatment is a mammal, including a human.

A “prophylactic” treatment is a treatment administered to a subject who does not exhibit signs of a disease or exhibits only early signs of the disease for the purpose of decreasing the risk of developing pathology associated with the disease.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

The invention is now described with reference to the following Examples. These Examples are provided for the purpose of illustration only and the invention should in no way be construed as being limited to these Examples, but rather should be construed to encompass any and all variations which become evident as a result of the teaching provided herein.

### EXAMPLE 1

### Requirement for Cellular Cyclin-Dependent Kinases in Herpes Simplex Virus Replication and Transcription

The information presented in this Example may be summarized as follows. Several observations indicate that late G1/S phase specific cellular functions may be required for herpes simplex virus (HSV) replication: i) certain mutant HSV strains are replication-impaired during infection of cells in G0/G1 but not in G1/S phases of the cell-cycle, ii) several late G1/S phase-specific cellular proteins and functions are induced during infection, and iii) the activity of a cellular protein essential for expression of viral immediate-early genes, HCF, is normally required during the late G1/S phase of the cell-cycle. To test the hypothesis that late G1/S phase-specific cellular functions are necessary for HSV replication, HEL or Vero cells were infected in the presence of the cell-cycle inhibitors Roscovitine (Rosco) and Olomoucine (Olo). Both drugs inhibit cyclin-dependent kinases (cdks)-1 and -2 (required for cell-cycle progression into the late G1/S phase of the cell-cycle), and cdk-5 (inactive in cycling cells), but not cdk-4 or 6 (active at early G1). It was discovered that HSV replication was inhibited by Rosco and Olo, but not by Lovastatin (a cell-cycle inhibitor that does not inhibit cdk activity), Staurosporine (a broad-spectrum protein serine-threonine kinase inhibitor which does not inhibit cdks in vivo), PD98059 (an inhibitor specific for erk-1 and -2) or IsoOlomoucine (a structural isomer of Olo that does not inhibit cdk activity). The concentrations of Rosco and Olo required to inhibit cell-cycle progression and viral replication in both HEL and Vero cells were similar. Inhibition of viral replication was found not to be mediated by drug-induced cytotoxicity. Efforts to isolate Rosco or Olo resistant HSV mutants were unsuccessful, indicating that these drugs do not act by inhibiting a single viral target. Viral DNA replication and accumulation of immediate-early and early viral RNAs were inhibited in the presence of cell-cycle inhibitory concentrations of Rosco or Olo. It was therefore concluded that one or more cdks normally active from late G1 onward, or inactive in non-neuronal cells, are required for accumulation of HSV transcripts, viral DNA replication and production of infectious virus.

In mammalian cells, the nuclear environment varies considerably during each phase of the cell cycle. Thus, only S-phase nuclei contain all the transcriptional, enzymatic, structural and metabolic factors required for semi-conservative DNA replication (DePamphilis, 1996, DNA replication in eukaryotic cells, Cold Spring Harbor Laboratory Press, New York). To ensure the replication of their genomes, DNA-containing viruses have developed unique strategies to overcome the problems presented by a changing nuclear environment (Knipe, 1996, In: Fields Virology, 3 ed 2:1997-2025; DePamphilis, 1996, DNA replication in eukaryotic cells, Cold Spring Harbor Laboratory Press, New York). The simplest strategy is characteristic of the smallest DNA viruses, the parvoviruses, which replicate their genomes only when the infected cell progresses into S phase (Berns, 1996, Virology, 3 ed, 2:1997-2025; Knipe, 1996, In: Fields Virology, 3 ed 2:1997-2025; DePamphilis, 1996, DNA replication in eukaryotic cells, Cold Spring Harbor Laboratory Press, New York). The polyomaviruses (including SV40), on the other hand, induce infected cells to progress into S-phase (Cole 1996, Virology, 3 ed, 2:1997-2025; Knipe, 1996, In: Fields Virology, 3 ed 2:1997-2025; DePamphilis, 1996, DNA replication in eukaryotic cells, Cold Spring Harbor Laboratory Press, New York). Thus, these small DNA viruses are able to utilize cellular factors present or active in late G1 or early S as a consequence of either spontaneous or induced cell-cycle progression. Although these replication strategies are highly successful, support of viral replication is limited to those cells that are able to progress into S-phase. In contrast to these viruses, the alphaherpesviruses, such as herpes simplex virus (HSV), have adopted a strategy that permits genome replication in growth-arrested cells, including terminally differentiated non-cycling neurons, as well as in actively dividing cells. In this sense, HSV replication is cell-cycle independent. However, this does not imply that cellular function(s) associated with cell-cycle progression are not required for HSV replication. Indeed, relationships between HSV infection and cell-cycle related cellular functions are well-documented. Thus, HSV replication is blocked at the non-permissive temperature in five temperature-sensitive cell lines growth-arrested in G0/G1 (Yanagi et al., 1978, J. Virol. 25:42-50; Umene et al., 1996, J. Virol. 70:9008-9012). Moreover, HSV has long been known to replicate more efficiently in actively dividing than in growth-arrested cells of most types, and this enhancement in replication efficiency is especially prominent for certain HSV strains with mutations in genes not absolutely required for viral replication (Cai et al., 1991, J. Virol. 65:4078-4090; Daksis et al.,1992, Virology 189:196-202). For example, the replication impairment of ICP0-mutants can be complemented by cellular functions which are active during progression from G0 to the late G1/S phases of the cell-cycle (Cai et al., 1991, J. Virol. 65:4078-4090). Such complementation is consistent with a model in which during wild-type virus infection, ICP0 substitutes for or induces a cellular activity normally expressed only in the G1 and early S phases of the cell-cycle. In a similar vein, HSV mutants that do not express active thymidine-kinase (TK) or ribonucleotide reductase (RR) are impaired for replication in growth-arrested G0/G1 cells but replicate to wild-type levels in growing cells, which express the cellular counterparts of these viral enzymes in late G1/S (Goldstein et al., 1988, Virology 166:41-51; Jamieson et al., 1974, J. Gen. Virol. 24:465-480). At the molecular level, cellular proteins normally expressed only in late G1 and S (PCNA, RP-A, DNA Pol α, and DNA ligase 1), or directly involved in cell-cycle regulation (pRb and p53) have been detected in HSV DNA replication compartments of serum-starved cells, which are presumably arrested in G0/G1 (Wilcock et al., 1991, Nature 349:429-431). E2F DNA binding activity, cyclin-dependent kinase (cdk)-2 activity and cyclin A protein, which are all specific for late G1, S or G2 phases of the cell-cycle, have been reported to be induced during HSV infection of serum-starved cells (Hilton et al., 1995, Virology 213:624-638; Hossain et al., 1997, J. Gen. Virol. 78:3341-3348). Cyclin D3 has been reported to interact with ICP0 in vitro, and in vivo when the cyclin was expressed ectopically from the genome of the infecting virus (Kawaguchi et al., 1997, J. Virol. 71:(10):7328-7336). A cellular protein required for HSV immediate-early gene expression, HCF, has recently been shown to be an important cell-cycle regulator (Goto et al., 1997, Genes & Development 11:726-737).

The experiments conducted herein were performed to test the hypothesis that cell-cycle related factors normally active in uninfected cells in late G1 or early S phases (before the onset of cellular DNA synthesis) are required for HSV replication. If such factors are indeed required, inhibition of their activities should block viral replication. To test this hypothesis, the effects on viral replication of inhibitors of several cdks including those cdks whose activities are absolutely required for progression into late G1 and beyond (van den Heuvel et al., 1993, Science 262:2050-2054) were measured. Two such inhibitors have recently been described: Olomoucine (Olo) and Roscovitine (Rosco) (Meijer et al., 1997, Eur. J. Biochem. 243:527-536; Vesely et al., 1994, Eur. J. Biochem. 224:771-786). Both are purine-derivatives and display similar inhibitory profiles. Olo inhibits cdk-1/cyclin B, cdk-2/cyclin A or E, and cdk-5/p25. With the exception of extracellular receptor-activated kinases (erks) 1 and 2 (which are inhibited at about 10-fold higher concentrations than the cdk targets), Olo failed to inhibit 35 other enzymes tested, including protein serine/threonine (S/T) or tyro sine (Y) kinases, phosphatases, topoisomerases, DNA polymerases and a nucleo side kinase (Vesely et al., 1994, Eur. J. Biochem. 224:771-786). Rosco also inhibits cdk-1/cyclin B, cdk-2/cyclin A or E and cdk-5/p25 and, at greater than 20 fold higher concentration, erk-1 and 2, but not 25 other kinases (Meijer et al., 1997, Eur. J. Biochem. 243:527-536). Neither Olo nor Rosco has been reported to significantly inhibit the kinase activity of cdk-4 or -6, whereas the effects of these drugs on cdk-3, -7 and -8 have not been examined (Meijer et al., 1997, Eur. J. Biochem. 243:527-536; Vesely et al., 1994, Eur. J. Biochem. 224:771-786). Thus, both drugs inhibit cdks that are active, and whose activity is required, from late G1 onwards (van den Heuvel et al., 1993, Science 262:2050-2054). Consequently, Rosco and Olo block cell-cycle progression both in late G1/early S (when cdk-2 is required prior to the onset of cellular DNA synthesis) and in M (when cdk-1 is required for cell division) in a wide variety of mammalian cells, with an average IC₅₀ of 16.0 μM for Rosco and 60.3 μM for Olo (Vesely et al., 1994, Eur. J. Biochem. 224:771-786; Abraham et al., 1995, Biol. of the Cell 83:105-120; Glab et al., 1994 FEBS Letters, 353:207-211; Meijer et al., 1997, Eur. J. Biochem. 243:527-536; Iseki et al., 1997, Surgery 122:187-194; Graves et al., 1997, Anal. Biochem. 248:251-257. An important distinction between the two drugs is that Rosco blocks selected biological effects whereas Olo only delays them (Meijer et al., 1997, Eur. J. Biochem. 243:527-536; Vesely et al., 1994, Eur. J. Biochem. 224:771-786).

In this Example, data are provided which establish that both Rosco and Olo inhibit replication of HSV-1. The inhibitory effects are mediated by inhibition of cellular cdk activity and not of (a) viral protein in that: i) concentrations of Rosco and Olo that inhibit viral replication are proportional to the dose of each drug that inhibits cdk activity in vitro; ii) the concentrations of Rosco and Olo that inhibit viral replication are similar to those that block cell-cycle progression in two cell types (Vero and HEL); iii) neither Rosco nor Olo-resistant mutants were detected after extensive passage in the presence of the drugs; iv) inhibitors of cellular kinases (other than cdks) and inhibitors of cell-cycle progression that do not block cdks do not block viral replication; and v) a structural isomer of Olo that does not inhibit cdk activity does not inhibit HSV replication. Efforts to determine the level of inhibition of viral replication demonstrated that viral DNA replication was significantly reduced in the presence of either drug and, unexpectedly, the accumulation of immediate-early transcripts was also reduced as early as one hour after adsorption. These results strongly suggest the involvement of (a) cellular cdk(s) in the replication of a DNA containing virus that is capable of replicating in non-cycling cells. They also suggest the involvement of a cellular cdk in transcription of viral genes.

The Materials and Methods used in the experiments presented in this Example are now described.

Cells, Virus, Plasmids and Infections

Methods used for the propagation and maintenance of HEL and Vero cells have been described previously (Schaffer et al., 1973, Virology 52:57-71). A plaque purified low passage (p9) stock of HSV-1 strain KOS was used throughout these studies and prepared as described (Schaffer et al., 1973, Virology 52:57-71). The construction of plasmids prpTK, prp8 and prp4 has been described (Jordan et al., 1997, J. Virol. 71:6850-6862).

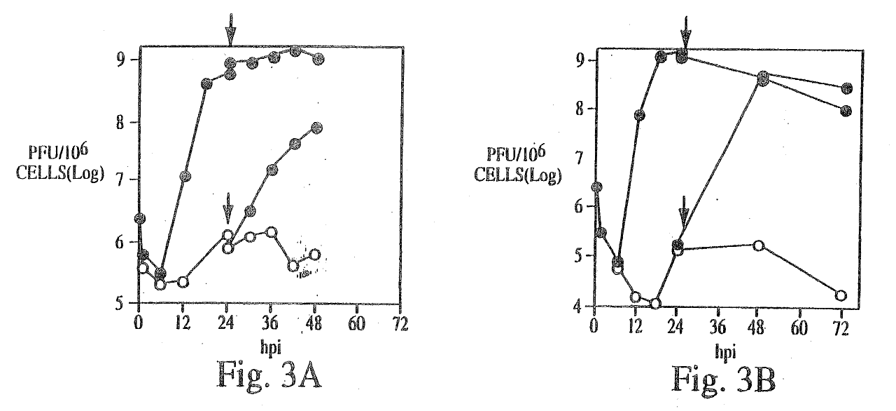

For infection of Vero or HEL cells, 1 to 5×10⁵ cells were infected with 2.5 to 3.0 PFU per cell of virus diluted in serum-free medium. After adsorption for 1 hour at 37° C., the viral inoculum was removed, monolayers were washed twice with cold PBS, and standard medium or medium containing the indicated drugs was added. In vivo, Olo does not block cdks completely, and its effects are relatively short-lived (Vesely et al., 1994, Eur. J. Biochem. 224:771-786; Meijer et al., 1997, Eur. J. Biochem. 243:527-536; Graves et al., 1997, Anal. Biochem. 248:251-257). Thus, for infections in the presence of Olo medium was replaced 6 hours after infection with an equal volume of fresh Olo-containing media. Changing medium at 6 hours post-infection (hours pi) reproducibly had no effect on the efficiency of HSV-1 replication. Infected cells were scraped into the media at the indicated times after infection (where T=0 is the time of addition of inoculum), and the total volume was transferred to a 5.0 ml tube and frozen at −70° C. After thawing, cells were sonicated for 45 seconds, and infectious virus was titrated by standard plaque assay. For the drug-release experiments described in FIG. 3, samples were harvested at the indicated times from 0 to 48 hours pi. At 24 hours pi, control or Rosco-containing (40 μM for HEL or 100 μM for Vero cells) medium was removed from infected monolayers. After washing monolayers twice with cold PBS to remove residual drug-containing medium, two volumes of control medium, or fresh medium containing the same concentration of Rosco, were added to each monolayer. Two volumes of medium were used to dilute any residual drug remaining on the monolayers after the washes.

Preparation of Drugs

Olo was purchased from Promega (Madison, Wis.); Rosco and Iso-Olomoucine were purchased from Calbiochem (San Diego, Calif.). The stock solutions of these three drugs were 100 mM in DMSO. The stocks of Staurosporine (Sigma, St. Louis, Mo.) and PD98059 (Calbiochem) were also diluted in DMSO, to a final concentration of 100 μg/ml and 20 mM, respectively. Lovastatin was obtained from Merck & Co., Rahaway, N.J., and was converted to its active lactonic form as described (Keyomarsi et al., 1991, Cancer Research 51:3602-3609), except that the final concentration of the stock solution was 10 mM. Phosphonoacetic acid (PAA) was purchased from Sigma, diluted in PBS, neutralized with NaOH, and further diluted with D-MEM to a stock concentration of 100 mg/ml. Stocks of all drugs were aliquoted and kept at −20° C. until use. Final dilutions of drugs in D-MEM containing 10% fetal bovine serum (FBS) were prepared immediately before use in the same batch of medium used in no-drug control infections. Except for PD98059, stocks were diluted at least 1:1000 to obtain the working concentrations of all other used drugs. The final concentration of each drug used is indicated in each figure.

FACS Analysis

2 to 6×10⁵ Vero or HEL cells were seeded in 35 mm dishes in 3.0 ml of media containing the indicated concentrations of Rosco, Olo or Lova. Twenty-four hours later, medium was removed and cells were washed with cold PBS. Cells were then treated with 200 μl of trypsin solution and resuspended with 1.8 ml of D-MEM-10% FBS. After centrifuging at 800× g for 10 minutes, cells were washed with 2.0 ml cold PBS, centrifuged again and resuspended in 3.0 ml of 70% ethanol. After fixation on ice for approximately 45 minutes, cells were centrifuged as above and resuspended in Telford's reagent (90 mM EDTA, 2.5 mU/ml RNAse A, 50 μg/ml propidium iodide, and 0.1% Triton X-100 in PBS) to a final concentration of 1.0×10⁶ cells/ml. Following incubation in an ice bath for approximately 2 hours, total DNA content was analyzed in a FACSCalibur analyzer using CellQuest software (Becton Dickinson, San Jose, Calif.). Cells were gated by forward scattering (FL-W), to avoid analyzing cell doublets, and limits to G1, S and G2+M cells were set manually.

Selection of Viral Mutants

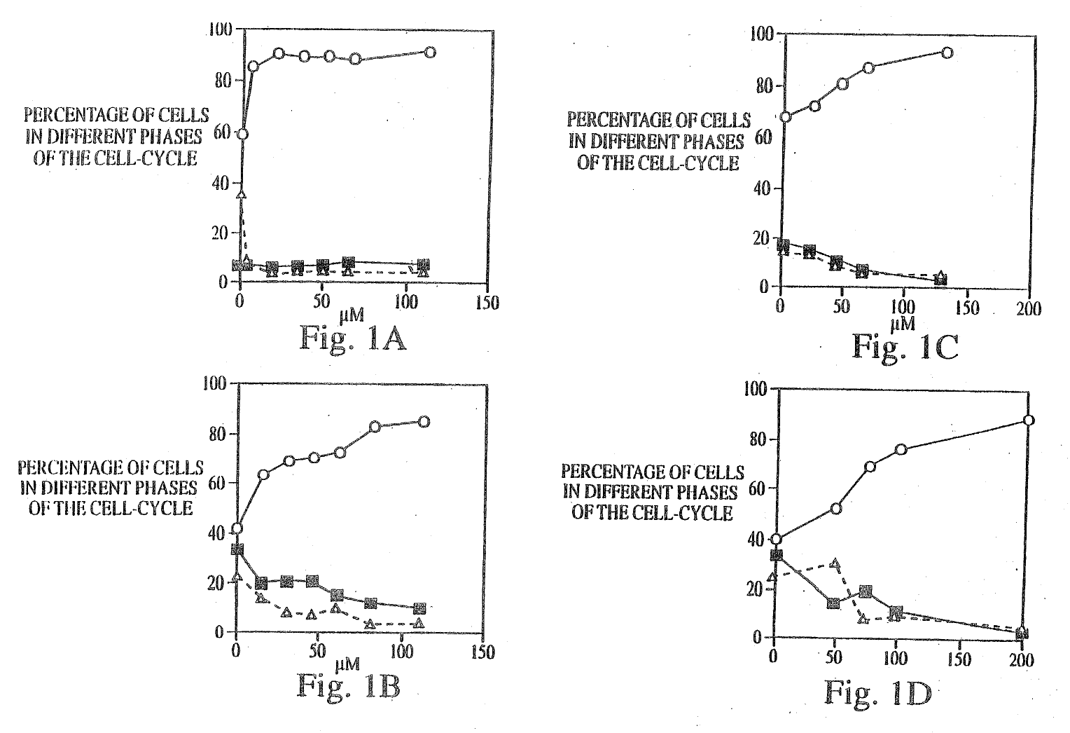

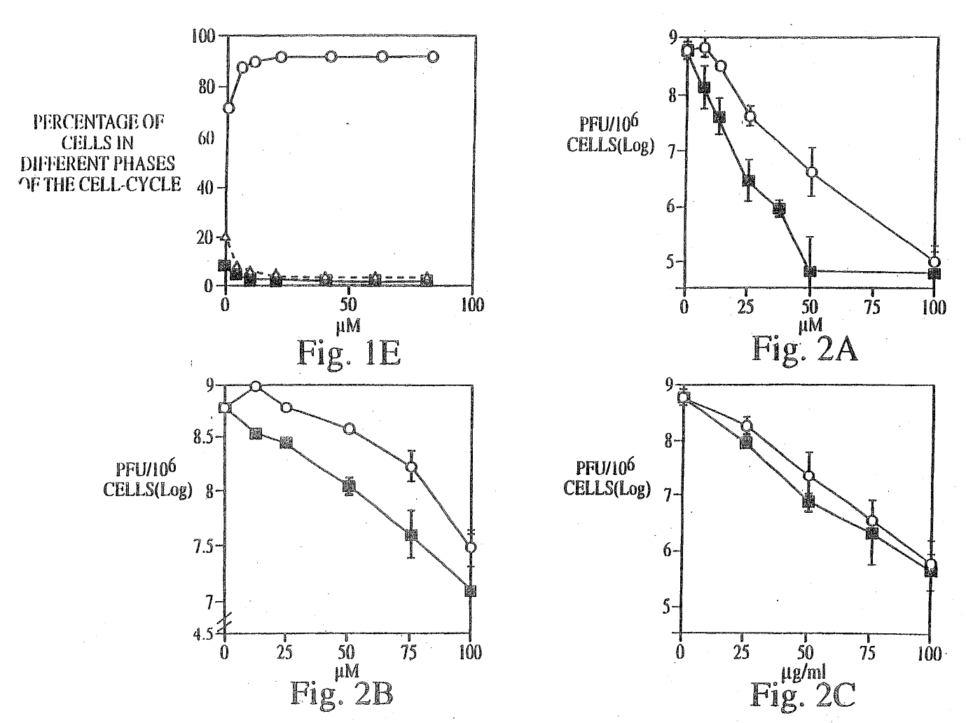

Selection was performed in Vero cells because they tolerate higher concentrations of Rosco and Olo than HEL cells (FIG. 1). In the case of PAA selection, 1 Vero cells were infected with 10³ PFU of KOS stock in the presence of 50 μg/ml of PAA. When viral CPE became evident (about 10 visible plaques in a 35 mm dish), cells were harvested and sonicated, and 100 μl of this stock was used for the second selective passage. From the second passage on, virus was grown in the indicated concentrations of PAA for a total of 11 passages, harvesting the virus in each passage at 4+ CPE. Except for passage 1, in which virus required 4 days to replicate, virus in PAA selection medium was passed every 24 to 72 hours. For Rosco selection, 10⁴ PFU of HSV-1 KOS was used to infect 10⁵ Vero cells in the presence of 50 μM of Rosco. When CPE became evident (about 3 visible plaques in a 35 mm dish), cells were harvested as described above, sonicated, and 500 μl of this stock was used as inoculum for the second selective passage. From the second passage on, virus undergoing selection was harvested at 4+ CPE, or when cells showed signs of drug-induced toxicity, whichever occurred first. If a passage required more than 4 days, medium was changed on the fourth day with medium containing only 75% of the concentration of Rosco used in the first 4 days. Since viral titers had dropped in later passages, the volume of the previous passage stock used as inoculum was increased to 750 to 1000 μl (depending on the titer of each passage).

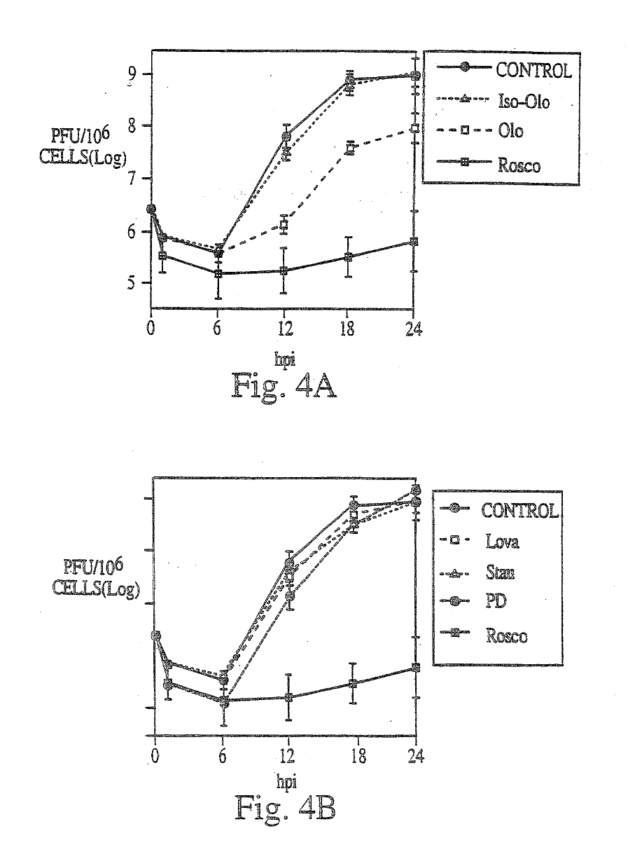

For Olo selection, virus was passaged every 24 hours for the first 8 passages, because since wild type HSV is able to replicate in the presence of Olo (FIG. 4), we were concerned that wild-type virus would outgrow any mutant population during longer passage. Preliminary results indicated that no Olo resistant virus had been selected for in these passages; thus during the final 4 passages, the virus was grown to 4+ CPE, which required 3 or more days.

Probes

Plasmids prp TK, prp8, prp4 (Jordan et al., 1997, J. Virol. 71:6850-6862), p0Hc-Xh and pTRIGAPDH (Ambion, Austin, Tex.) were linearized with HindIII, NcoI, XcmI, NruI and HindIII, respectively. Riboprobes were synthesized using the Riboprobe in vitro transcription system (Promega), following the manufacturer's instructions except that 5 μl of [α³²P]GTP (800 Ci/mmol) was used for label, and no cold GTP was included in the transcription mix. Labeled probes were separated from non-incorporated nucleotides using NucTrap probe purification columns (Stratagene, La Jolla, Calif.).

Viral DNA Replication Assays

9×10⁵ HEL cells were infected with 2.5 PFU per cell of HSV-1 KOS in the presence of 40 μM Rosco, 75 μM Olo, or in the absence of drug. At the indicated times after infection, medium was removed, monolayers were washed with cold PBS, and cells were scraped into 1.0 ml of DNA extraction buffer [0.5% SDS and 50 μg/ml of proteinase K in TEN buffer (10 mM Tris.Cl, 25 mM EDTA, 100 mM NaCl, pH 8.0)]. DNA was extracted as described (Schang et al., 1994, J. Virol.68:8470-8476), and resuspended in TE (10 mM Tris, 1 mM EDTA, pH 7.6) to a final concentration of about 100 ng/ml. Ten μg of DNA from each sample were diluted to 400 μl with TE and alkali-denatured with 40 μl of 3N NaOH at 70° C. for 50 minutes. Afterwards, samples were cooled to room temperature and neutralized with 440 μl of 2M sodium acetate (pH 5.2). Neutralized samples were vacuum slot-blotted to a nylon membrane (GeneScreen, New England Nuclear research products, Boston, Mass.). DNA was UV cross-linked, prehybridized for 1 hour at 75° C. in ExpressHyb solution (Clontech, San Francisco, Calif.) and hybridized with a pool of riboprobes specific for TK and ICP8 (3×10⁶ CPM/ml of each). After one hour of hybridization at 75° C. in ExpressHyb solution, the membrane was rinsed with 4× SSPE (1× SSPE: 0.15 M NaCl, 0.01 M NaH₂PO₄, 0.001 M EDTA, pH 7.4) containing 0.1% SDS, washed twice at room temperature with the same solution for a total of 20 minutes, and once for 15 minutes at 75° C. in 0.2× SSPE, 0.2% SDS. The hybridized membrane was exposed to a PhosphoroImager cassette (Molecular Dynamics, Sunnyvale, Calif.) overnight and stripped in boiling 0.05× SSPE, 1% SDS. After stripping, the membrane was re-exposed, pre-hybridized in ExpressHyb solution for 1 hour at 68° C., and hybridized with 2.5×10⁶ cpm/ml of the GAPDH-specific riboprobe. After hybridizing for 1 hour at 65° C., the membrane was rinsed in 4× SSPE, 0.1% SDS, washed three times for a total of 35 minutes at room temperature in the same solution, once for 5 minutes at 50° C. in 0.1× SSPE, 0.1% SDS and exposed again in the PhosphoroImager cassette. Quantitation of the signal was performed using ImageQuant (Molecular Dynamics, Sunnyvale, Calif.), and the virus-specific signal was standardized to the cellular-specific signal.

RNase Protection Assays

RNase protection assays were performed using the DirectProtect kit following the manufacturer's instructions (Ambion), with minor modifications. 7.5×10⁵ HEL cells were infected with 2.5 PFU per cell of HSV-1 in the presence of 40 μM Rosco, 75 μM Olo, or in the absence of either drug. At the indicated times after infection, medium was removed, monolayers were washed twice with cold PBS, scraped in 150 μl of RNA extraction buffer (DirectProtect, Ambion), and the resulting cell extracts were transferred to Eppendorf tubes. Aliquots (25 μl) of each sample were annealed with 5 to 6×10⁵ cpm of each of the viral-specific probes at 55° C. Another 25 μl aliquot of each sample was annealed with 5.5×10⁵ cpm of the GAPDH specific probe at 37° C. Preliminary experiments had determined that the amount of probe used was saturating at this cell extract to probe ratio. All annealing reactions were performed overnight in a volume of 50 μl. RNase and proteinase digestion were performed according to manufacturer's instructions, and the protected fragments were resolved by electrophoresis in 6% denaturing polyacrylamide gels. Dried gels were exposed and analyzed in a PhosphoroImager system (Molecular Dynamics).

The Results of the experiments presented in the present Example are now described.

The Concentrations of Rosco and Olo that Inhibit Cell-Cycle Progression in HEL and Vero Cells Differs

Different doses of Rosco or Olo have been shown to be required to inhibit cdk activity in different cell types, depending presumably) upon the levels of active cdk in each cell type (Iseki et al., 1997, Surgery 122:187-194; Meijer et al., 1997, Eur. J. Biochem. 243:527-536; Abraham et al., 1995, Biol. of the Cell 83:105-120; Glab et al., 1994 FEBS Letters, 353:207-211; Vesely et al., 1994, Eur. J. Biochem. 224:771-786; Graves et al., 1997, Anal. Biochem. 248:251-257). Because inhibition of cdk activity in vivo results in cell-cycle arrest, we used FACS analysis to determine the concentrations of each drug needed to arrest cell-cycle progression in HEL and Vero cells. Preliminary experiments demonstrated that HEL cells tolerated 75 μM Rosco and 100 μl Olo and that Vero cells tolerated 120 μM Rosco and 200 μM Olo without evidence of toxicity as evaluated by microscopic observation. As shown in FIG. 1A, concentrations of 20 μM or higher of Rosco blocked cell-cycle progression of HEL cells. Cells were blocked primarily in G0/G1 (about 85%) and secondarily in G2/M (about 8%). Although 30 μM Rosco had some effect on cell-cycle progression of Vero cells, concentrations as high as 80 μM were required to block 85% of these cells in G0/G1, about 10% being blocked in G2/M (FIG. 1B). Because Olo is known to be less potent than Rosco (Meijer et al., 1997, Eur. J. Biochem. 243:527-536; Vesely et al., 1994, Eur. J. Biochem. 224:771-786; Iseki et al., 1997, Surgery 122:187-194), it was expected that higher doses of Olo would be required to block cell cycle progression in both cell lines. Indeed, concentrations of Olo below 20 μM had no major effect on HEL cells. Increasing concentrations of Olo between 20 and 65 μM progressively blocked HEL cell-cycle progression more efficiently, with only a minor effect at a higher concentration (FIG. 1C). In contrast, 50 μM of Olo had only a modest effect on Vero cell-cycle progression. However, concentrations of Olo between 50 and 100 μM blocked cell-cycle progression incrementally, whereas little additional inhibition was evident at 200 μM (FIG. 1D). HEL cells treated with Lovastatin (Lova), which blocks cell-cycle progression by indirectly inhibiting transduction of growth-inducing signals across the cytoplasmic membrane, were also examined by FACS analysis. In these tests, Lovastatin (Lova) proved to be the most efficient drug in blocking cell-cycle progression (FIG. 1E). Thus, 5 μM Lova blocked ˜90% HEL cells in G0/G1, as described for this inhibitor in other cell lines (Keyomarsi et al., 1991, Cancer Research 51:3602-3609). Since concentrations of Lova as low as 5 μM had some toxic effect on Vero cells, we did not analyze further the effects of this drug on Vero cells.

These experiments demonstrate that: i) higher concentrations of Rosco and Olo are required to block cell-cycle progression in Vero than in HEL cells; ii) Rosco is more potent than Olo in blocking cell-cycle progression in both cell types, and iii) Lovastatin was the most potent cell-cycle inhibitor of HEL cells.

The Concentrations of Rosco and Olo that Inhibit HSV Replication in HEL and Vero are Similar to Those that Inhibit Cell-Cycle Progression

If cdk activity were required for HSV replication, viral replication in HEL or Vero cells should be inhibited by concentrations of Rosco and Olo that inhibit cdk activity, as measured by inhibition of cell-cycle progression. The effects of different concentrations of Rosco and Olo on single-step HSV-1 replication were analyzed. Viral titers were expressed in PFU/10⁶ cells because this unit allows comparison of titers independent of the number of infected cells and volume of media used to overlay the cultures after inoculation. The age and density of monolayers were approximately the same in all cultures prior to infection.

Similar to the situation for inhibition of cell-cycle progression in uninfected cells, higher doses of both drugs were required to block HSV replication in Vero than in HEL cells (FIG. 2A and FIG. 2B). Thus, 50 μM Rosco was sufficient to inhibit virus replication completely in HEL cells, whereas 100 μM was required to achieve the same effect in Vero cells (FIG. 2A). In agreement with the different concentrations of the two drugs needed to inhibit cdk activity (Meijer et al., 1997, Eur. J. Biochem. 243:527-536; Vesely et al., 1994, Eur. J. Biochem. 224:771-786) and cell-cycle progression (FIG. 1), Olo was less effective than Rosco in inhibiting HSV replication in both cell types. Thus, 50 μM or less of Olo had only modest effects on 24 hour viral titers in Vero cells, whereas those same concentrations were sufficient to reduce viral yields significantly in HEL cells (FIG. 2B). Concentrations of Olo between 50 and 100 μM progressively and more efficiently blocked HSV replication in both Vero and HEL cells. Because the concentrations of Rosco and Olo needed to inhibit cell-cycle progression are similar to those needed to inhibit viral replication in HEL and Vero cells, and because these concentrations are proportional to the concentrations of both drugs required to inhibit cdk activity in vitro (Meijer et al., 1997, Eur. J. Biochem. 243:527-536; Vesely et al., 1994, Eur. J. Biochem. 224:771-786), it is postulated that the inhibition of HSV replication by these drugs is through effects on cellular cdk(s) in virus-infected cells. In contrast to Rosco and Olo, phosphonoacetic acid (PAA), a drug that directly inhibits the activity of an essential virally-encoded function (DNA polymerase), is known to inhibit HSV replication in different cell-types at approximately the same concentration (Jofre et al., 1977, J. Virol. 23:833-836; Honess et al., 1977, J. Virol. 21:584-600; Hay et al., 1976, J. Gen. Virol. 1:145-148; Becker et al., 1977, Antimicrobial Agents & Chemotherapy 11:919-922; Overby et al., 1974, Antimicrobial Agents and Chemotherapy 6:360-365). Indeed, unlike Rosco and Olo, PAA inhibited viral replication with similar potency in HEL and Vero cells, although it was slightly more potent in HEL than in Vero cells at all concentrations tested (FIG. 2C).

These experiments demonstrate that: i) as for inhibition of cell-cycle progression, higher concentrations of Rosco and Olo are required to block HSV replication in Vero than in HEL cells, ii) Rosco is more potent than Olo in blocking HSV replication; and iii) Rosco inhibits HSV replication as efficiently as PAA.

Inhibition of HSV Replication by Rosco is Not a Consequence of Cytotoxicity

Although Rosco and Olo did not induce detectable cytopathology as determined by microscopic evaluation at the concentrations used to block HSV replication, it remained a possibility that more subtle toxic effects of these drugs may have compromised the ability of cells to support HSV replication. To determine whether this was the case, a Rosco reversal experiment was performed. Rosco was selected for these experiments because it inhibits HSV replication more efficiently than Olo. Therefore, any increase in viral replication after removing the drug would be more obvious. HEL and Vero cells were therefore infected with 2.5 PFU per cell of HSV in the presence of 40 (HEL) or 100 μM (Vero) Rosco, and medium was replaced 24 hours later with fresh medium containing no drug. At selected times before and after the change of medium, cells were harvested and the amount of infectious virus was determined by standard plaque assay (FIG. 3). Under these circumstances, the 24 hour yield was reduced by 3 (HEL) or 4 (Vero) orders of magnitude as demonstrated previously for those concentrations of Rosco (FIG. 2), yet resumption of HSV replication was evident in HEL cells 6 hours after release from the Rosco-induced block (FIG. 3A). Twenty-four hours after release, viral titers approached (in HEL cells) or reached (in Vero cells) those attained in untreated cultures (FIG. 3B). In contrast, when medium was replaced with fresh medium containing Rosco, viral titers did not increase during 24 hours after release in either cell type (FIG. 3). Cytotoxicity was evident in cells infected with 2.5 PFU per cell of HSV-1 and treated with Rosco for more than 24 hours. Since this toxicity was not observed in Rosco-treated uninfected cells, we conclude that the combined effect of the drug and the infection is the most likely cause of toxicity. Similar cytotoxicity has been observed during HSV infection in the presence of PAA (Honess et al., 1977, J. Virol. 21:584-600. Thus, Rosco-induced inhibition of HSV replication was not mediated by irreversible drug-induced toxicity in either HEL or Vero cells.

Inhibition of HSV Replication is Specific for cdk Inhibitors

Although the specificities of Rosco and Olo have been evaluated so extensively (Meijer et al., 1997, Eur. J. Biochem. 243:527-536; Vesely et al., 1994, Eur. J. Biochem. 224:771-786) that these drugs are currently used to confirm involvement of cdks in biological processes (Kwon et al., 1997, Proc. Natl. Acad. Sci. USA 94:2168-2173; Wieprecht et al.,1996, J. Biol. Chem. 271:9955-9961; Bresnahan et al., 1997, Virology 231:239-247), it remained a theoretical possibility that the findings presented in FIGS. 2 and 3 are a consequence of a block in other cellular protein S/T kinase(s), or of direct inhibition of a virus-encoded function.

The following series of experiments were performed to assess these possibilities.

First, HSV replication in HEL cells was measured in the presence and absence of Olo, Rosco, IsoOlomoucine (a structural isomer of Olo that does not inhibit cdk activity) (Vesely et al., 1994, Eur. J. Biochem. 224:771-786; Wieprecht et al.,1996, J. Biol. Chem. 271:9955-9961), Lova (a cell cycle inhibitor that does not block cdk activity) (Keyomarsi et al., 1991, Cancer Research 51:3602-3609), Staurosporine (a broad-spectrum protein S/T kinase inhibitor) (Rüeg et al., 1989, Trends in Pharmacol. Sci. 10:218-220) or PD98059 (a specific inhibitor of erk-1 and erk-2) (Dudley et al., 1995, Proc. Natl. Acad. Sci. USA 92:7686-7689). HEL cells were used in these tests because HSV replication was inhibited in these cells with lower concentrations of Rosco or Olo. Thus, any inhibitory effect of the other drugs tested on HSV replication should be more easily detected in HEL than in Vero cells. Since the stocks of all drugs except Lova were prepared in DMSO, preliminary control experiments were conducted to determine that a 1:500 dilution of DMSO in medium had no inhibitory effect on HSV replication.

In “single-cycle” growth experiments, Olo inhibited HSV replication by nearly two orders of magnitude through 12 hours pi, but it was less inhibitory thereafter (FIG. 4A), consistent with other recognized biological effects of this drug (Vesely et al., 1994, Eur. J. Biochem. 224:771-786). Rosco, on the other hand, blocked HSV replication almost completely throughout the 24 hour test period (FIG. 4). A structural isomer of Olo which does not inhibit cdk activity, Iso-Olo (Vesely et al., 1994, Eur. J. Biochem. 224:771-786), had no detectable effect on viral replication, suggesting that the observed inhibition of HSV replication by Olo was indeed mediated by inhibition of cellular cdks (FIG. 4A).

Because HSV replicates in growth-arrested cells, it was likely that Lova, which like Rosco and Olo arrests cells in G1 (but by a different mechanism), should not inhibit HSV replication. As anticipated, 20 μM Lova, which arrests about 90% of HEL cells in G0/G1 (FIG. 1E), failed to block HSV replication, confirming (Shadan et al., 1994, J. Virol. 68:4785-4796) that simple cell-cycle arrest in G0/G1 is not sufficient to block HSV replication (FIG. 4B).

Because Rosco and Olo inhibit erk-1 and 2 (although about 20- and about 10-fold less efficiently, respectively than they inhibit cdks) (Meijer et al., 1997, Eur. J. Biochem. 43:527-536; Vesely et al., 1994, Eur. J. Biochem. 224:771-786), the question of whether these kinases were required for HSV replication was asked. As shown in FIG. 4B, PD98059, a drug that specifically inhibits erk-1 and 2 but not cdks (Dudley et al., 1995, Proc. Natl. Acad. Sci. USA92:7686-7689), did not inhibit HSV replication at a concentration (70 μM) above concentrations that inhibit erk1 and 2 in vivo (Dudley et al., 1995, Proc. Natl. Acad. Sci. USA 92:7686-7689). Similar results were been observed in PC12 cells. Obvious cytotoxic effects were observed by microscopic evaluation when uninfected HEL cells were treated with concentrations of this drug higher than 70 μM.

Although Rosco and Olo are highly cdk-specific drugs (Meijer et al., 1997, Eur. J. Biochem. 243:527-536; Vesely et al., 1994, Eur. J. Biochem. 224:771-786), a broad-spectrum protein S/T kinase specific inhibitor was also used to further test the initial hypothesis that the block in HSV replication was mediated by the inhibition of cdks, and not by a non-specific inhibition of other protein S/T kinases. Staurosporine, a widely used broad-spectrum protein S/T kinase inhibitor, did not block HSV replication significantly at a concentration at which it inhibits protein S/T kinases, 5 ng/ml (111 nM) (Rüeg et al., 1989, Trends in Pharmacological Sciences 10:218-220; Omura et al., 1995, The J. Antibiotics 48:535-548) (FIG. 4B). Similar results have been observed with another broad-spectrum protein S/T kinase inhibitor, K5720, in PC12 cells (Jordan et al., 1998, Journal of Virology, submitted). Higher concentrations of Staurosporine, which may inhibit cdk-2 in vivo (i.e., 100 to 200 ng/ml) Crissman et al., 1991, Proc. Natl. Acad. Sci. USA 88:7580-7584; Omura et al., 1995, The J. Antibiotics 48:535-548), could not be tested because they are toxic for both HEL and Vero cells.

Thus, experiments using inhibitors demonstrated that HSV replication was inhibited only by inhibitors of cdks, and not by an isomer of a cdk inhibitor which does not inhibit cdk activity, a cell cycle inhibitor that arrests cells by a mechanism not directly involving cdks, or a broad-spectrum inhibitor of protein S/T kinases.

Rosco and Olo do not Target an HSV-1 Encoded Function: Failure to Isolate Drug-Resistant Mutants

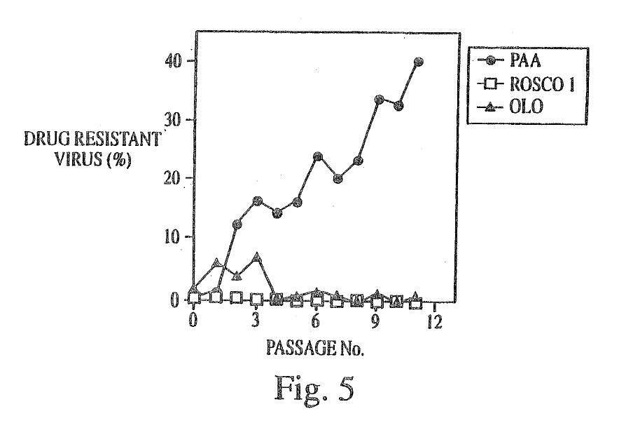

Characteristically, drugs that block HSV replication by direct inhibition of (a) viral function(s) can be used to select for genetic variants that are resistant to the drug. Therefore, if Rosco or Olo directly inhibit a virus-encoded function, it should be possible to isolate spontaneous drug-resistant mutant(s) by serial passage of virus in the presence of the drugs. Consequently, standard procedures for isolating drug-resistant mutants were employed in an effort to select Rosco and Olo-resistant HSV-1 mutants. Briefly, HSV-1 was passed several times in the presence of the “selection” drugs, starting at sub-inhibitory concentrations and increasing the concentrations in successive passages. Except for the first passage (when virus was harvested after a few plaques were visible), virus was harvested when cytopathic effects were generalized (4+ CPE), or when drug-induced cellular toxicity was evident, whichever occurred first. As a positive control, a parallel selection in PAA was conducted. PAA specifically targets the virus encoded DNA polymerase (Overby et al., 1974, Antimicrobial Agents and Chemotherapy 6:360-365). Preliminary tests demonstrated that the plaque purified HSV-1 KOS stock contained about 1 PAA-resistant infectious virus per 10⁴ PFU, in agreement with previous findings (Jofre et al., 1977, J. Virol. 23:833-836; Becker et al., 1977, Antimicrobial Agents & Chemotherapy 11:919-922). Therefore, 10³ PFU of KOS stock was used to infect 10⁵ Vero cells in the presence of 50 μg/ml of PAA in the first passage, and virus was further passaged in increasing concentrations of PAA for a total of 11 passages (FIG. 5). The concentration of PAA used in the eleventh passage was 500 μg/ml, 5 times the concentration necessary to inhibit replication of unselected HSV stocks. The 11 passages were completed in 27 selection days. For selection in Rosco or Olo, the inocula for the first passages consisted of 10⁴ PFU of HSV-1 KOS (the same stock used for PAA selection), because no Rosco or Olo-resistant PFU were identified in preliminary experiments. For Rosco selection, a procedure that paralleled that used for PAA selection was followed as closely as possible. However, in the third passage, when the concentration of Rosco was increased from 50 to 75 μM, viral replication was severely impaired, such that the concentration of drug was reduced in the following passage. Consequently, the “Rosco selected” stock was split into two parts after passage 6. One half was passaged continuously in low concentrations of Rosco (30 to 50 μM), while the intention was to select the other half in increasing concentrations of the drug, Rosco 1b and Rosco 1, respectively). As had occurred previously, when the concentration of Rosco was increased (from 50 to 75 μM) in passage 7, viral titers dropped markedly such that 50 μM of this drug was used for passage 8, and 30 μM was used for the following two passages. Although the initial basis for splitting the stock undergoing selection proved to be untenable, the experiment was continued, in that, both stocks were under selection in an effort to increase the probability of identifying Rosco resistant variants. Eleven passages in Rosco required 34 selection days; 10 passages in “low” concentrations of Rosco (all passages after passage 6 were in 30 to 50 μM) required 30 selection days. The concentration of Rosco in the final passage for both selection lines was 50 μM, the same concentration that inhibited replication of unselected HSV-1 in the first passage. For Olo “selection”, the concentration of the drug was increased from 75 to 150 μM from passage 1 to 4, but could not be increased further because Olo is cytotoxic for HSV-1 infected Vero cells when used at 200 μM for more than 24 hours. Eleven passages in Olo “selection” required 20 selection days. Rosco may have blocked viral replication too efficiently to permit selection of resistant mutants. Reasoning that an HSV stock “preselected” in Olo might be further selected in Rosco, 10⁶ PFU from the ninth passage in Olo were used as inoculum for 11 successive passages in Rosco (for a total of 20 passages), following the same protocol described above for “Rosco selection” of HSV-1 stock, including the branching in “high” (Rosco 1) and “low” (Rosco 1b) concentrations of drugs after passage 6.

When all “selection lines” had undergone 11 passages, 1000 PFU of each passage selected in PAA, Olo, or Rosco were used to infect 10⁵ Vero cells, in the presence or absence of 100 μg/ml PAA, 150 μM Olo or 100 μM Rosco, respectively. Viral titers were too low to allow infection with 1000 PFU in many Rosco passages (selection series 1: passages 1, 3, 4, 5, 6, 7 and 8; selection series 1b: passages 7 and 8; selection series 9: passages 7 and 8). For these passages, the highest inoculum possible was used, which varied from about 50 to about 500 PFU. Twenty four hours after infection, virus was harvested, the percentage of drug-resistant virus in the total yield was calculated using the formula: “% resistance”=100×(PFU in selection drug)/(PFU in drug-free medium), and this percentage was plotted as a function of passage number (FIG. 5). Standard plaque assays could not be used for this purpose because at the concentrations of Rosco required to fully inhibit HSV replication, Vero cells overlaid with methyl cellulose do not form an homogeneous monolayer and are thus unsuitable for plaque counting.

In agreement with previously published results (Honess et al., 1977, J. Virol. 21:584-600), some PAA resistant virus was detected as early as passage 2 (FIG. 5). By passage 11, titers in the presence of PAA were approximately 50% of the titers in absence of the drug. Unlike selection in PAA, it was not possible to detect Rosco or Olo-resistant mutants of HSV after 11 passages in drug-containing media (FIG. 5). A complete comparison of the four Rosco selection was conducted. No resistant virus was selected in these cultures and, furthermore, no trend towards development of Rosco-resistance was observed either.

These experiments established that Rosco and Olo blocked HSV replication by inhibition of cellular function(s), or that the viral functions targeted by these drugs are either numerous or so essential for viral replication that mutation(s) in the genes encoding these functions are lethal.

The Accumulation of Viral Immediate-Early and Early mRNA is Reduced in the Presence of Rosco and Olo

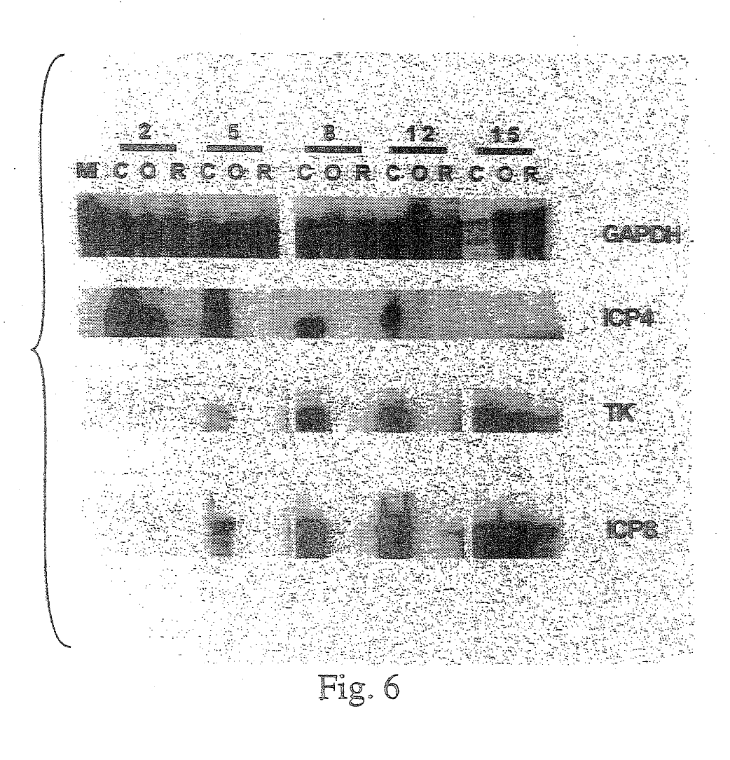

The results presented above could be interpreted to mean that viral DNA replication is a direct target of inhibition by Rosco and Olo. Alternatively (but not mutually exclusive), viral DNA replication could be inhibited indirectly by these drugs through inhibition of viral immediate-early or early gene expression. Expression of selected viral immediate-early and early genes was therefore analyzed at the level of mRNA accumulation in the presence and absence of Rosco or Olo. For this purpose, HEL cells were infected with 2.5 PFU per cell of HSV-1 in the presence and absence of 75 μM Olo or 40 μM Rosco, and total infected cell RNA was extracted at the indicated times post infection (FIG. 6). Levels of an mRNA of a cellular house-keeping gene, GAPDH, decreased at late times after infection with wild type HSV-1 in the absence of either drug. Levels of GAPDH did not decrease in cells infected in the presence of Rosco or Olo, consistent with the inhibition of viral replication by these drugs. In contrast, expression of immediate-early ICP4 mRNA was greatly impaired by Rosco and Olo as early as 2 hours pi (FIG. 6). Accumulation of TK and ICP8 mRNAs was also reduced by Rosco or Olo at later times (5, 8 and 12 hours pi). At 15 hours pi, however, levels of TK and ICP8 RNAs increased significantly in the Olo-treated cells, consistent with the increased levels of viral replication and viral DNA synthesis that occur in the presence of Olo as noted above. A slight increase in the levels of ICP8 and TK RNAs was also observed in the Rosco-treated samples. Moreover, no decrease in the half-life of viral mRNAs was apparent under these conditions. Therefore, the low levels of viral transcripts in the presence of Rosco or Olo are a consequence of a block in viral transcription.

It must be emphasized that the RNase protection assay was optimized to require minimal handling of the samples, rather than to achieve maximal sensitivity. Thus, the absence of signal in a given lane does not necessarily imply the absence of the measured mRNA, but inability to measure low levels of mRNA. Because levels of selected immediate-early and early transcripts are reduced by Rosco and Olo, yet functional immediate-early and early proteins are required for viral DNA replication, it was not possible to discern whether these drugs block viral DNA replication directly through inhibition of a (cellular) protein kinase essential for this process, or indirectly through effects on immediate-early and early gene expression. Moreover, the reduced levels of early transcripts detected can themselves be a secondary effect of the reduced levels of immediate-early transcripts and, consequently, proteins.

### EXAMPLE 2

### Cellular Cyclin-Dependent Kinases (cdks) are Required for Transcription of Herpes Simplex Virus (HSV) Immediate-Early and Early Genes and for Viral DNA Replication

The information presented in this Example is summarized as follows.

Although HSV replicates in both cycling and non-cycling cells, including terminally-differentiated neurons, the data presented in Example 1 establish that HSV replication requires the activities of cellular cyclin-dependent kinases (cdks). Those data also established that transcription of HSV immediate-early genes is impaired in the presence of two highly specific cdk inhibitors, Olo and Rosco. Based on the inability to isolate HSV mutants resistant to these cdk inhibitors, it was postulated that cdks may be required for more than one step in the HSV replication cycle. In the experiments presented herein, this hypothesis was tested by measuring the efficiency of viral replication, expression of selected viral immediate-early (ICP0 and ICP4), early (ICP8 and TK) and late (gC) transcripts, and viral DNA replication during infections in which Rosco was added after immediate-early and/or early gene products had already been expressed. Specifically, Rosco was added at selected times post-infection, or after release from cycloheximide (CHX) block. Rosco inhibited HSV replication, immediate-early and early gene transcription, as well as viral DNA replication, efficiently when added at 1, 2, or 6 hours pi, or after release from a CHX block. Transcription of a representative late gene was inhibited by Rosco under all conditions examined. These data establish that cellular cdks are required for transcription of HSV immediate-early and early genes The Materials and Methods used in the present Example are now described.

Cells, Virus, Plasmids and Drugs

Methods used for the propagation and maintenance of HEL and Vero cells are described in Example 1. A plaque purified low passage (p9) stock of HSV-1 strain KOS was used throughout these studies and prepared as described (Schang et al., 1998, Journal of Virology 72 (7):5626-5637). The construction of plasmids prpTK, prp8, prp4, and prpgc has been described in (Jordan et al., 1997, Journal of Virology 71:6850-6862).

Rosco and PAA were prepared and diluted as described in Example 1. Cycloheximide (CHX) was prepared in PBS as a stock at concentration of 20 mg/ml. Stock CHX solution was further diluted to the indicated working concentration in D-MEM containing 10% fetal bovine serum (FBS). Final concentrations of drugs were 100 μM Rosco, and 50 μg/ml CHX in all experiments.

Infections

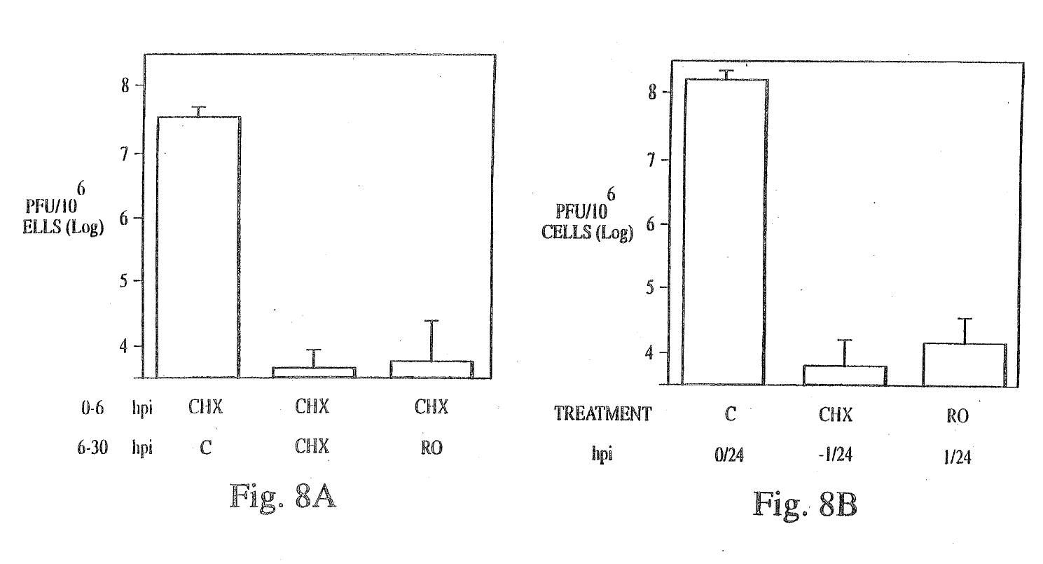

Vero cells (2×10⁵ cells) were infected with 2.5 to 3.0 PFU per cell of virus diluted in serum-free medium. After adsorption for 1 hour at 37° C., the viral inoculum was removed, monolayers were washed twice with cold PBS, and standard medium or medium containing drugs was added as indicated. When indicated, medium overlaying infected cells was replaced with fresh drug-containing or control medium. Infected cells were scraped into the medium at the indicated times after infection (where T=0 is the time of addition of inoculum), and the entire infected cell suspension was transferred to a 5.0 ml tube and frozen at −70° C. After thawing, cells were sonicated for 45 seconds, and infectious virus was titrated by standard plaque assay. For experiments in which the time of addition of Rosco was varied (experiments presented in FIGS. 8 to 11), drug-free medium was removed from infected cells at 2 or 6 hours pi and was replaced with two volumes of drug-containing medium. For the drug-replacement and drug-release experiments, drug-containing medium was removed from infected monolayers at the indicated times post infection. Infected cells were then washed twice with PBS containing the same concentration of drug to be added to the respective well after the washes. After washing, two volumes of drug-free medium, or medium containing CHX, or Rosco, were added to each monolayer. Two volumes of medium were used in order to dilute any residual drug remaining on the monolayers after the washes.

Probes

Plasmids prpgC, prpTK, prp8, prp⁴, p0Hc-Xh and pTRIGAPDH, are described in Example 1. The plasmids were linearized with BsgI, HindIII, NcoI, XcmI, NruI, and HindIII, respectively. Riboprobes were synthesized using the Riboprobe in vitro transcription system (Promega, Madison Wis.), following the manufacturer's instructions except that 5 μl of α³²P]GTP (800 Ci/mmol) was used for label, and no cold GTP was included in the transcription mix. Labeled probes were separated from non-incorporated nucleotides using NucTrap probe purification columns (Stratagene, La Jolla, Calif.).

RNase Protection Assays

RNase protection assays were performed using the DirectProtect kit (Ambion) as described in Example 1, with minor modifications. Briefly, 4 to 4.5×10⁶ Vero cells were infected with 2.5 to 3.0 PFU per cell of HSV-1 in the presence of the indicated drug or in control medium. Medium was replaced as indicated. At the indicated times after infection, medium was removed, monolayers were washed twice with cold PBS, scraped into 600 μl of RNA extraction buffer (DirectProtect, Ambion), and the resulting cell extracts were transferred to Eppendorf tubes. Aliquots (45 μl) of each sample were annealed with 5 to 6×10⁵ cpm of each of the viral-specific probes at 55° C. Another 45 μl aliquot of each sample was annealed with 5.5×10⁵ cpm of the GAPDH specific probe at 37° C. Preliminary experiments had determined that the amount of probe used was saturating at this ratio of cell extract to probe. All annealing reactions were performed overnight in a volume of 50 μl. RNase and proteinase digestions were performed according to manufacturer's instructions, and the protected fragments were resolved by electrophoresis in 6% denaturing polyacrylamide gels. Dried gels were exposed and analyzed using a Storm PhosphoroImager system (Molecular Dynamics, Sunnyvale, Calif.).

The Results of the experiments presented in this Example are now described.

Rosco Inhibits HSV Transcription when Added 1 2, or 6 Hours pi

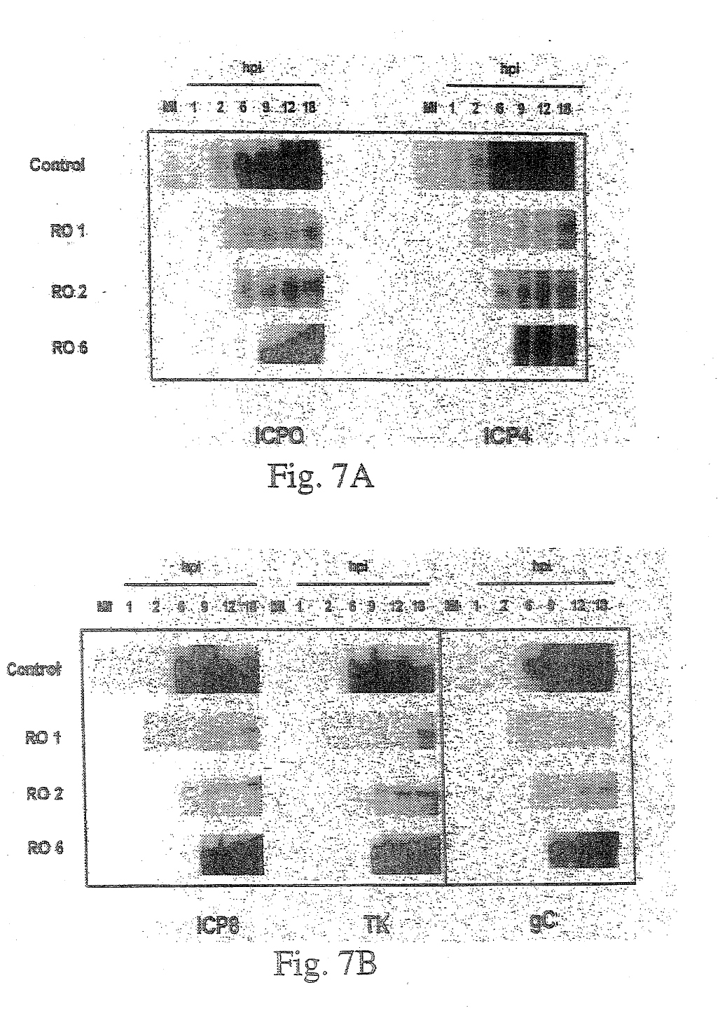

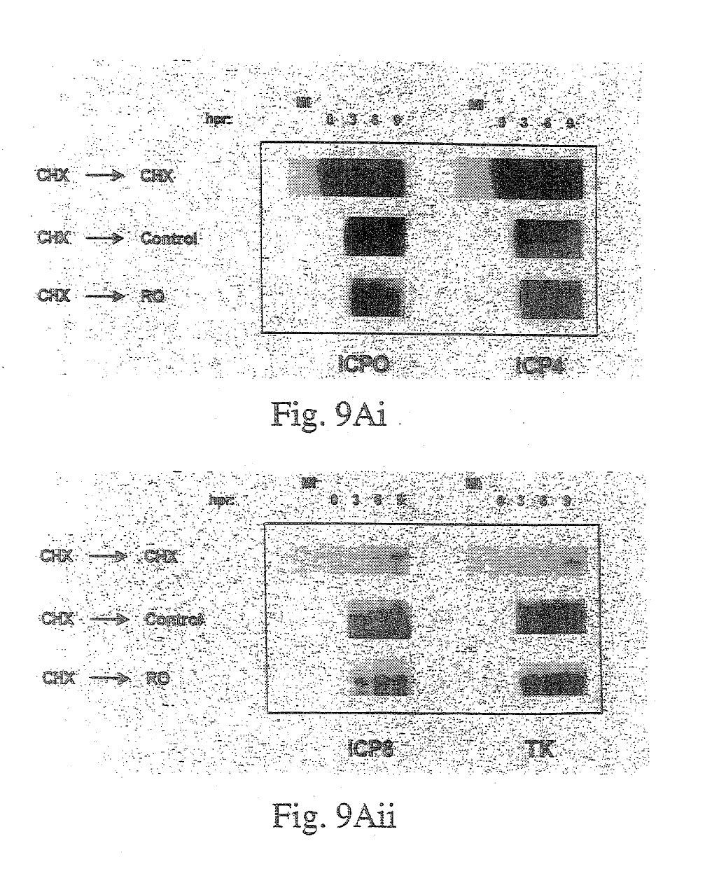

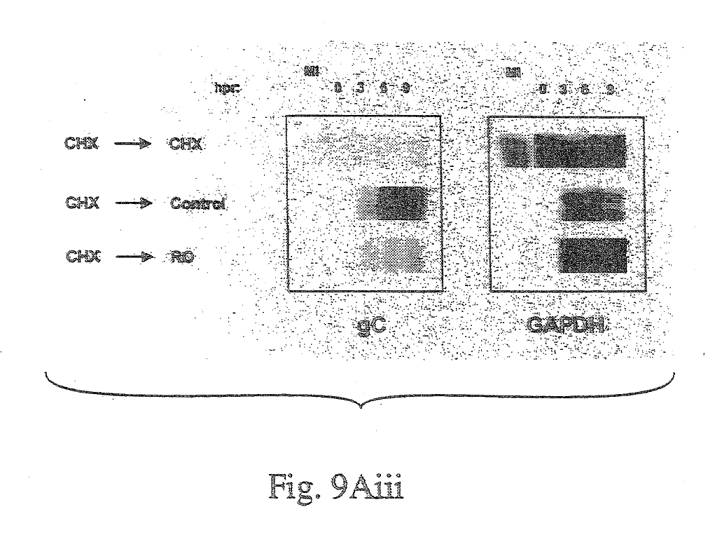

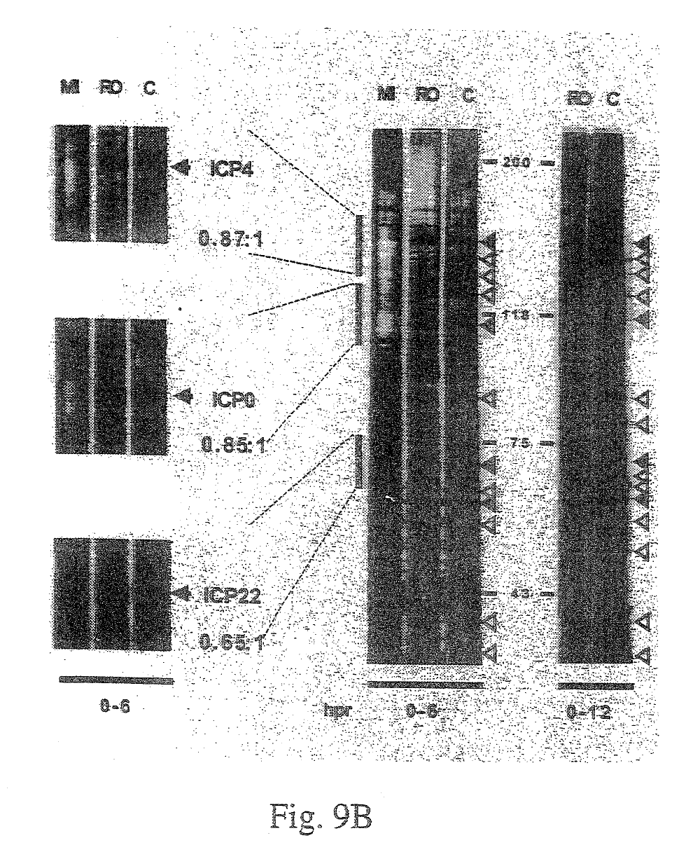

Experiments were next conducted to determine the steps in the HSV replication cycle which were inhibited when Rosco was added at 1, 2, or 6 hours pi. For this purpose, Vero cells were infected for 1 hour, washed and overlaid with drug-free (control) or Rosco-containing medium, and media were changed at the indicated times post infection as described above. At 1, 2, 6, 9, 12 and 18 hours pi, cells were harvested and total infected cell RNA was extracted. Levels of two immediate-early (ICP0 and ICP4), two early (ICP8 and TK) and one late (gC) viral transcript were measured by RNase protection assays as described in Example 1. Equal loading was monitored by measuring the levels of a housekeeping transcript, GAPDH. As previously observed in the experiments conducted in Example 1, when added to Vero cells, Rosco efficiently inhibited the accumulation of HSV immediate-early and early transcripts in Vero cells when added immediately after adsorption (FIG. 7A, RO 1, ICP0, ICP4, ICP8, and TK). Not surprisingly, levels of a late transcript, whose expression is dependent on immediate early and early gene functions, were also very low under these conditions (FIG. 7A, RO 1, gC). Slightly higher levels of all of the transcripts examined were observed when Rosco was added at 2 hours pi (1 hour after adsorption) (FIG. 9, RO 2, all panels). When Rosco was added at 6 hours pi, the levels of early transcripts did not increase significantly with respect to the level attained immediately before the addition of the drug (FIG. 7A, compare 6 hours pi, control with 18 hours pi, RO 6, panels TK and ICP8), suggesting that no new viral early transcripts were synthesized after addition of the drug. The levels of immediate-early and late transcripts continued to increase after the addition of Rosco at 6 hours pi, although the rate of accumulation of these transcripts in the presence of Rosco added at 6 hours pi was significantly lower than in the control untreated infections (FIG. 7A, compare 6 hours pi, control with 18 hours pi, RO 6, panels ICP0, ICP4, and gC).

Rosco Inhibits HSV Replication when Added After 6 Hours of Infection in the Presence of CHX

It was technically impossible to add Rosco to infected cells at a time when all viral proteins required for viral DNA replication were expressed, but initiation of viral DNA replication had not yet occurred. Moreover, because of the overlap between the times of expression of HSV immediate-early and early gene products, it was also impossible to evaluate the direct effects of Rosco on early gene expression by adding the drug at different times after infection. An alternative approach was therefore employed. The effects of Rosco on specific stages of the HSV replication cycle were addressed by blocking the progress of infection using compounds known to perform this function, and then releasing the block in virus replication in the presence or absence of Rosco.

The effects of addition of Rosco on virus replication when high levels of IE transcripts had already been expressed was examined. For this purpose, a CHX-release experiment was performed and the results are presented in FIG. 8. Vero cells were pre-treated with CHX for 1 hour, infected with 3 PFU/cell of HSV-1 and overlaid with medium containing 50 μg/ml of CHX. The concentration of CHX used in these and subsequent reversal experiments was minimized to permit efficient reversal. Six hours later, medium was removed from the cells, the monolayers were washed twice with PBS and were overlaid with fresh medium containing no drug (Control), 50 μg/ml CHX, or 100 μM Rosco. The PBS used for the washed contained the same drugs as the media added after the washes. Twenty four hours after the change of medium, the cells were harvested and the amount of infectious virus present was measured in standard plaque assays.

The results presented in FIG. 8A demonstrate that Rosco inhibited HSV-1 replication efficiently when added after removal of CHX at 6 hours pi (CHX/RO). Indeed, inhibition of HSV-1 replication by Rosco under these conditions was almost as efficient as when CHX itself was added back after removal of CHX at 6 hours pi (CHX/CHX). For comparison, cells in three dishes were infected for 24 hours without change of medium. The infected cells in one dish were incubated for 24 hours in drug-free medium (C); the infected cells in a second dish were incubated in the presence of CHX from one hour before infection through 24 hours pi (CHX); and the infected cells in a third dish were incubated in the presence of Rosco from 1 to 24 hours pi (RO) FIG. 8B). Comparison of FIGS. 8A and 8B demonstrates that when Rosco was added after removal of CHX at 6 hours pi, HSV-1 replication was inhibited nearly as efficiently as when Rosco was added immediately after infection.

Rosco Inhibits HSV Transcription when Added 6 Hours After Infection in the Presence of CHX

To determine whether Rosco was capable of inhibiting accumulation of HSV early transcripts when immediate-early gene products were already expressed, the levels of representative viral immediate-early, early, and late transcripts at specific times after a release from a CHX block were measured. After 6 hours in CHX-containing medium, infected cells were either maintained in CHX or switched to Rosco, PAA, or control medium. Vero cells were infected, blocked at T=0 (cells were pre-treated with CHX from 1 hour before infection) and released as described above. Before (0) and 3, 6, and 9 hours after the change of medium (hours post-release, hpr), cells were harvested and RNA was extracted and quantitated as described herein in Example 1. The change to Rosco after 6 hours of incubation in CHX resulted in a significant reduction in the further accumulation of immediate early transcripts (FIG. 9, panels ICP0 and ICP4). Levels of early transcripts were also reduced in Rosco-containing medium, compared to PAA-containing or Control medium under these conditions (FIG. 9, panels ICP8 and TK). Not surprisingly then, levels of a late transcript were significantly lower in CHX-blocked infected cells released in the presence of Rosco, compared to the infected cells released in the presence of control or PAA-containing medium (FIG. 9, panel gC).

As is known in the art, CHX is a general inhibitor of translation. Thus, during infections performed in the presence of this drug, IE transcripts accumulate, but are not translated. Consequently, E promoters are not activated, E transcripts and proteins are not expressed, DNA replication does not occur and infectious virus is not produced. However, CHX inhibition is reversible, such that, when the drug is removed, IE proteins are translated from accumulated transcripts and the replication cycle of the virus resumes. Thus, if cdks are required for HSV replication functions which occur after IE transcription accumulation, Rosco should inhibit events which occur after IE transcription, including transcription and translation of E and L genes and virus replication, when added following reversal of a CHX block.

Although the cdks known to be inhibited by Rosco have not been reported to be required for translation, this remained a theoretical possibility. Thus, a metabolic labeling experiment to determine whether the E transcripts detected in the previous set of data were indeed translated into proteins were performed. Vero cells were treated with CHX for 1 hour. The cells were then infected with 6 PFU/cell of HSV-1 and were maintained in the presence of CHX for 6 additional hours. At 6 hours pi, CHX-containing medium was removed, and the cells were washed with PBS containing either no drug, or containing 100 μM Rosco. After washing, medium containing ³⁵S methionine and 100 μM Rosco (RO) or no drug (Control) was added to the monolayers. At 6 and 12 hours after release from the CHX block in the presence of label and drug, the cells were harvested and the labeled proteins were resolved by SDS-PAGE. For comparison, mock-infected cells (MI) were blocked with CHX for 6 hours and released in the presence of label-containing, drug-free medium. In preliminary experiments, it had been determined that Rosco had no visible effect on cellular proteins synthesis in uninfected cells after release from a 6 hour CHX block. The results of this experiment are shown in FIG. 9B.

Six hours after release into Rosco-containing medium, the majority of the labeled proteins comigrated with cellular proteins in the gel (FIG. 9B, compare MI and RO at 1-6 hours pi). However, at least four labeled bands derived from infected cells which were released into Rosco-containing medium comigrated with labeled bands derived from infected cells which were released into drug-free medium (FIG. 9B, compare RO and C, solid arrowheads). Based on the molecular weights and migration patterns of these bands, these four proteins were identified as the E proteins ICP0, ICP4, ICP22 and ICP27. Notably, the levels of these proteins were similar in infected cells released into Rosco-containing or drug-free medium. Thus, the levels of ICP0 and ICP4 synthesized in the presence of Rosco were about 85% of the levels of these proteins synthesized in drug-free medium, as measured by Phosphorlmager analysis. The levels of one IE protein, ICP22, were slightly reduced in the presence of Rosco (about 65% of “no-drug” levels in this experiment. Although the half-lives of the IE proteins in the presence of Rosco were not assessed, any change in the half-life would be physiologically irrelevant as it would not affect the total amount of accumulated protein in the first 6 hours after release from the CHX block.

In infected cells which were released into drug-free medium, several E and L, as well as IE proteins were detected at 6 hours after release (FIG. 9B, open arrow heads), and the levels of most of these proteins increased during the first 12 hours after release (compare C, 0-6 hpr and 0-12 hrp in FIG. 9B). In contrast, in the infections released in the presence of Rosco, the levels of IE proteins did not increase after 6 hours post release (FIG. 9B, compare RO at 0-6 and 0-12 hpr). In both control and Rosco-treated cultures, the levels of IE proteins were lower when cells were labeled for 12 hours after release than when they were labeled for 6 hours. Therefore, as expected, synthesis of IE proteins was not maintained indefinitely after release. Moreover, the enhanced decrease in levels of IE protein synthesis at later times after release of the CHX block in the presence of Rosco (FIG. 9B, open arrowheads, compare RO and C for 0-6 and 0-12 hpr) correlated with the previously observed decrease in the steady state levels of IE transcripts. No E or L proteins were detected in cells released from the 6 hour CHX block into Rosco-containing medium (FIG. 9B, RO at 0-6 and 0-12 hpr), consistent with the low levels of E and L transcripts that accumulated under these conditions.

In summary, although the levels of IE protein synthesized in the presence of Rosco after a 6 hour CHX block were not significantly lower than in infected cells released into control medium, E transcript accumulation was significantly impaired by Rosco under these conditions. It is therefore concluded that Rosco inhibits transcription of E genes in the presence of normal levels of IE proteins.

In Example 1, the data presented establish that cellular cdks are required for HSV replication. In the experiments reported in the present Example 2, the data establish that at least two stages of the HSV replication cycle require cellular cdks, in that, accumulation of HSV immediate-early transcripts and accumulation of HSV early transcripts were dependent on cdk activity.

Inhibition of HSV transcription by Rosco when added 6 hours after infection, or after release of CHX inhibition, proves that the effects of Rosco on transcription, and hence on HSV replication, are not due to a block in the transport of the capsids to the nucleus or to a defect in uncoating. Moreover, Rosco does not appear to inhibit transcript accumulation by stimulating RNA degradation. The levels of two early (ICP8 and TK) and one late transcript remained stable for several hours after addition of Rosco under all conditions tested. On the other hand, the levels of ICP0, and to a lesser extent ICP4, transcripts decreased after addition of Rosco under certain circumstances. In the CHX-release experiments, the levels of ICP0 transcript at 9 hours after addition of Rosco were about 1/4 of the initial levels (estimated half-life, about 4.5 hours). The levels of ICP4 transcript decreased less than the levels of ICP0 transcript. The stability of the ICP0 and ICP4 transcripts in the presence of Rosco is consistent with their previously documented half-lives, estimated between 1.5 and 5 hours (Oroskar et al., 1989, J. Virol. 63 (5):1897-1906; Henley et al., 1991, Virus Research 20:121-132).

### EXAMPLE 3

### Roscovitine-Dependent Inhibition of HSV-1 Immediate Early Gene Expression

In order to measure the effects of Rosco on VP 16-dependent transcriptional activation during viral infection, a transient transfection/infection assay was utilized. Vero cells were transfected with 1 μg of a plasmid (pWRICP0-CAT) that contains the gene encoding the chloramphenicol acetyl transferase (CAT) gene under the control of the promoter regulatory region of the IE gene, ICP0. At 48 hours post-transfection, cultures were infected with the equivalent of 10 PFU/cell of UV-inactivated HSV-1, strain KOS, in the presence and absence of 100 μM Rosco. At 3,6, and 9 hpi, the cultures were harvested and CAT activity was measured. UV-inactivation of viral stocks inhibits viral gene expression following infection but leaves the activities of tegument proteins, including VP16, intact. Thus, in this assay, activation of the ICP0 promoter in the transfected plasmid by infection with UV-inactivated virus is mediated by VP 16 and possibly other virion-associated proteins.

The addition of Rosco at the time of infection blocked the ability of UV-inactivated KOS to induce CAT expression from pWRICP0-CAT (Table 2, lines 1-4). In the presence of Rosco, the level of CAT activity in virus-infected cultures (line 4) was similar to mock-infected cultures (line 1). In the absence of Rosco, the level of CAT activity in virus-infected cultures at 9 hpi was 39-fold higher than in mock-infected cultures (line 2). The addition of Rosco had no measurable effect on the basal level of CAT expression in mock-infected cultures (line 3). The results of these tests demonstrate that Rosco inhibits virion-induced IE gene expression.

Since Rosco inhibits HSV replication even when added to infected cells at 6 hours pi, it was of interest to determine if cdk activity was required for activation of HSV IE promoters at different times after infection, and to determine when IE gene expression is most sensitive to inhibition by the drug. For this purpose, Vero cells were transfected with 1 μg pWRICP0-CAT and were mock-infected or infected with 10 PFU/cell of UV-inactivated KOS at 48 hours post-transfection. The cultures were divided into 6 groups containing 6 dishes each. At 0, 2, 4, and 6 hours pi the culture medium in a single group was removed and replaced with medium containing 100 μM Rosco. In addition, at 0, 2, 4, 6, 8, and 10 hours pi, one dish from each group was harvested and CAT activity was measured. The mock-infected group was not treated with Rosco.

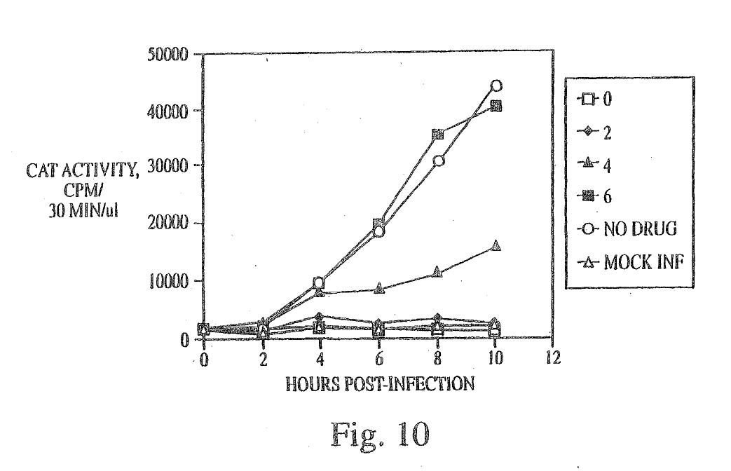

Inhibition of virion-induced CAT expression by Roscovitine was most efficient when drug was added at 0 and 2 hours pi (FIG. 10) in that the levels of CAT activity when Roscovitine were added at these times was similar to basal levels of CAT activity detected in mock-infected samples. Rosco was less effective in inhibiting virion-induced CAT activity when added at 4 hours pi. Notably, the level of CAT activity did not change significantly after Rosco addition at this time suggesting that the drug inhibited new CAT expression. By 6 hours pi, CAT activity in infected cultures was refractory to Rosco inhibition in that the levels of CAT activity in the presence of Rosco were comparable to those in the absence of drug. The results of these tests indicate either that 1) Rosco inhibits virion-induced activation of IE promoters at a step that occurs prior to 6 hours pi, or 2) by 6 hours pi, the level of CAT mRNA is saturating for translation in the infected cell and blocking synthesis of new CAT mRNA does not effect translation of the remaining CAT message.

Lovastatin and K252a do not inhibit virion-induced IE gene expression Rosco inhibits IE gene expression either by blocking cdk activity, or by blocking the activities of downstream proteins which are both activated by cdks and required for cell-cycle progression. To test whether cell-cycle inhibition or inhibition of other serine/threonine kinases blocks virion-induced IE gene expression, a well-characterized cell-cycle inhibitor, Lovastatin, and a broad spectrum serine/threonine kinase inhibitor, K252a, were tested for their ability to inhibit virion-induced CAT expression. These drugs have no effect on HSV-1 replication. Lovastatin is an HMG-CoA reductase inhibitor that blocks farnyslation of ras preventing its association with the plasma membrane. The interaction of ras with the plasma membrane is required to transduce growth factor-dependent signaling to the nucleus. Blocking this signaling pathway arrests cells in the G1 phase of the cell-cycle. Thus, although Rosco and Lovastatin inhibit cell-cycle progression, their mechanisms of action are quite different.

To test the effects of Lovastatin and K252a on HSV-1 E gene expression, Vero cells were transfected with 1 μg of pWRIC0-CAT. At 48 hours post-transfection, the cultures were infected with 10 PFU/cell of UV-inactivated KOS in the presence and absence of 100 μM Roscovitine, 10 μM Lovastatin and, 250 μM K252a. At 3, 6, and 9 hours pi, infected cultures were harvested and CAT activity was measured.

As shown in Table 2, Lovastatin (line 8) and K252a (line 6) had little effect on virion-induced activation of IE gene expression when added at the time of infection. Likewise, cultures treated with Lovastatin or K252a 24 hours prior to infection had no effect on virion-induced IE gene expression. Collectively, the results of the tests shown in Table 2 demonstrate that virion-induced activation of IE gene expression requires enzymes (most likely cdks) that are sensitive to inhibition by Roscovitine but not Lovastatin or K252a.

TABLE 2

| | | Fold-induction of CAT activity | | |
|---|---|---|---|---|
| | | Hours pi | | |
| Line | Treatment | 3 | 6 | 9 |
| 1 | Mock | 1.3 (0.6) | 0.9 (0.3) | 1.0 (0.3) |
| 2 | UV-KOS | 2.3 (1.3) | 17.7 (3.4) | 39.3 (5.1) |
| 3 | Rosco | 1.3 (0.6) | 0.8 (0.3) | 0.8 (0.3) |
| 4 | Rosco + UV-KOS | 0.8 (0.2) | 0.9 (0.4) | 0.9 (0.2) |
| 5 | Lova | 1.1 (0.6) | 1.0 (0.5) | 1.2 (0.6) |
| 6 | Lova + UV-KOS | 2.1 (1.2) | 21.4 (4.2) | 47.5 (6.0) |
| 7 | K252a | 0.8 (0.4) | 1.1 (0.3) | 1.0 (0.3) |
| 8 | K252a + UV-KOS | 2.3 (1.4) | 16.2 (3.7) | 26.6 (3.6) |

Table 2: Roscovitine, but not K252a or Lovastatin, inhibit virion-induced activation of a viral IE promoter. Vero cells (2×105/60 mm dish) were transfected with a 1 μg of plasmid pWRICPO-CAT. At 48 hours post-transfection the cultures were infected with the equivalent of 10 PFU/cell of UV-inactivated HSV-1, strain KOS, in the presence and absence of 100 μM Roscovitine. At 3, 6 and 9 hours pi, the cultures were harvested and CAT activity was measured. CAT activity was measured from three independent experiments and data are expressed as fold-activation relative to mock-infected cultures at the 3 hour time point. The numbers in parenthesis represent the standard error of the mean.

### EXAMPLE 4

### Roscovitine Inhibits Herpes Simplex Virus DNA Replication in the Presence of Viral Early Proteins

The data presented in Examples 1, 2 and 3 establish that Rosco and Olo inhibit replication of HSV, and hence HSV replication requires the activities of cdks. Further, the data establish that at least two distinct steps in the viral replication cycle require cdks: transcription of IE and transcription of E genes. Since it has not been possible to isolate HSV mutants resistant to Rosco and Olo, cdks may be also required for other viral functions. In view of the fact that when Rosco was added to infected cells at 6 hours pi HSV replication was efficiently inhibited, it seemed that viral functions that occur after E gene expression may also require cdk activity and hence should be sensitive to inhibition by Rosco. In the experiments presented in the present Example, the efficiency of viral replication, expression of viral genes, and viral DNA replication during infections in which Rosco was added after E proteins were expressed was measured. Rosco inhibited HSV replication, and specifically viral DNA replication, when the drug was added at the time of release from a 12 hour PAA-induced block in DNA replication. Under these conditions, Rosco had no effect on steady-state levels of two E transcripts. Inhibition of viral DNA replication was shown not to be a consequence of inhibition of expression of viral proteins in that Rosco inhibited viral DNA replication even in the absence of new protein synthesis. In a second series of experiments, Rosco added at the time of shift-down from the non-permissive to the permissive temperature inhibited viral DNA replication of four HSV mutants harboring temperature sensitive (ts) mutations in genes essential for viral DNA replication. Expression of E genes was not inhibited by Rosco under these conditions. Based on these findings, it can be concluded that cellular cdks sensitive to inhibition by Rosco are required for replication of viral DNA, even in presence of viral E proteins. This requirement may reflect the fact that HSV DNA replication is functionally linked to transcription, which requires cdks, or it may indicate that both viral transcription and DNA replication, independently, require viral or cellular factors activated by cdks.

Cells, Virus, Plasmids and Drugs

The preparation of cells, virus, plasmids and drugs was as described in the Examples presented herein. Final concentrations of drugs were 100 or 400 μg/ml PAA, as indicated in the text and description of the figures, and 100 μM Rosco and 50 μg/ml CHX were used in all of the relevant experiments.

Vero cells (2×10⁵ cells) in 12 well plates were infected at the indicated multiplicities with HSV-1 strain KOS. Where indicated, medium overlaying infected cells was replaced with fresh drug-containing or control drug-free medium. Viral titers at specific times post infection were determined as previously described herein.

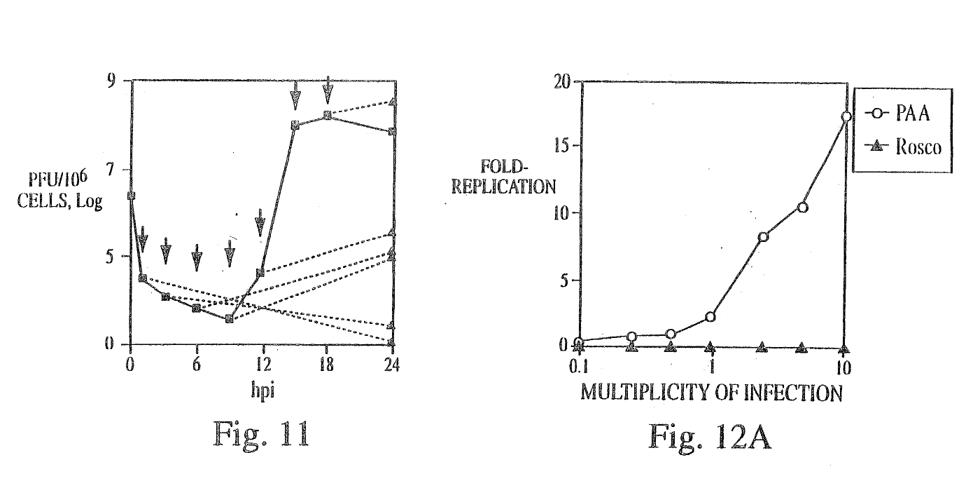

In the case of those experiments in which the time of addition of Rosco was varied (FIG. 11), drug-free medium was removed from infected cells at the indicated times post infection and replaced with two volumes of drug-containing medium. Two volumes of medium were used in order to dilute any residual drug-free medium remaining on the monolayers after the washes.

In the case of drug-replacement and drug-release experiments, drug-containing medium was removed from infected monolayers at the indicated times post-infection. Infected cells were then washed twice with PBS containing the same concentration of drug to be added to the cultures after washing. Afterward, two volumes of drug-free medium, or medium containing CHX, PAA, and/or Rosco were added to each monolayer. Two volumes of medium were used in order to dilute any residual drug remaining on the monolayers after the washes.

In the case of temperature shift-down experiments, the indicated ts mutants were used to infect Vero cells at the non-permissive temperature (39.5° C.). Viral inoculum, prepared as described herein, was pre-warmed to 39.5° C. immediately before addition to cells. After 1 hour adsorption at 39.5° C., inoculum was removed and fresh medium, pre-warmed to 39.5° C., was added to infected monolayers. For release in the presence of Rosco or PAA, the medium overlaying infected monolayers was replaced at 5 hours pi with fresh medium containing the indicated drug pre-warmed to 39.5° C. immediately prior to use. After 1 hour at the non-permissive temperature in the presence of drug, dishes containing infected cells were transferred to the permissive temperature (34° C.), and maintained at this temperature until harvested.

Probes, RNase protection and viral DNA replication assays Riboprobes were synthesized as described herein. RNase protection assays were performed using the DirectProtect kit (Ambion), and DNA was extracted and evaluated by slot blot hybridization as described herein.

Metabolic Labeling

For metabolic labeling experiments, cells were infected with 6 PFU/cell of t Al 5 at the non-permissive temperature and shifted down to the permissive temperature in methionine-free medium containing no drug or PAA (100 or 400 μg/ml, as indicated) or Rosco (100 μM), together with 1 μl/ml of ³⁵S-methionine.

Rosco Inhibits HSV Replication when Added to Infected Cells at 3, 6, 9. or 12 Hours pi

As a first step in determining whether Rosco-sensitive cdks are required for an essential viral function that occurs after E proteins are expressed, it was determined whether addition of Rosco at 3 hour intervals after infection inhibited HSV replication. If Rosco inhibits only expression of IE and E gene products, addition of the drug after 6 hours pi would have no major effect on HSV replication. In contrast, if the drug inhibits an essential viral function that occurs after E gene products have been expressed, Rosco should inhibit HSV replication even when the drug is added after 6 hours pi. To address this question, Vero cells were infected with 2.5 PFU/cell of HSV-1 and medium was removed from an infected monolayer every 3 hours pi and replaced with medium containing 100 μM Rosco.

As described herein, addition of Rosco before 6 hours pi resulted in a significant block in HSV replication (FIG. 11). Interestingly, addition of Rosco at any time after 6 hours pi, but before completion of a single-step growth cycle, also inhibited HSV replication (FIG. 11). It is clear from FIG. 11 that inhibition of viral replication was almost total when Rosco was added to infected monolayers at 1 or 3 hours pi, whereas inhibition was only partial when the drug was added at 6 hours pi or later. Nonetheless, when Rosco was added at 6, 9, or 12 hours pi, HSV titers at 24 hours pi were approximately 2.5 orders of magnitude lower than the titers obtained in untreated cultures. Thus, Rosco inhibited HSV replication even when added under conditions in which E gene products should have been present (i.e., at 9 or 12 hours pi).

Rosco Inhibited HSV Replication After Release from a 12 Hour PAA Block

Based on the findings presented in FIG. 11, it was hypothesized that cellular cdks are required for essential viral replication functions that occur after E proteins are expressed. Because viral DNA replication occurs immediately after, or concomitant with E gene expression, it was hypothesized that viral DNA replication could be one of the essential viral functions that require cdks. As a first test of this hypothesis, it was determined whether Rosco can inhibit HSV replication after release of a PAA block. PAA is a well-characterized inhibitor of HSV DNA polymerase. In the presence of PAA, viral IE and E proteins are expressed but the activity of the viral polymerase (and E protein) is directly inhibited by the drug. Consequently, E proteins are expressed but viral DNA is not replicated.

Because high doses of PAA cause irreversible inhibition, relatively low doses must be used for the block in DNA replication to be reversed. However, the efficiency of inhibition of HSV replication by PAA is also multiplicity-dependent because PAA targets a virally encoded protein. This multiplicity-dependence is based on the fact that the number of gene copies, from which the DNA polymerase is expressed, increases with increasing multiplicities. Consequently, the levels of the target of PAA inhibition (i.e., viral DNA polymerase) also increase significantly at high multiplicities. The optimal multiplicity to be used in PAA reversal experiments was therefore determined. Rosco was included in these experiments for comparison because Rosco targets cellular proteins whose levels of expression should not be grossly affected by the multiplicity. Thus, in contrast to the inhibition of HSV replication by PAA, which is multiplicity-dependent, inhibition of HSV replication by Rosco was expected to be relatively independent of multiplicities.

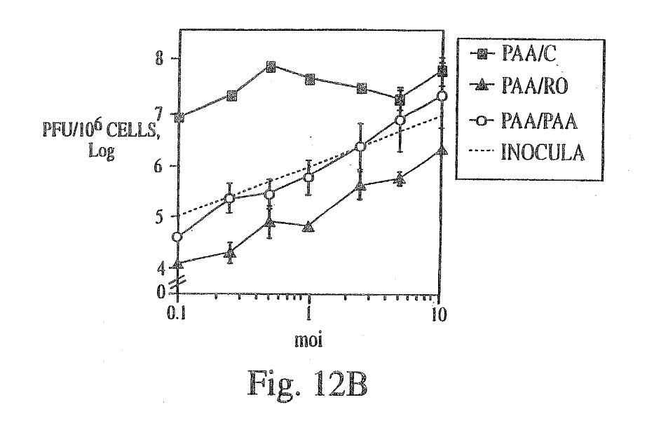

Vero cells were infected with 0.1 to 10 PFU/cell of HSV-1. After adsorption, infected cells were overlaid with medium containing 100 μg/ml of PAA, 100 μM of Rosco, or no drug (control). Twenty-four hours after infection, cells were harvested and titers of infectious virus were measured by standard plaque assay. As shown in FIG. 12A, PAA inhibited HSV replication efficiently at multiplicities of 1 PFU/cell or lower, whereas at the concentration used in these experiments PAA inhibited viral replication less efficiently if the multiplicity was 2.5 PFU/cell or higher. In contrast, Rosco inhibited HSV replication efficiently at all multiplicities tested (FIG. 12A).

Next, we determined whether Rosco inhibits viral replication after release from a 12 hour PAA block. For this purpose, Vero cells were infected at multiplicities ranging from 0.1 to 10 PFU/cell. After adsorption, all infected monolayers were overlaid with medium containing 100 μg/ml PAA. At 12 hours pi, PAA-containing medium was replaced with fresh medium containing the secondary drug (PAA or Rosco) or no drug (Control) and 24 hours later (36 hours pi), cells were harvested and infectious virus was tittered by standard plaque assays. As shown in FIG. 12B, Rosco added at the time of release from a PAA block inhibited viral replication efficiently. In fact, at all multiplicities tested, Rosco inhibited viral replication slightly more efficiently than PAA, a well-characterized direct inhibitor of HSV DNA polymerase.

Inhibition of HSV DNA Replication by Rosco Added at the Time of Release From a 12 Hour PAA Block is Multiplicity-Dependent

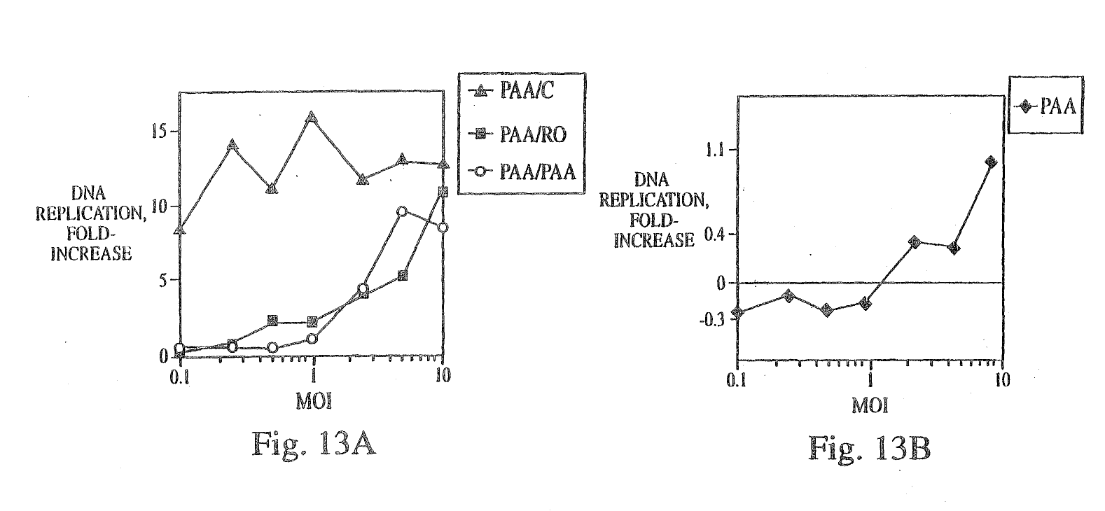

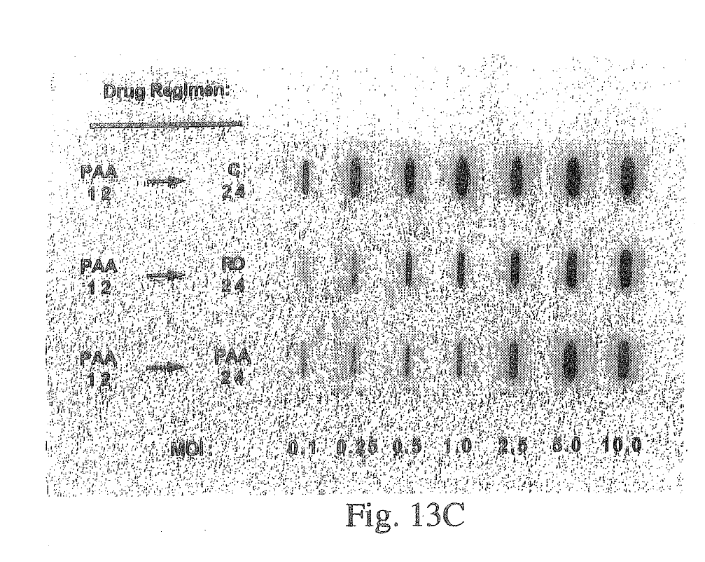

The level of HSV DNA replication that occurs after release from a 12 hour PAA block was next determined. Vero cells were infected with 0.1 to 10 PFU/cell of HSV-1, and infected cells were treated with PAA for 12 hours and released as described herein. Immediately before or 24 hours after the change of medium, cells were harvested and DNA was extracted and quantitated as described herein. As expected from the results presented in FIG. 12, when virus replication was blocked by PAA for 12 hours, and then medium was changed to fresh PAA-containing medium, the extent of inhibition of HSV DNA replication after the change of medium was dependent upon the multiplicity of infection (FIGS. 13A and 13B). Similarly, Rosco inhibited HSV DNA replication efficiently after release from the 12 hour PAA block when the moi was 1 PFU/cell or lower. When the moi was of 2.5 PFU/cell or higher, Rosco inhibited viral DNA replication less efficiently after release (FIGS. 13A and 13B). Importantly, at all multiplicities tested the extent of inhibition of HSV DNA synthesis by Rosco was similar to the extent of inhibition achieved by PAA, suggesting that Rosco also inhibits, directly or indirectly, proteins required for viral DNA replication.

HSV DNA replication was inhibited to similar degree by PAA or Rosco, after release from a 12 hour PAA bloc, the question of whether the extent of viral DNA replication after release from the PAA block correlated with the extent of viral DNA replication during the 12 hour block was evaluated. For this purpose, Vero cells were infected with varying multiplicities of KOS and treated with PAA for 12 hour as described above. One set of infected monolayers was harvested immediately after adsorption and a second set was evaluated at 12 hours pi. DNA was extracted from the harvested cells and the fold increase in viral DNA replication during the 12 hour block in PAA was evaluated by slot blot analysis. As for the inhibition of viral replication at 24 hours pi (FIG. 13B), the extent of inhibition of HSV DNA synthesis during the 12 hour block in 100 μg/ml of PAA was found to be dependent upon the multiplicity of infection (FIG. 13C). Thus, HSV DNA was not replicated during the 12 hour PAA block when the multiplicity was 1 PFU/cell or lower. When the multiplicity was 2.5 PFU/cell or higher, however, inhibition of HSV DNA replication by PAA was only partial (FIG. 13C). Based on these data, it was concluded that the level of viral replication after release from the 12 hour PAA block (in the presence of Rosco or PAA) correlates with the extent of DNA replication during the 12 hour block.

Rosco did not Reduce Levels of E Transcripts when the Drug was Added After Release from a 12 Hour Block in PAA

It was possible that Rosco-induced inhibition of HSV DNA replication after release from a 12 hour PAA block may have been secondary to a decrease in the levels of the viral proteins required for DNA synthesis. Because viral proteins were not recovered in amounts that allowed reliable quantitations at low multiplicities, the levels of viral transcripts were assessed as an indirect measure of the level of viral proteins in the cells. Rosco does not inhibit translation of viral proteins once the mRNAs have been expressed.

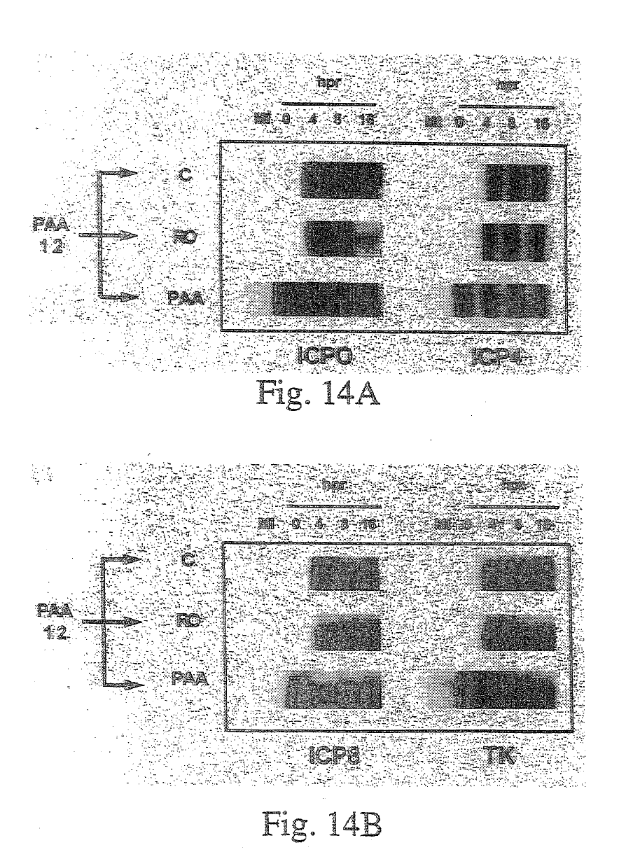

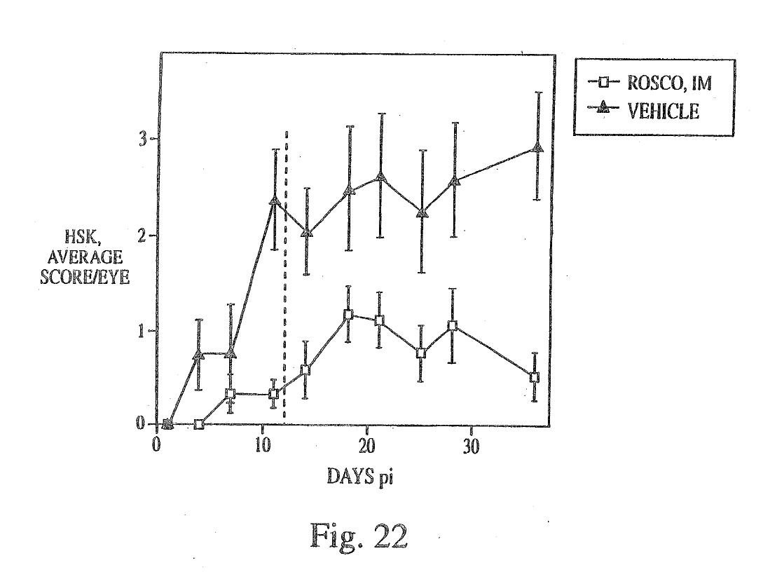

Vero cells were infected with 2.5 PFU/cell of HSV-1, infected cells were incubated in the presence of PAA and were released from the PAA block into no drug (C), PAA, or Rosco (FIG. 14A) as described herein. Immediately before (0 hours) or 4, 8, or 16 hours after changing the medium, cells were harvested and RNA was extracted and quantitated using RNase protection assays as described herein. A multiplicity of 2.5 PFU/cell was selected because it was the highest multiplicity at which Rosco efficiently inhibited viral DNA replication after release from a 12 hour PAA block (FIG. 22).

Following release from the 12 hour PAA block, steady-state levels of transcripts of the IE gene encoding ICP0 were maintained at approximately the same levels if the infected cells were kept in PAA, or increased slightly if the infected cells were released in control medium (FIG. 14A). In contrast, steady-state levels of the ICP0 transcripts decreased after addition of Rosco, and the transcripts exhibited a half-life consistent with the known half-life of this transcript (FIG. 14A). This situation was similar to that when Rosco was added at the time of release from a 6 hour block in CHX. Steady-state levels of the transcripts of two E genes (those encoding ICP8 and TK) did not change significantly after release from the 12 hour PAA block in the presence of PAA or Rosco, or in the absence of any drug. Thus, when Rosco was added after release of a PAA block no dramatic effect on the levels of the transcripts of two E genes was observed.

Rosco Inhibited HSV DNA Replication when Added After Release from a PAA Block, Even in the Absence of New Protein Synthesis

A reversible PAA block can be achieved only at relatively low multiplicities. Some viral DNA replication proteins are expressed at very low levels (e.g., the catalytic subunit of viral DNA polymerase and the origin binding protein (OBP)). Thus, it was not possible to evaluate the levels of all viral proteins required for DNA replication in the cells released from the PAA block. As an alternative, HSV DNA replication was evaluated following release from a PAA block in the presence of CHX and PAA or Rosco. Addition of CHX at the time of release from a PAA block resulted in inhibition of new viral protein synthesis. Therefore, in this experimental design, viral DNA was replicated after release of the block in PAA using viral proteins which were synthesized before release. Consequently, any potential effect of Rosco on viral gene expression would be irrelevant. If the inhibition of viral DNA synthesis produced by Rosco in the PAA-release experiments was exclusively a consequence of inhibition of E gene expression, Rosco should not inhibit viral DNA synthesis beyond the inhibition resulting from the addition of CHX at the time of release. In contrast, if inhibition of viral DNA synthesis was at least partially due to a requirement for enzymes that are sensitive to Rosco inhibition, CHX and Rosco added together should inhibit viral DNA synthesis to a larger extent than CHX alone. Thus, PAA release experiments were performed in which no drug, PAA, Rosco, CHX, Rosco and CHX, or PAA and CHX were added to infected cells at the time of release.

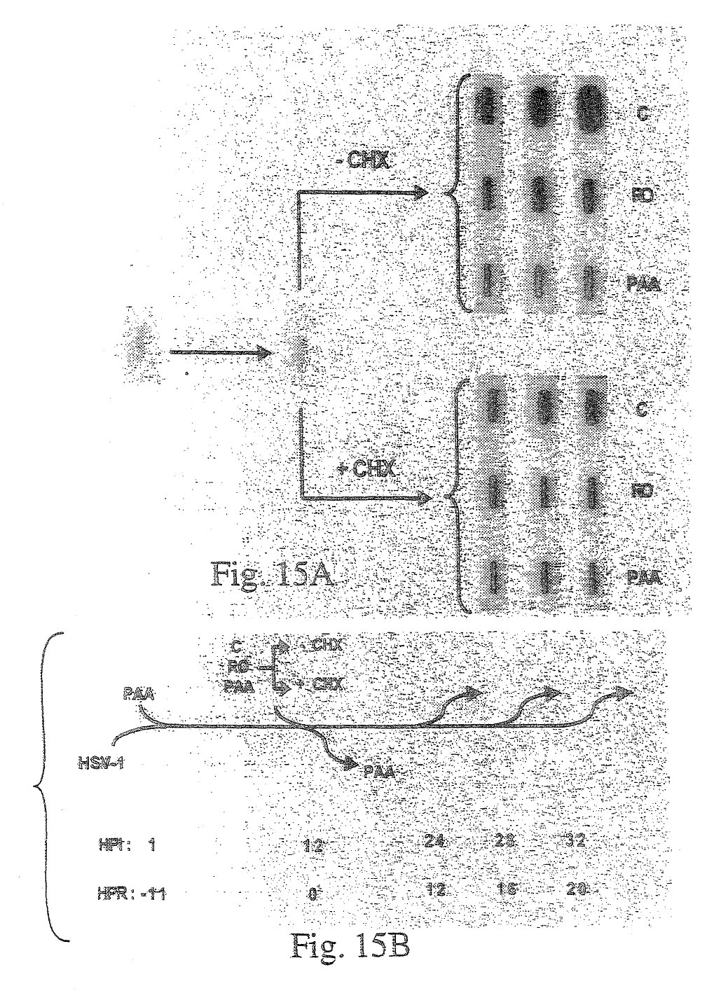

Two sets of Vero cells were infected with HSV-1, treated with PAA and released from the PAA block as described herein. The first set of infected monolayers was released from the PAA block in medium containing no drug, 100 μM Rosco, or 100 μg/ml PAA in the absence of CHX (FIG. 15A). However, the medium added to the second set at the time of release contained 50 μg/ml of CHX in addition to the secondary drug (Rosco or PAA) (FIG. 15B). As a control, a subset of monolayers was released from the PAA block in the presence of CHX in the absence of PAA or Rosco (FIGS. 15B and 15C). One infected monolayer was harvested immediately before release to analyze the levels of viral DNA at the time of release. Individual monolayers in each of the two sets of released dishes were then harvested at 12, 16, or 20 hour post-release and the extent of viral DNA replication that occurred after release from the PAA block was determined by slot blot analysis.

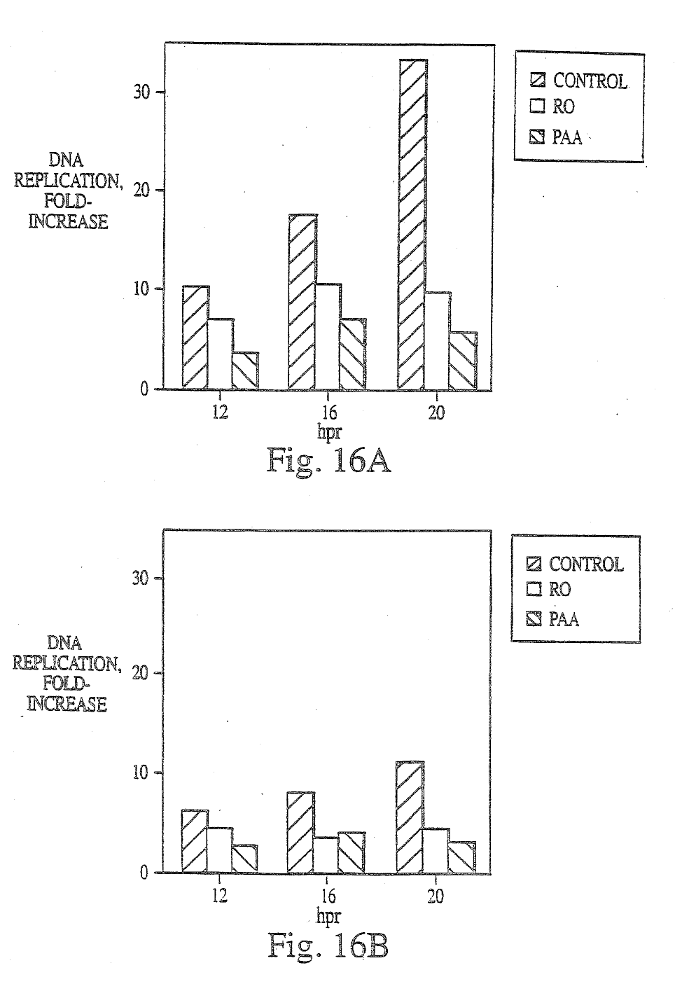

After release of the PAA block in the absence of secondary drug, viral DNA had replicated approximately 10-fold within the first 12 hours, and viral DNA replication continued until 20 hours post-release (FIGS. 15A and 16B), when viral DNA had replicated approximately by 35-fold. As expected, if the infected monolayers were maintained in PAA, only low levels of DNA replication (approximately 7-fold) were observed within the 20 hour period after the change from the primary to the secondary drug. When infected monolayers were released in the presence of Rosco, viral DNA replicated only slightly more efficiently than in the presence of PAA (approximately 10-fold versus approximately 7-fold). Interestingly, most DNA replication in the presence of Rosco occurred in the first 12 hours post-release (FIGS. 16A and 16B).

When release from the PAA block was performed in the presence of CHX (FIGS. 16A and 16B), relatively low levels of viral DNA replication were achieved because of the absence of new protein synthesis. Thus, viral DNA replicated approximately 6-fold in the first 12 hours post-release, and approximately 12-fold by 20 hours post release. However, viral DNA synthesis was inhibited even further if PAA was added together with CHX at the time of release from the primary PAA block. Thus, viral DNA replicated only about 5-fold during the 20 hours after release which were examined. Interestingly, when the release was performed in the presence of Rosco and CHX, viral DNA synthesis was inhibited to a similar level (about 5-fold) than when the release was performed in the presence of CHX and PAA, a drug known to directly inhibit the viral DNA polymerase.

These data establish that Rosco inhibited viral DNA replication after release from a PAA block, and this inhibition was not mediated by inhibition of viral gene expression.

Rosco Inhibited Replication of HSV ts Mutants when Added at the Time of Release from a 6 Hour Block at the Non-Permissive Temperature

To confirm the above findings, the viral DNA replication requirement for cellular cdks was further evaluated. Four HSV-1 ts mutants, A1, A15, D9, and P23 were used in these experiments. The ts mutations in A1 and A15 have been mapped to the ICP8 gene, and the mutation in D9 has been mapped to the catalytic subunit of the viral DNA polymerase. The mutation in P23 has not been mapped precisely, but available evidence indicates that it too maps to the viral DNA polymerase gene.

Vero cells were infected at 39.5° C. with each of the ts mutants and infected cells were maintained at the non-permissive temperature for 6 hours. At this time, viral IE and E genes have been expressed, yet little or no viral DNA has been synthesized. At 6 hours pi, one set of monolayers infected with each of the mutants was harvested, and two other sets of monolayers were shifted-down to the permissive temperature. Of these last two sets of monolayers, one was shifted-down in control medium containing no drug and the other was shifted down into Rosco-containing medium. Viral replication was evaluated 24 hours after shift-down in standard plaque assays.

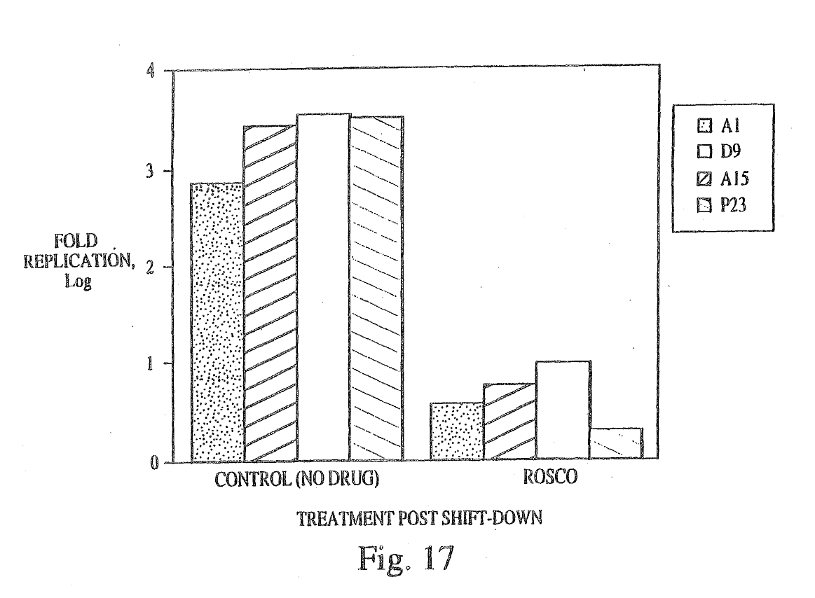

In FIG. 17 there is shown efficient Rosco mediated inhibition of replication of the four ts mutants. Thus, when cells infected with ts A15, D9, or P23 mutants were shifted-down to the permissive temperature in the presence of Rosco at 6 hours pi, viral replication 18 hours later (i.e., 24 hours pi) was approximately 3 orders of magnitude lower than when infected cells were shifted down in the absence of drug. During infections with ts A1, the difference in titers at 24 hours pi between infections shifted-down in the presence or absence of Rosco was only two orders or magnitude, primarily because the reversal of the temperature shift-down with this mutant is not as effective as the temperature reversal of the other mutants tested (FIG. 17).

Rosco Inhibited HSV DNA Replication of ts A15 Following Temperature Shift-Down in the Presence of the Drug

It was next determined whether the inhibition of viral replication observed in the experiments reported above was mediated by inhibition of viral DNA synthesis. For these experiments, ts mutant A15 was selected because the mutation in this strain has been mapped, reversal of the shift-down is efficient, and because Rosco inhibited the replication of this virus mutant efficiently after shift-down (FIG. 17). A multiplicity of 6 PFU/cell was selected because it was necessary to examine expression of E gene products and viral DNA synthesis in parallel. It had been determined that 6 PFU/cell is the lowest multiplicity that permits reliable quantitation of E protein synthesis.

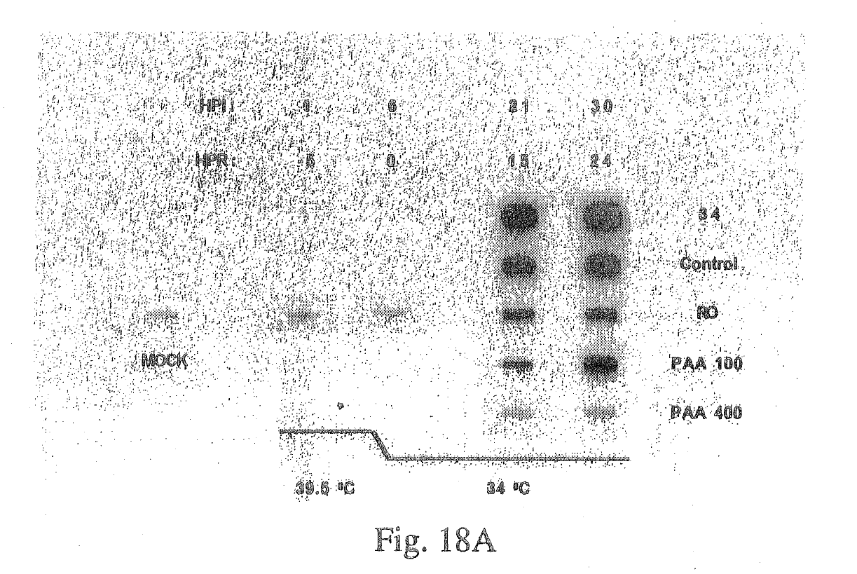

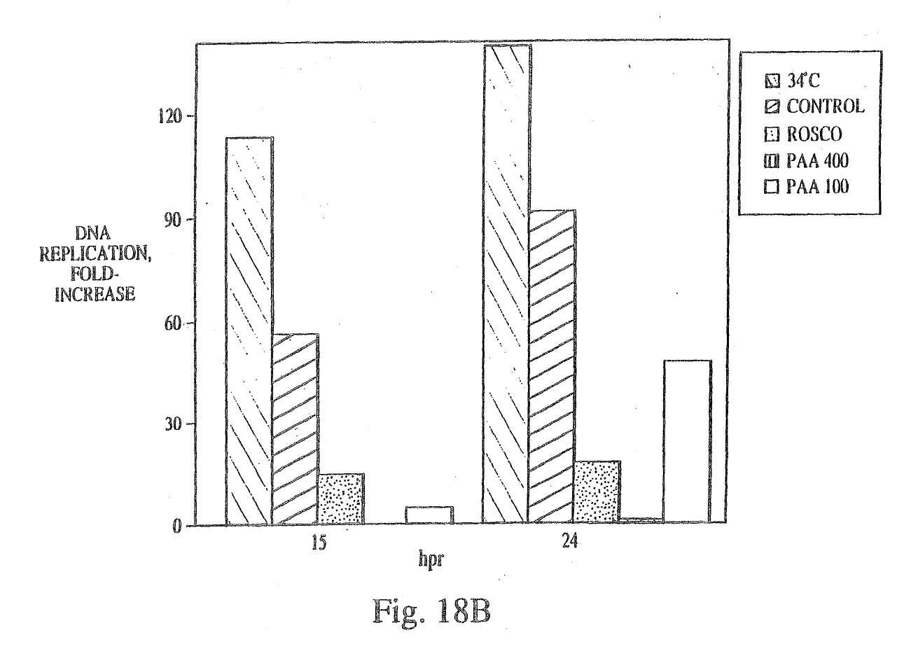

Vero cells were infected with ts A15 at the non-permissive temperature and infected cells were shifted-down at 6 hours pi as described herein. At the indicated times before or after shift-down, infected monolayers were harvested, DNA was extracted, and the extent of viral DNA replication was evaluated by slot blot analysis. For comparison, a set of monolayers was infected and maintained at the permissive temperature throughout the experiment (FIG. 18, 34° C.). When shift-down was performed in the absence of any drug, viral DNA replicated approximately 60-fold in the first 15 hours and approximately 95-fold at 24 hours post-release (FIG. 18, Control). At 24 hours post-shift-down, levels of viral DNA in cells infected at the non-permissive temperature and then shifted down in control medium reached approximately 70% of the levels of viral DNA present in cells that had been infected and maintained at the permissive temperature throughout the 30 hour experiment (FIG. 18A). In contrast, when cells were shifted-down in the presence of 400 μg/ml PAA, viral DNA replication was totally inhibited during the 24 hour period after release. When infected monolayers were shifted-down in the presence of 100 μg/ml of PAA, viral DNA replication was significantly delayed, but not totally inhibited. Thus, under these conditions, viral DNA replicated only about 5-fold in the first 15 hours after shift-down, but almost 50-fold by 24 hours after shift-down, to reach about 35% of the level of viral DNA attained in cells infected and maintained at the permissive temperature. When the shift-down was performed in the presence of Rosco, viral DNA replicated only about 18-fold, to reach about 12% of the level attained in cells infected and maintained at the permissive temperature. As observed in the PAA-release experiments, most of the viral DNA replication in the presence of Rosco occurred in the first 15 hours after shift-down (post-release).

These data establish that Rosco inhibited viral DNA synthesis significantly when cells infected with ts A15 were shifted-down from the non-permissive to the permissive temperature.

Rosco did not Inhibit HSV E Gene Expression when ts Mutants were Released from a 6 Hour Block at the Non-Permissive Temperature in the Presence of the Drug

If inhibition of viral DNA synthesis by Rosco after shift-down was exclusively because of inhibition of E gene expression, levels of E gene products should be significantly inhibited under the same conditions in which viral DNA synthesis is inhibited. If inhibition of viral DNA synthesis is at least partially mediated by inhibition of post-translational modification of specific E proteins, levels of E gene products may not be significantly inhibited under the conditions in which viral DNA synthesis was inhibited. To test this hypothesis, the levels of two E transcripts and a number of E proteins were evaluated. Specifically, transcripts of the genes encoding ICP8, because it is the gene which contains the mutation in ts A15, and TK, because it is an E protein involved in a different aspect of viral DNA replication than is ICP8, were evaluated.

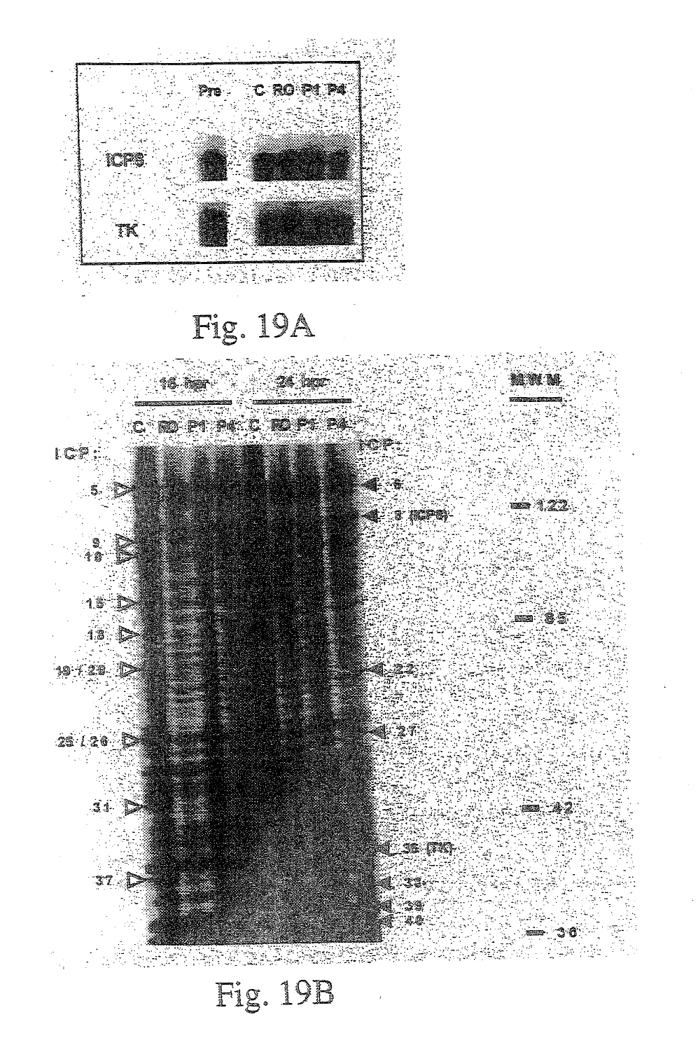

Vero cells were infected at 39.5° C. with 6 PFU/cell of ts A15 and were incubated at the non-permissive temperature for 6 hours. One hour before shift-down to the permissive temperature, 100 μM Rosco, 100 μg/ml or 400 μg/ml PAA was added to an infected monolayer, whereas a fourth monolayer was maintained in drug-free medium (control). Sixteen hours after shift-down, cells were harvested and levels of ICP8 and TK transcripts were measured in RNase protection assays. Infected cells in one dish were harvested before shift-down, to establish the levels of ICP8 and TK RNAs before shift-down (FIG. 19A, Pre).

As expected given the kinetics of E gene transcription, levels of ICP8 and TK mRNA at 16 hours post-release in control medium containing no drug were lower that the levels detected immediately before shift-down (FIG. 19A). Also as expected, the addition of 100 μg/ml of PAA did not significantly alter accumulation of transcripts of these two E genes. Notably, the addition of 400 μg/ml PAA or Rosco at the time of shift-down resulted in a slight increase in the steady-state levels of the two E transcripts analyzed. Collectively, these results do not support the hypothesis that inhibition of viral DNA replication by Rosco added at the time of shift-down was mediated by inhibition of accumulation of E transcripts.

Having shown that the steady-state levels of mRNA transcribed from the gene that contains the ts mutation was slightly higher, rather than lower, in cells shifted-down in the presence of Rosco than in the presence of PAA or in the absence of drug, expression of viral proteins was next analyzed. For this purpose, Vero cells were infected with 6 PFU/cell of mutant ts A15, maintained at the non-permissive temperature, and shifted-down as described herein. The culture medium added at the time of shift-down contained ³⁵S-methionine to evaluate viral protein synthesis after shift-down. Infected cells were harvested at 16 and 24 hours post-shift-down, and levels of viral proteins were evaluated in an SDS-polyacrylamide gel. Metabolic labeling was chosen over Western blot or immuno-precipitation because it allows the simultaneous observation of many viral proteins of different kinetic classes.

When the shift-down from non-permissive to permissive temperatures was performed in control medium containing no drug, accumulation of high levels of L proteins was evident at 16 or 24 hours post-release (FIG. 19B). Moreover, E proteins, including ICP8 and TK, were also clearly visible at 16 or 24 hours post release, because in this experimental design cumulative protein synthesis is evaluated. As expected, addition of 400 μg/ml of PAA at the time of shift-down resulted in significant inhibition of accumulation of L proteins, without a major effect on the levels of E proteins, whereas PAA at 100 μg/ml was not as effective as it was at 400 μg/ml. When Rosco was added at the time of shift-down, significantly lower levels of L proteins accumulated at 16 or 24 hours post release (FIG. 19B). In contrast, Rosco had little or no measurable effect on the level of E proteins, including ICP8 and TK. These results are also inconsistent with the hypothesis that Rosco-mediated inhibition of viral DNA replication after shift down is mediated exclusively by inhibition of expression of E proteins.

Collectively, the results of these studies demonstrate that in addition to transcription of IE and E genes, HSV DNA replication also requires cellular cdks.

### EXAMPLE 5

### Effects of cdk Inhibitors on HSV-1-Induced Stromal Keratitis (HSK)

Inhibition of HSV-1 Replication in vivo by Rosco

The data presented in the Examples thus far establish that HSV-1 replication in vitro is inhibited by Rosco and Olo. To test whether cdk inhibitors had any effect on herpes stromal keratitis (HSK), whose pathology is triggered by HSV infection and mediated by the host immune response to the infection, the following experiments were performed.

To determine whether Rosco inhibited HSV replication and the pathology associated with this virus in vivo, ICR mice were infected in each eye with 10⁵ PFU/eye of HSV-1, strain KOS, contained in 2.0 μl of inoculum, following corneal scarification. Specifically, mice were anesthetized, corneas were scarified 12 times horizontally and 12 times vertically with a 26G1/2 needle, and the inoculum was deposited in each eye. After infection, mice were randomly assigned to four groups containing 5 animals each. Starting at 7 hours post infection (pi), mice were treated twice daily with drug until day 4 pi (at 9 AM and 5 PM). Mice in the first group were treated with 71 μg/eye of Rosco suspended in 2.0 μl of mineral oil. Mice in the second group were treated with 35.5 μg/eye of Rosco suspended in 2.0 μl of mineral oil. Mice in the third group were treated with 17.7 μg/eye of Rosco suspended in 2.0 μl of mineral oil. Mice in the fourth (Control) group received 2.0 μl of mineral oil/eye. Every day, before the morning treatment (16 hours after the last treatment), eyes were swabbed and viral replication was monitored in standard plaque assays.

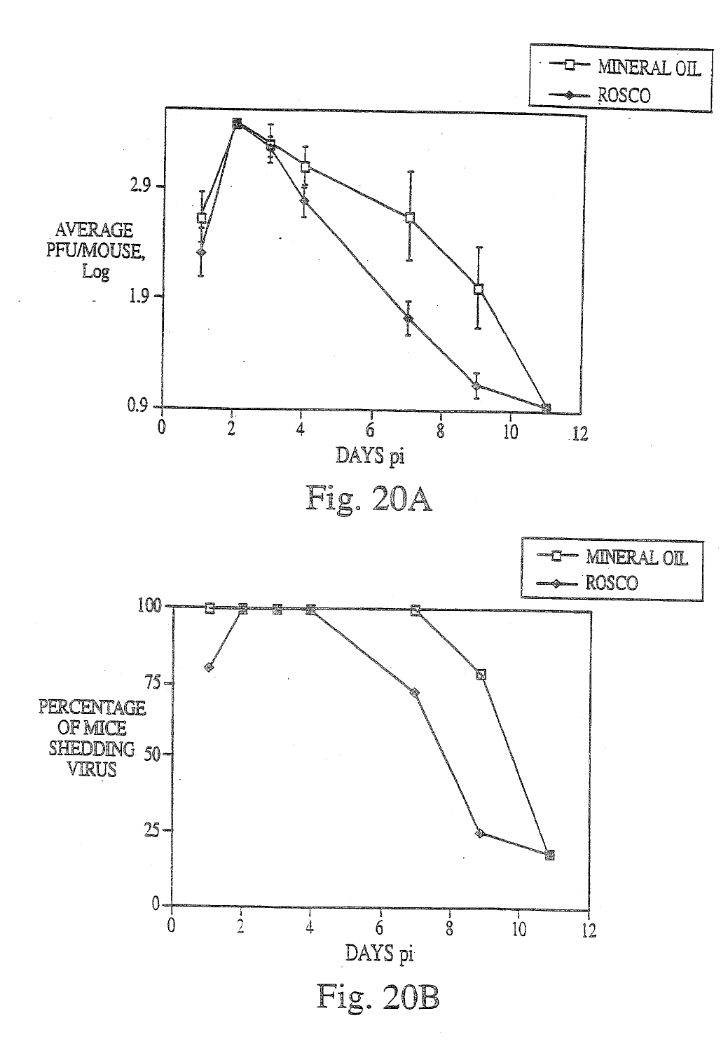

All three doses of Rosco inhibited HSV replication and no differences in the inhibitory effect of the three doses of the drug were observed. Consequently, the data obtained from mice receiving the three doses were pooled for analysis. After a lag period of three days, Rosco treatment significantly inhibited HSV-1 replication in the eye (FIG. 20). Thus, after day 4, Rosco-treated mice (15 animals) shed approximately 10-fold less virus than control animals (FIG. 20A). Moreover, by day 7 pi more than 25% of the treated animals had stopped shedding virus and by day 9 pi more than 75% had done similarly, whereas all control mice were shedding virus on day 7 pi, and 4 of 5 of them continued to shed significant amounts of virus on day 9 pi (FIG. 20B).

Rosco-Mediated Inhibition of the Development of HSV-Induced Stromal Keratitis After Challenge with a Standard Dose of HSV-1.

Because Rosco inhibited HSV-1 replication in vivo, the effect of this drug on the pathogenesis of HSK was examined. For this purpose, C.AL.20 mice (which are highly susceptible to the development of HSK after infection with HSV-1 were infected in the eye with 5×10⁵ PFU/eye of HSV-1, strain KOS, contained in 2.0 μl of serum-free medium (D-MEM), following corneal scarification. Mice were anesthetized, the periphery of the corneas was scarified as described above, and virus inoculum was deposited in each eye. After infection, mice were randomly assigned to two groups each containing 5 animals. Starting at 7 hours pi, mice in the treatment group were treated twice daily (at 9 AM and 5 PM) for 11 days by intramuscular (IM) injection of 11.33 μg (22.66 μg for the first treatment) of Rosco suspended in 300 μl of serum-free D-MEM (freshly prepared from 100 μM stock in DMSO). All mice were evaluated clinically in a blind manner twice a week. The standard scoring system shown in Table 3 was used to grade the degree of corneal opacity. Daily manipulation of animals exacerbated the symptoms of HSK, as manifested by more rapid onset of the disease and/or significant enhancement of the corneal pathology.

TABLE 3

| Score | Description |
|---|---|
| 0 | No lesion; cornea totally transparent. |
| 1 | Minor opacity; opacity difficult to detect |
| 2 | Clear opacity; opacity easy to detect |
| 3 | Severe opacity; iris difficult to see through the cornea |
| 4 | Very severe opacity; iris no longer visible through the cornea |
| 5 | Necrosis, hemorrhage, and/or scar tissue replacing the corneal |
| | stroma. |

Table 4: Scores used to evaluate the degree of corneal opacity. At the indicated times, mice were anesthetized and evaluated with a stereoscopic microscope using a standard scoring system. When possible, approximately one third of the mice were evaluated twice to confirm the consistency of the scoring system. Differences between the two readings were never more than one grade.

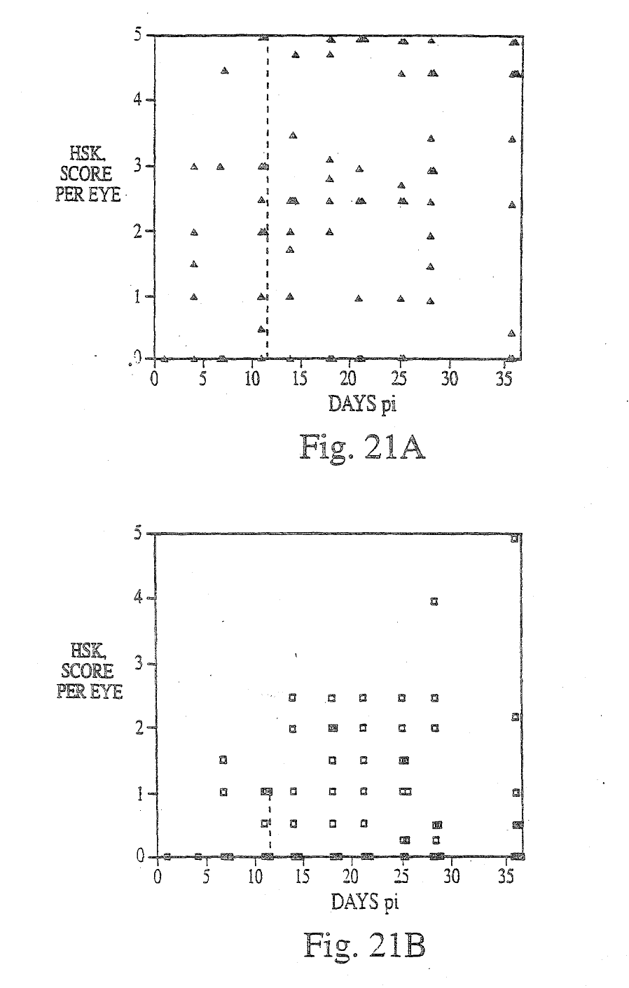

The data shown in FIGS. 20 and 21 demonstrate that the development of HSK was significantly inhibited by Rosco treatment during the 11 day treatment period. Thus, at day 11 pi, 5 corneas of mice in the control group had developed opacity which scored above 2. In contrast, none of the corneas of the mice in the treatment group had developed significant opacity (i.e., which scored above 2) (FIG. 21). After treatment was terminated on day 11, the majority of treated animals continued to be protected for at least 25 additional days. However, after stopping treatment, more than half of the eyes of the mice treated with Rosco developed a modest degree of corneal opacity, which was transient in most cases. Only the cornea of one eye in one mouse in the treatment group developed opacity which scored above 2 on day 36 pi. In contrast, in the control group the majority of the corneas had already developed some degree of opacity before treatment was stopped on day 11 pi. Moreover, by the end of the experiment, only three corneas of the mice in this group exhibited minor lesions, while all others corneas exhibited lesions ranging from fairly to extremely severe (which scored above 2). In five (half) of the corneas of mice in the control group, corneal tissue was so severely damaged that the corneas were totally opaque.

The average score per cornea and the standard error of the mean (SEM) for the data presented in FIG. 21 are shown in FIG. 22. These data provide more definitive evidence that development of HSK is significantly inhibited by Rosco. Thus, by day 4 pi, all corneas of mice in the treatment group were lesion-free, whereas some lesions had already developed in corneas of mice in the control group. By day 11 pi, the average score of corneas of mice in the control group had increased significantly, and had reached a plateau. The average score of corneas of mice in the treatment group, in contrast increased only slightly by day 11, reaching a plateau only on day 18 pi (7 days after treatment was terminated). Moreover, the average score of corneas of mice in the treatment group actually decreased between days 28 and 36 pi, whereas the average score of corneas of mice in the control group increased during the same period. Thus, the difference in the average corneal score between Rosco-treated and control groups was of more than 2 score points at the end of the experiment (FIG. 22).

Post-Mortem Analysis of the Corneas of the Mice Treated with Rosco Revealed a Significant Degree of Protection

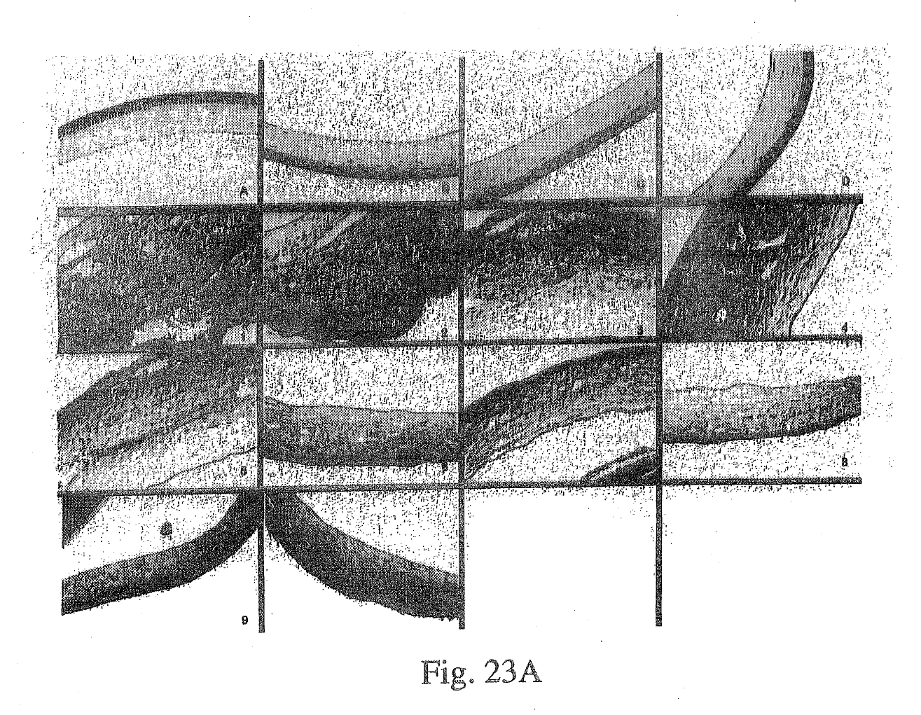

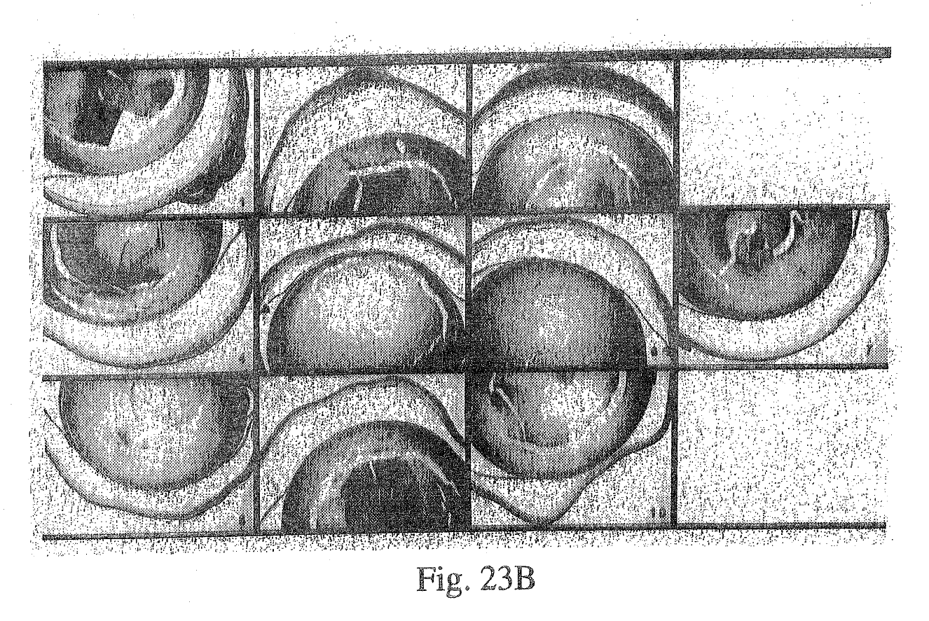

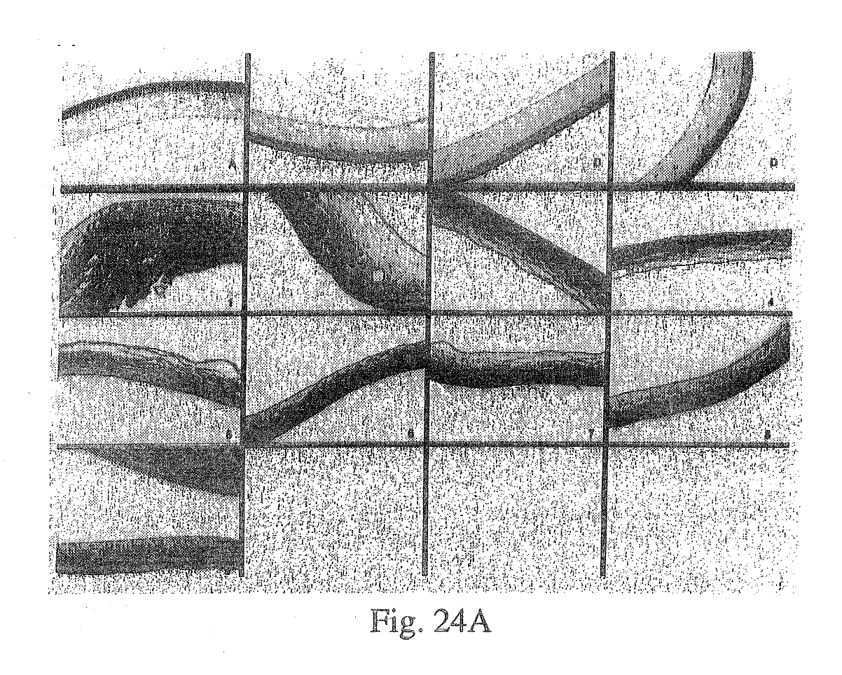

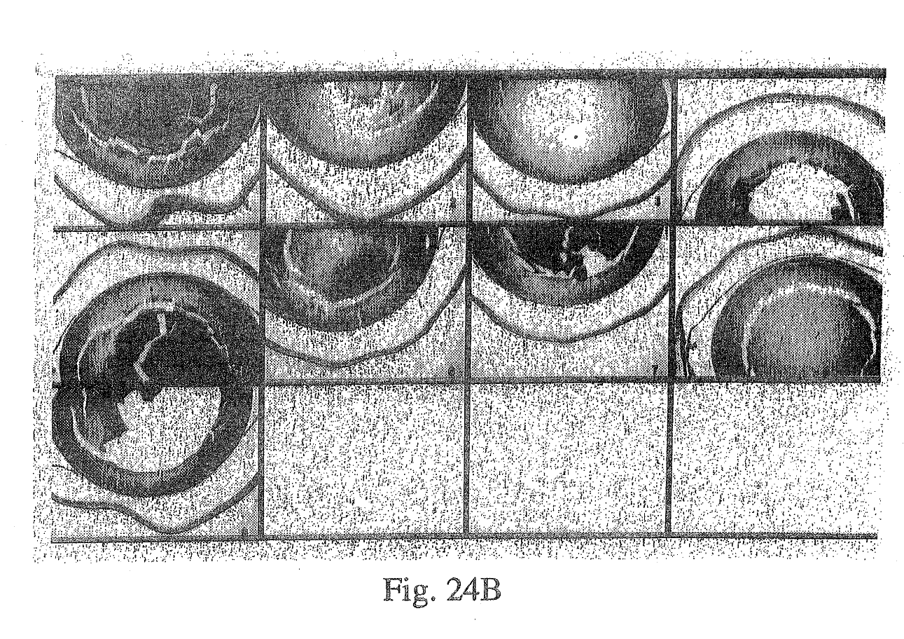

To determine unequivocally the degree of protection against HSK conferred by Rosco treatment, all infected animals were euthanized at 36 days pi, and their eyes were immediately extracted and fixed in buffered formalin at 4° C. for approximately 16 hours. Fixed eyes were dehydrated, embedded in paraffin blocks and sectioned following routine histological procedures. After staining the sectioned corneas with hematoxiline and eosin (H/E), a standard pathological microscopic evaluation was performed. For comparison, the eyes of non infected A/J mice (which are also susceptible to HSK) and the eyes of HSV infected ICR mice euthanized at 35 days pi, were included in the pathological evaluation (corneas A through D, FIGS. 23A and 24A). ICR mice do not develop HSK when infected with HSV-1, strain KOS.

As shown in FIGS. 23 and 24, the degree of corneal opacity observed during the clinical evaluations correlated well with the severity of the corneal lesions observed during post-mortem pathological examination. Thus, five corneas of mice in the control group exhibited severe thickening, total loss of the normal fibrilar structure, hypercellularity, neovascularization, massive inflammatory infiltrate, significant thickening of the corneal epithelium, destruction of Bowman's membrane and severe superficial ulcers (FIG. 23A, corneas 1 to 5). Three corneas of mice in the control group exhibited more moderate pathology, including edema, moderate inflammatory infiltrate, thickening of the corneal epithelium and loss of integrity of Bowman's membrane (FIG. 23A, corneas 6 to 8). The two remaining corneas of control mice exhibited only minor lesions, largely limited to edema and mild inflammatory infiltration (FIG. 23A, corneas 9 and 10). Moreover, in the control group, corneal lesions covered most, if not all, of the corneal surface (FIG. 23B).

In striking contrast to the mice in the control group, 6 of 9 of the corneas of mice in the treatment group showed only minor lesions under microscopic post-mortem evaluation (FIGS. 24A and 24B). Thus, in the majority of corneas of mice in this group that exhibited lesions, the lesions were limited to edema, mild hypercellularity and minor inflammatory infiltrate. Notably, the fibrilar structure of the stroma was not lost in these corneas (FIG. 24A, compare corneas A through D with corneas 3 through 9). Only two corneas from the mice in the treatment group had severe lesions (FIG. 24A, corneas 1 and 2). In one of these, moderate inflammatory infiltrate, hypercellularity and edema were observed in the stroma immediately below the epithelium, but the lesions did not extend across the corneal stroma (FIG. 24A, cornea 2). Moreover, these lesions were also restricted in area, as clearly seen in FIG. 24B (cornea 2). Only one cornea from mice in the treatment group exhibited signs of severe HSK. In this cornea, massive inflammatory infiltrate, hypercellularity, edema, neovascularization, thickening of the epithelium and destruction of Bowman's membrane were evident (FIG. 24A, cornea 1). Furthermore, the lesion in this cornea penetrated deep into the stroma, although it was clearly confined and exhibited limited lateral spread (FIG. 24B, cornea 1).

Mice Treated with Rosco During Acute HSV-1 Infection Exhibited Only Marginal and Transient Losses of Body Weight

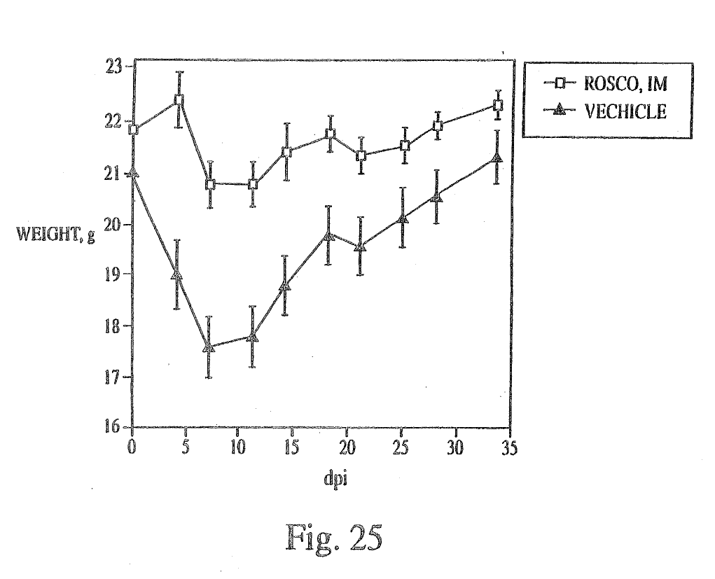

To further determine whether Rosco treatment had a positive effect on mice during the clinical course of HSV-1 infection, infected animals were weighed throughout the course of the experiment. Generally speaking, sick animals do not gain weight, but may lose it, due primarily to the combined effects of fever, and disrupted feeding habits. Indeed, in FIG. 25 the data establish that control animals lost on average, about 16% of their body weight during the acute phase of infection (days 1 to about 7 pi). Only after day 11 post-infection, did control animals begin to recover the lost weight, and only by day 28 post-infection did control animals recover their initial average body weight. In contrast, Rosco-treated animals experienced only minor loss of body weight, loosing, on average, only 4.6% of their original body weight at the peak of weight loss (days 7 to 11). Moreover, the average weight of Rosco-treated mice returned to their original weight on day 14 pi. Thus, Rosco-treated animals were spared the weight loss that frequently accompanies the illness that results from HSV infection. Moreover, these results demonstrate that Rosco at the doses used in these experiments did not have any obvious deleterious effect on the treated animals.

### EXAMPLE 6

### Roscovitine Inhibited Explant-Induced HSV Reactivation

The data presented herein establish that HSV replication requires cellular cdks that are sensitive to inhibition by the purine derivatives Olo and Rosco. Although most Rosco-sensitive cdks were believed not to be expressed in resting neurons, the question of whether Rosco-sensitive cdks are expressed in “activated” neurons which support HSV replication was examined. Expression of cdks in neurons in explanted mock or latently infected trigeminal ganglia (TG) was studied. Five to eight week old ICR mice were infected with 5×10⁵ PFU of HSV-1, strain KOS, following corneal scarification, and animals were euthanized 35 or more days pi. In the mouse ocular model, acute viral replication occurs during the one to two weeks period after infection. No actively replicating virus is normally detected in eyes or TG after day 15 pi. Thus, on day 35 post-infection, all mice are latently infected. Mice were euthanized by CO₂ inhalation and TG were immediately removed and placed in tissue culture dishes in the presence of standard tissue culture medium supplemented with antibiotics and antifungals. To study expression of specific cellular proteins in non-explanted TG neurons, some TG were fixed immediately after euthanasia. Fixation was performed overnight by rocking TG in buffered formalin at 4° C. All TG were processed (either fixed or explanted) in less than 10 minute from the time of euthanasia. At 1, 2, or 3 days pi, two explanted TG were fixed in buffered formalin as previously described. For comparison, TG from non-infected mice were processed in parallel to the TG from latently infected animals.

Fixed TG were dehydrated, embedded in paraffin blocks, sectioned and processed for immunohistology following routine histological procedures. Antibodies specific for individual cellular proteins were purchased from Santa Cruz Biotechnologies. The specificity of all antibodies was confirmed in skin samples. Furthermore, the specificity of some antibodies, including those raised against cdk-1, cdk-2, cyclin A, and cyclin B1, was also confirmed by immunoprecipitation and western blot analysis.

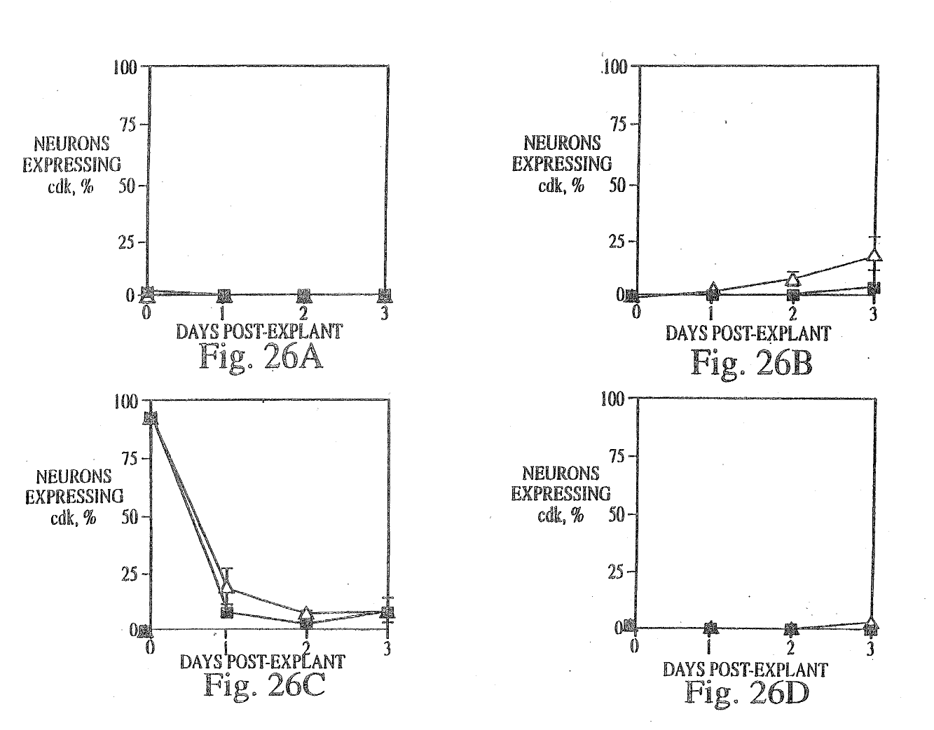

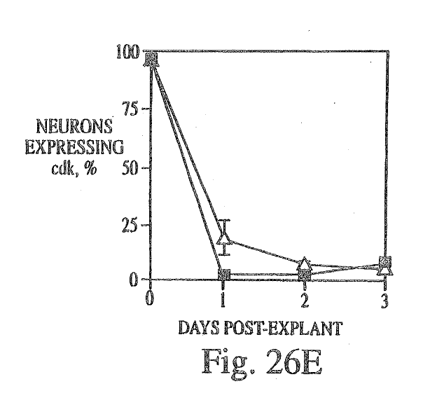

Among the Rosco-sensitive cdks, cdk-1, which had previously been reported by others be undetectable in CNS neurons, was never detected in TG neurons, regardless of whether TG were explanted or not, or for how long the period post-explant (FIG. 26, cdk-1). Similarly, cdk-5, which others had reported to be expressed in axons but not in neuronal bodies, was not detected in the bodies of TG neurons (FIG. 26, cdk-5). Cdk-3 and cdk-7 which have been discovered in the present invention to be inhibited by Rosco, were detected in a large percentage of neurons in non-explanted TG, but the percentage of neurons expressing detectable levels of these proteins decreased after explant (FIG. 26). Notably, the small number of neurons in which cdk-3 was detected on day 3 post-explant appeared to express much higher levels of this protein than that expressed by any of the many cdk-3 positive neuron prior to explant. Moreover, cdk-3 appeared to have been translocated from the cytosol to the nucleus during explant. In contrast to cdk-3 and cdk-7, cdk-2 was not detected in TG neurons before explant, consistent with what had been previously described by others in CNS neurons, but expression of cdk-2 was induced during explant. Thus, on day 3 post-explant, cdk-2 was detected in approximately 25% of neurons from latently infected, but reactivating TG (FIG. 26, cdk-2). Interestingly, in neurons, HSV antigen was detected only in cdk-2 expressing cells.

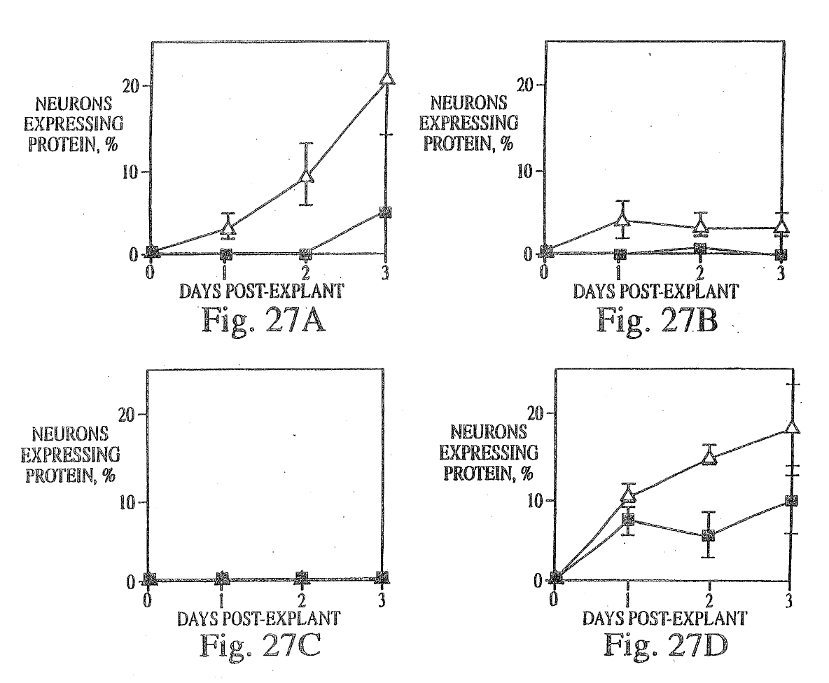

Because cdk-2 forms complexes with Cyclins A, B1, and E, it was of interest to determine whether these cyclins were also expressed in TG neurons during explant. Although none of the three cyclins was detected in neurons before explant, as reported by others for CNS neurons, Cyclins A and E, were found to be induced during explant in that these cyclins were detected in neurons from explanted TG. Thus, on day 3 post-explant, about 5 to about 20% of neurons from latently infected, but reactivating, TG exhibited detectable levels of Cyclins A and E, respectively (FIG. 27). In contrast, expression of Cyclin B 1 was not detected in neurons of explanted or non-explanted TG. Thus, this cyclin was not detected in TG neurons at any time post explant during the 3 day test period (FIG. 27).

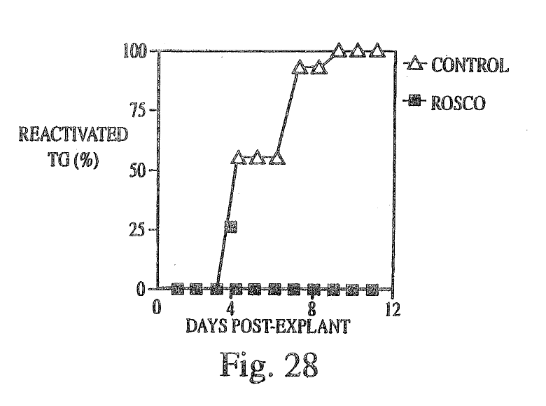

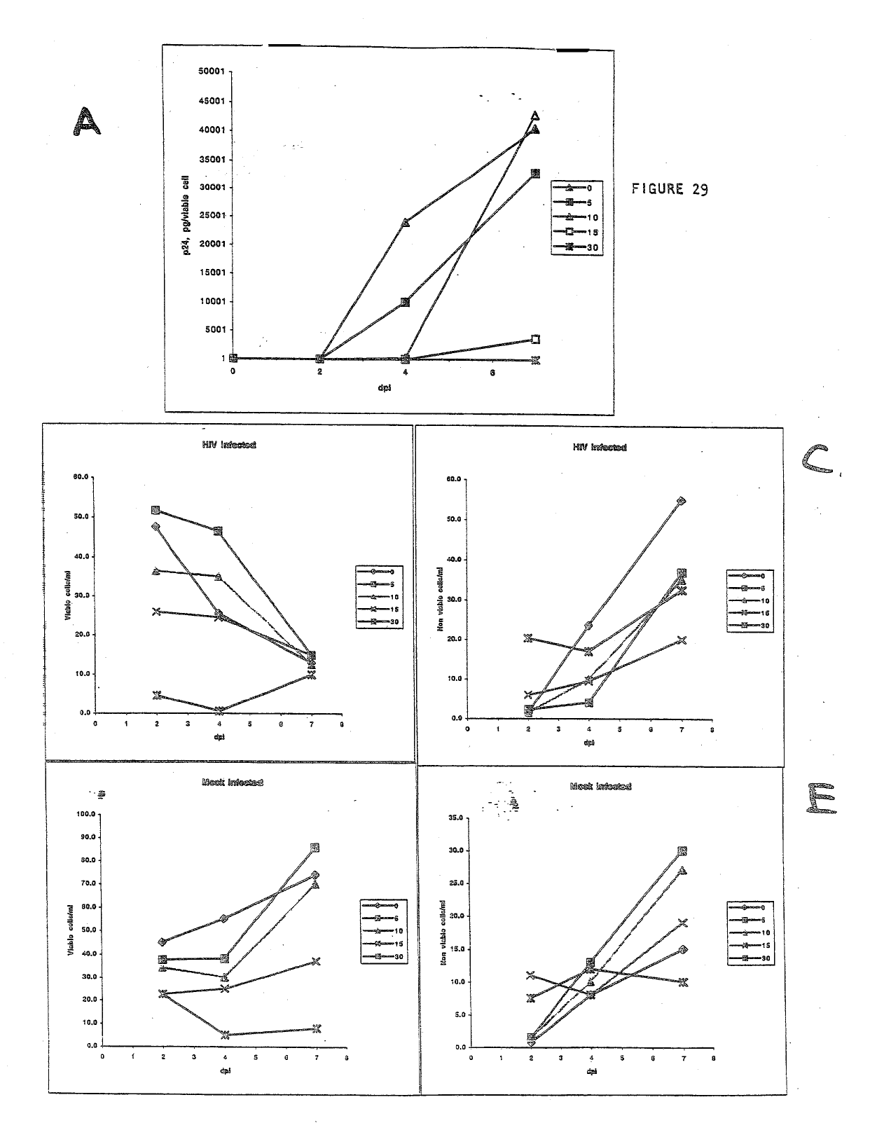

Based on the observation that several Rosco-sensitive cdks were expressed in neurons which support HSV replication during reactivation, it was of interest to determine whether Rosco inhibits reactivation of latent HSV. To test this possibility, mice were infected and euthanized after day 35 pi as described above and their TG were explanted. One half of the TG were explanted into control, drug-free, medium, whereas the other half were explanted into the same medium supplemented with 40 μM Rosco. The production of infectious virus in explanted TG cultures was monitored daily infecting fresh monolayers of susceptible Vero cells. Seven hours later, the potentially infectious inocula was removed from Vero cells and replaced with an equal volume of fresh drug-free medium. The presence of infectious virus in reactivating TG was then monitored daily by evaluating the development of HSV-specific cytopathic effect (CPE). As expected, TG explanted into standard, drug free (control) medium supported reactivation of latent HSV efficiently. Thus, more than half of the TG cultures yielded (reactivated) infectious virus by day 4 post-explant, and all explanted TG had yielded infectious virus by day 9 post-explant (FIG. 28). In contrast, HSV reactivated from none of the TG explanted in the presence of Rosco until the end of the experiment at 11 days post-explant.

In summary, Rosco-sensitive cdks were expressed in neurons under conditions in which these cells support viral replication (i.e., in explanted TG neurons), and a cdk inhibitor, Rosco, efficiently inhibited reactivation of latent HSV.

### EXAMPLE 7

### Inhibition of HIV Replication Using cdk Inhibitors

CEMX174 cells (a hybrid B/T cell line that supports replication of a spectrum of fresh clinical isolates of HIV) were infected with HIV (strain NL 4-3). One hour after infection, cultures were changed to medium containing the indicated concentrations of Rosco (μM). HIV replication was monitored by measuring p24 levels and viable and nonviable cells were counted on days 2 and 4 pi. On day 4, Rosco-containing medium was removed and replaced with drug-free medium. On day 7, p24 levels and cell counts were repeated.

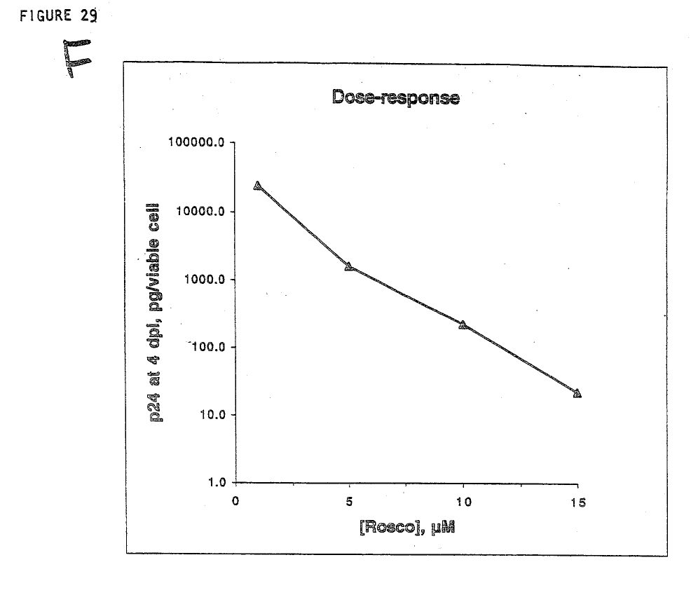

In FIG. 29A, all concentrations of Rosco tested (5, 10, 15 and 30 μM) inhibited HIV replication on day 4 as shown by the reduction in p24, pg/viable cell (˜1000 pg p24/cell for 5 μM Rosco) and undetectable for 10, 15 and 30 μM relative to the no drug control (˜2500 pg p24/cell; note that the Y axis is linear and not logarithmic).

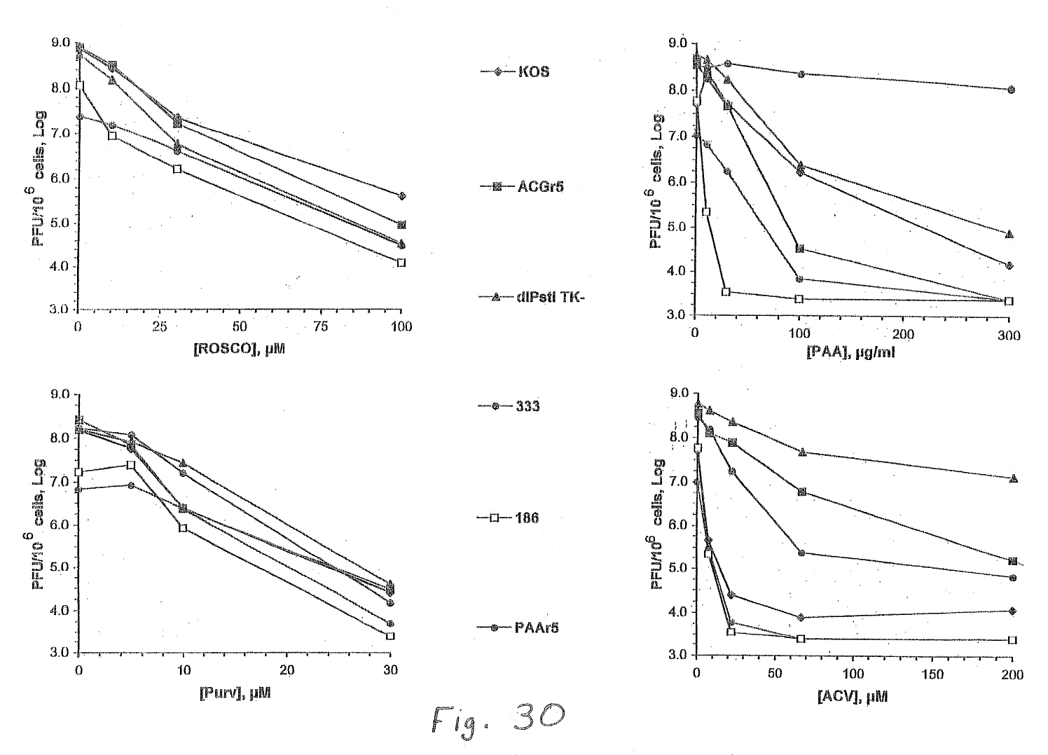

When drug was removed on day 4, p24 levels in cultures previously treated with 5 and 10 μM Rosco returned to the level of the no drug control, whereas p24 levels remained low in cultures treated with 15 and 30 μM Rosco. These findings show that as little as 5 μM Rosco inhibits HIV replication significantly, and that sufficient numbers of infected cells remained viable after drug removal to support resumption of HIV replication. The viability of HIV-infected cells dropped significantly by day 4 in cultures treated with 5 and 30 μM Rosco as well as in drug-free cultures, and continued to fall through day 7 after the change to drug-free medium (FIG. 29B). These findings demonstrate the lytic capability of HIV for CEMX174 cells, with or without Rosco. The numbers of nonviable cells in these cultures rose (FIG. 29C) in proportion to cell lysis in HIV infected cultures (FIG. 29B). As shown in FIGS. 29D and 29E, 30 μM Rosco, but not 5, 10 or 15 μM Rosco proved to be toxic for mock-infected cells.

A dose response curve of p24, pg/viable cell (on a log scale) is shown in FIG. 29F. This figure shows that 5 μM Rosco inhibited HIV replication more than 10-fold, 10 μM Rosco inhibited replication by 100-fold and 15 μM Rosco inhibited HIV replication by nearly 1000-fold.

The results of these experiments demonstrate that 5, 10, 15 and 30 μM Rosco inhibit HIV replication significantly and that upon removal of 5, 10 and 15 μM drug, cell viability was sufficient to support further HIV replication; 30 μM Rosco inhibited HIV replication but was more toxic to both HIV-infected and mock-infected cells.

### EXAMPLE 8

### Specificity of the Repression of HSV Replication by cdk Inhibitors

Inhibitors of the activities of specific cellular cdks block replication of HSV-1 at multiple points. Replication of two HSV-2 strains is also inhibited by Rosco. To test if Rosco-treated cells were unable to support replication of any virus, parallel infections were performed with HSV-1, and two viruses that encode their own transcription and DNA/RNA replication proteins, vaccinia virus and lymphocoriomeningitis virus (LCMV). Rosco efficiently inhibited replication of HSV-1, but did not inhibit replication of the remaining two viruses. Although the cellular proteins inhibited by Rosco are not required by vaccinia or LCMV, these results could also be explained if Rosco targets HSV proteins. Thus, HSV-1 mutants in the DNA polymerase gene (Pol) and in the thymidine kinase (tk) gene were tested. These proteins are the target of most anti-herpesvirus drugs. Rosco inhibited replication of acycloguanosine-resistant and TK-strains of HSV-1 efficiently. In affinity binding assays, the same spectrum of proteins bound to Purvalanol (another purine-derived cdk inhibitor closely related to Rosco, which also inhibits HSV replication) in mock and HSV-infected cells. These proteins include cdk-1, -2, -7, erks-1, -2, and cycA. Since cdk-7 is involved in cellular transcription and Rosco represses HSV transcription, the sensitivity of cdk-7 to purine-derived cdk inhibitors was examined. Rosco inhibited phosphorylation of RNA PolII by cdk-7, but not phosphorylation by cdk-8. Thus, purine-derived cdk inhibitors block replication of HSV-1 and HSV-2 specifically, but do not target viral thymidine kinase or DNA polymerase or bind to HSV proteins.

### EXAMPLE 9

### Cellular Proteins as Targets for the Treatment of Pathogens Resistant to Drugs Which Target Pathogen-Encoded Proteins.

Six groups of Vero cells were infected with 3 plaque forming units (PFU) of one of six different drug-sensitive and drug-resistant strains of HSV-1 and HSV-2. The three drug-resistant strains included ACGr5 (acyclovir-resistant), dIPst1 TK-(acyclovir-resistant) and PAAr5 (phosphonoacetic acid-resistant). The three drug-sensitive strains used were KOS (HSV-1), 186 (HSV-2) and 333 (HSV-2). After one hour of adsorption, inocula were removed, cells were extensively washed with PBS and refed with fresh medium. The medium was supplemented or not with different concentrations of either of two cellular cdk inhibitors, Roscovitine (Rosco) or Purvalanol (Purv), or with either of two antiviral drugs, Acycloguanosine (“acyclovir”—ACV) or phosphonoacetic acid (PAA). The cellular cdk inhibitor Rosco was used at concentrations up to 100 μM (FIG. 30A, upper panel) and the cellular cdk inhibitor Purv was used at concentrations up to 30 μM (FIG. 30A, lower panel). The antiviral drug PAA was used at concentrations up to 300 μg/ml (FIG. 30B, upper panel) and the antiviral drug ACV was used at concentrations up to 200 μM (FIG. 30B, lower panel). Infected cells were harvested at 24 hours post-infection and viral replication during these 24 hours was monitored by standard plaque assays.

As shown in FIG. 30A, both Rosco and Purv inhibited with the same potency replication of drug-resistant and drug-sensitive HSV. To confirm that the strains tested were indeed still resistant to ACV or PAA, additional groups of infected Vero cells were treated with different concentrations of ACV or PAA. As shown in FIG. 30B, ACV and PAA inhibited replication of antiviral drug-sensitive strains HSV efficiently, but did not inhibit replication of drug-resistant strains of HSV efficiently.

In summary, a pathogen which can be inhibited by a drug targeting a cellular cdk protein, will retain its susceptibly to a cdk inhibitor, even when the pathogen becomes resistant to a drug which targets pathogen-encoded proteins.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of inhibiting the replication of a drug-resistant pathogenic agent, said method comprising contacting a cell comprising said drug-resistant pathogenic agent with a compound capable of inhibiting replication of said drug-resistant pathogenic agent, wherein said compound targets a cellular protein, thereby inhibiting replication of said drug-resistant pathogenic agent.

2. The method of claim 1, wherein said drug-resistant pathogenic agent is selected from the group consisting of a virus, a bacterium, a fungus, a yeast and a parasite.

3. The method of claim 2, wherein said drug-resistant pathogenic agent is a virus.

4. The method of claim 3, wherein said virus is selected from the group consisting of a herpesvirus, a hepatitis B virus, a hepatitis C virus, a human papilloma virus, human immunodeficiency virus (HIV) and human T-cell leukemia virus (HTLV).

5. The method of claim 4, wherein said virus is HIV.

6. The method of claim 4, wherein said virus is a herpesvirus.

7. The method of claim 6, wherein said herpesvirus is selected from the group consisting of herpes simplex virus type 1 (HSV-1), herpes simplex virus type 2 (HSV-2), cytomegalovirus, varicella zoster virus (VZV), bovine herpesvirus type 1 (BHV-1), equine herpesvirus type 1 (EHV-1), pseudorabiesvirus (PRV), Epstein Barr virus, human herpesvirus type 6, human herpesvirus type 7 and human herpesvirus type 8.

8. The method of claim 7, wherein said herpesvirus is HSV.

9. The method of claim 8, wherein said herpesvirus is HSV-1.

10. The method of claim 1, wherein said compound is a cdk inhibitor.

11. The method of claim 10, wherein said cdk inhibitor is selected from the group consisting of 6-dimethylaminopurine, isopentenyladeninne, olomoucine, roscovitine, CVT-313, purvalanol A&B, flavopiridol, suramin, 9-hydroxyellipticine, toyocamycin, staurosporine, y-butyrolactone, CGP60474, kenpaullone, alsterpaullone, indirubin-3′-monoxime and hymenialdisine.

12. The method of claim 10, wherein said cdk inhibitor is selected from the group consisting of roscovitine, olomoucine, and provalanol.

13. The method of claim 1, wherein said compound is a non-cdk inhibitor.

14. A method of inhibiting the replication of a drug-resistant pathogenic agent in a mammal, said method comprising administering to said mammal a therapeutically effective amount of a compound capable of inhibiting replication of said drug-resistant pathogenic agent, wherein said compound targets a cellular protein, thereby inhibiting replication of said drug-resistant pathogenic agent in said mammal.

15. The method of claim 14, wherein said mammal is a human

16. The method of claim 14, wherein said method of administering said compound is via a route selected from the group consisting of oral, rectal, vaginal, parenteral, topical, pulmonary, intranasal, buccal, ophthalmic and intrathecal.
